(19) United States
(12) Patent Application Publication
(10) Pub. No.: US 2022/0185974 A1
(43) Pub. Date: Jun. 16, 2022

(54) WAVELENGTH CONVERSION FILM, WAVELENGTH CONVERSION DEVICE, LIGHT-EMITTING MEMBER, AUTHENTICATION DEVICE, WRISTBAND-TYPE ELECTRONIC DEVICE AND BIOMETRIC DEVICE

(71) Applicant: Konica Minolta, Inc., Tokyo (JP)

(72) Inventors: Ryo NAKABAYASHI, Hachioji-shi, Tokyo (JP); Yasuo MIYATA, Yokohama-shi, Kanagawa (JP); Kenichi TABATA, Inagi-shi, Tokyo (JP)

(21) Appl. No.: 17/440,071

(22) PCT Filed: Mar. 17, 2020
(86) PCT No.: PCT/JP2020/011704

§ 371 Date: Sep. 16, 2021

(30) Foreign Application Priority Data
Mar. 19, 2019 (JP) 2019-051500

(51) Int. Cl. C08J 5/18; C09K 11/06; H01L 51/00; A61B 5/1171; A61B 5/00; A61B 5/1455; H01L 51/50

(52) U.S. Cl. CPC C08J 5/18; G06V 40/14; H01L 51/0035; H01L 51/006; H01L 51/0053; H01L 51/0059; H01L 51/0054; A61B 5/1171; A61B 5/681; A61B 5/14552; H01L 51/5036; H01L 51/0058; C09K 2211/1007; C09K 2211/1011; C09K 2211/1014; C09K 2211/1018; C08J 2325/06; C09K 11/06

(57) ABSTRACT

Provided is a wavelength conversion film containing a luminescent dye, wherein, in an absorption spectrum and an emission spectrum, when the spectrums are normalized so that two maximum intensities of an absorption band having a maximum absorption wavelength of a maximum intensity and an emission band having a maximum emission wavelength of a maximum intensity become the same intensity, and when a molar extinction coefficient at an intersection where the normalized absorption band and the normalized emission band overlap in the spectrum is ε, and a content of the luminescent dye included according to a film thickness per 1 cm² of the wavelength conversion film is C (mol), the wavelength conversion film satisfies the following Expression (1),

1.0×10⁻³≤ε×C. Expression (1)

### TECHNICAL FIELD

The present invention relates to a wavelength conversion film, a wavelength conversion device, a light-emitting member, an authentication device, a wristband-type electronic device, and a biometric device. More particularly, the present invention relates to a wavelength conversion film which controls an emission waveform and has a good quantum yield.

### BACKGROUND

At present, a wavelength conversion film for converting the wavelength of light is known. This type of wavelength conversion film has a function of converting the wavelength of light by absorbing light of a predetermined range of wavelength and emitting light of another range of wavelength.

In the prior art, the emission waveform of the wavelength conversion film is defined only by the luminescent dye contained in the film. As a result, in the wavelength conversion film, when the emission waveform is changed, it is necessary to change the luminescent dye every time, and it is difficult to easily control the maximum emission wavelength within a range of several tens of nm and to narrow the half-value width of the emission waveform to improve the color purity.

Therefore, in order to change the luminescent dye, it is necessary to perform a validation cycle of molecular design, synthesis, and evaluation of the luminescent dye, and as a result, a huge development period has been required. Furthermore, it has been sometimes difficult to emit light with a desired emission color due to molecular design. In addition, the conventional technique has the above-mentioned problem in terms of control of the emission waveform.

Regarding the modification of the emission waveform, in the prior art, excimer light emission and exciplex light emission due to the interaction between fluorescent materials are known, but these luminescence have a long luminescence lifetime of 100 ns or more. Since the time of being in an unstable excited state is at least 10 times longer than that of general fluorescence emission, there is a problem in the durability of the material. Further, it is generally known that excimer light emission and ti exciplex light emission are accompanied by a large structural change in an excited state, so that excitons are easily deactivated without radiation and the quantum yield is low.

A luminescence converter is disclosed as a wavelength conversion film for a fluorescent material-enhanced light source having a high color rendering index. However, in the wavelength conversion film described in Patent Document 1, since a self-absorption phenomenon loses luminescence, an invention for compensating for a loss thereof is disclosed. Also in the patent relating to the wavelength conversion of Patent Document 2, a phenomenon is described in which the emission of the dye is changed by the self-absorption phenomenon, but it is noted that it is preferable to limit the self-absorption which increases the loss due to the nonradiative relaxation.

In the prior art, the self-absorption phenomenon is a phenomenon in which loss compensation is necessary or avoided, and an invention of a light-emitting film in which an emission waveform is controlled by actively utilizing the self-absorption phenomenon is not disclosed. Therefore, there has been no specific proposal for designing by controlling specific parameters for effectively expressing the self-absorption phenomenon, for example, the dye amount, the dye concentration, and the molar extinction coefficient, and a specific example thereof has not been disclosed in the prior art document (for example, refer to Patent Document 2).

### PRIOR ART DOCUMENTS

### Patent Documents

Patent Document 1: Japanese Patent No. 6032766
Patent Document 2: Japanese Patent No. 6273287

### SUMMARY OF THE INVENTION

### Problems to be Solved by the Invention

The present invention has been made in view of the above problems and status, an object of the present invention is to provide a wavelength conversion film enabling compatibility of good quantum yield and emission waveform control, a wavelength conversion device having the same, a light-emitting member, an authentication device, a wristband-type electronic device and a biometric device having the same wavelength conversion film.

### Means to Solve the Problems

As a result of examining the cause of the above-mentioned problem, the present inventor has conceived an invention capable of newly controlling the self-absorption phenomenon and found that the above-mentioned problem was solved. That is, in the absorption and emission spectrums, the spectrums are normalized so that the two maximum intensities of the absorption band having the maximum absorption wavelength of the maximum intensity and the emission band having the maximum emission wavelength of the maximum intensity become the same intensity, and when the molar extinction coefficient at the intersection where the normalized absorption band and the emission band overlap in the spectrum is ε, and the content of the emission dye included according to a film thickness per 1 cm² of the film area is C (mol), it was found that a wavelength conversion film having a value of ε×C within a specific range can control the emission waveform and realize a wavelength conversion film having a good quantum yield, and the present invention has been achieved.

In other words, the above problem according to the present invention is solved by the following means.

1. A wavelength conversion film containing a luminescent dye, wherein, in an absorption spectrum and an emission spectrum, when the spectrums are normalized so that two maximum intensities of an absorption band having a maximum absorption wavelength of a maximum intensity and an emission band having a maximum emission wavelength of a maximum intensity become the same intensity, and when a molar extinction coefficient at an intersection where the normalized absorption band and the normalized emission band overlap in the spectrum is ε, and a content of the luminescent dye included according to a film thickness per 1 cm² of the film area is C (mol), the wavelength conversion film satisfies the following Expression (1),

1.0×10⁻³≤ε×C. Expression (1)

2. The wavelength conversion film described in item 1, wherein ε×C is 1.5×10⁻³ or more.
3. The wavelength conversion film described in item 1 or 2, wherein wen a reflection type emission spectrum detected by a configuration in which an excitation light source and a detection unit are arranged on the same surface side with respect to the wavelength conversion film is defined as an emission spectrum A, and a transmission type emission spectrum detected by a configuration in which the excitation light source and the detection unit are arranged at positions facing each other across the wavelength conversion film is defined as an emission spectrum B, and when peak values of the maximum emission wavelengths of the respective emission spectrums are aligned, a wavelength difference value Δλ at an intensity of 20% on a short-wave side of each of the emission spectrum A and the emission spectrum B is 10 nm or mom.
4. The wavelength conversion film described in item 1 or 2, wherein the wavelength difference value Δλ at an intensity of 20% of the short-wave side of the spectrums is 15 nm or more.
5. The wavelength conversion film described in any one of items 1 to 4, wherein an absolute quantum yield is 10% or more.
6. The wavelength conversion film described in any one of items 1 to 4, wherein an absolute quantum yield is 20% or more.
7. The wavelength conversion film described in any one of items 1 to 6, wherein the maximum emission wavelength is 750 nm or mom.
8. The wavelength conversion film described in any one of items 1 to 6, wherein the maximum emission wavelength is 800 nm or more.
9. The wavelength conversion film described in any one of items 1 to 8, wherein the wavelength conversion film is formed of the luminescent dye and a transparent matrix resin.
10. The wavelength conversion film described in item 9, wherein the transparent matrix resin is at least one resin selected from a thermosetting resin, a thermoplastic resin, or a photocurable resin.
11. A wavelength conversion device comprising the wavelength conversion film described in any one of items 1 to 10.
12. A light-emitting member comprising the wavelength conversion film described in any one of items 1 to 10.
13. The light-emitting member described in item 12, wherein the light source in the light-emitting member is an organic electroluminescent element.
14. An authentication device comprising the light-emitting member described in item 12 or 13.
15. A wristband-type electronic device comprising the authentication device described in item 14, wherein biometric authentication is performed by imaging a wrist vein.
16. The wristband-type electronic device described in item 15, wherein the light source is provided on any place of the wristband other than on the same plane as an imaging unit when mounting the device.
17. The biometric device comprising the light-emitting member described in item 12 or 13.
18. The biometric device described in item 17 being a pulse oximeter that performs measurement at a base of a wrist or a finger.
19. The biometric device described in item 17 being a pulse wave sensor that performs measurement at the base of a wrist or a finger.

### Effects of the Invention

According to the above-mentioned means of the present invention, it is possible to provide a wavelength conversion film having a good quantum yield by controlling an emission waveform, and a wavelength conversion device, a light-emitting member, an authentication device, a wristband-type electronic device, and a biometric measurement device provided with the wavelength conversion film.

With respect to the expression mechanism and the action mechanism of the effect which may solve the above problem by setting the production condition defined in the present invention, the following speculation is made.

The wavelength conversion film of the present invention contains a luminescent dye, and in an absorption spectrum and an emission spectrum, the spectrums are normalized so that the absorption band having the maximum absorption wavelength of the maximum intensity and the emission band having the maximum emission wavelength of the maximum intensity have the same intensity. When the molar extinction coefficient at the intersection point where the normalized absorption band and the emission band overlap in the spectrum is ε, and the content of the emission dye according to a film thickness per 1 cm² of the film area is C (mol), the above Expression (1) is satisfied.

More specifically, in the present invention, in the formation of the wavelength conversion film, one or more kinds of luminescent dyes are used, and the molar extinction coefficient, the overlap region of absorption and emission, the film thickness, and the luminescent dye concentration are appropriately controlled with respect to a specific luminescent dye, whereby it was achieved high color purification by amplifying the long wavelength side peak of the emission waveform from a specific luminescent dye and narrowing the emission spectrum for fluorescence emission with a good quantum yield, and it is possible to control the luminescent waveform which is difficult in the wavelength conversion film of the prior art and to provide a wavelength conversion film having a good quantum yield.

In order to develop the self-absorption phenomena in the wavelength conversion film, it is required to repeat many cycles of reabsorption→remission→reabsorption of light in the wavelength range in which the emission spectrum and the absorption spectrum overlap among the light-emitting components of the luminescent dye until the light entering the wavelength conversion film passes through the film.

Therefore, in order to efficiently proceed reabsorption, it is advantageous that the absorption ability of light in the wavelength conversion film is higher, and it is preferable that the molar extinction coefficient of the luminescent dye itself is higher. However, when the emission spectrum and the absorption spectrum overlap at a position where the intensity is low (overlap is small), the molar extinction coefficient of the absorbed region is low, and therefore the molar extinction coefficient at the spectral intersection point rather than the peak top becomes important.

In addition, as a method of increasing the absorption ability, the amount of luminescent dye contained in the wavelength conversion film is also an important factor. The larger the amount of the luminescent dye, the higher the probability of reabsorption of the luminescent light, which is advantageous. This amount of luminescent dye is a factor which varies depending on the concentration of luminescent dye in the wavelength conversion film and the film thickness.

From the above, in the present invention, it has been found that the self-absorption phenomenon is strongly expressed in a range in which the molar extinction coefficient and the luminescent dye amount are represented by two kinds of parameters, and a value obtained by multiplying these two kinds of parameters is equal to or more than a predetermined value.

Further, in the wavelength conversion film of the present invention, when the emission spectrum A is detected in a configuration in which the excitation light source and the detection unit are arranged on the same surface side with respect to the wavelength conversion film, and the emission spectrum B is detected in a configuration in which the excitation light source and the detection unit are arranged at positions facing each other across the wavelength conversion film, and when the peak values of the maximum emission wavelengths of the respective emission spectrums are aligned, the wavelength difference value Δλ at an intensity of 20% on a short-wave side of each of the emission spectrum A and the emission spectrum B is preferably 10 nm or more.

This is because, as will be described later in FIG. 3A. FIG. 3B and FIG. 4, by taking the difference in wavelength change between the transmissive type, which tends to cause self-absorption, and the reflective type, which does not easily occur, it is possible to determine the effect of the self-absorbing phenomenon by the difference in the measuring method of the transmission type and the reflection type using a single film. As a result, it is possible to easily control the emission waveform and design a wavelength conversion film having a good quantum yield.

### BRIEF DESCRIPTION OF THE DRAWINGS

FIG. 1 is a graph for explaining the molar extinction coefficient ε defined in the present invention.

FIG. 2 is a graph showing an example of the relationship between the film thickness of the wavelength conversion film and the emission wavelength.

FIG. 3A is a schematic diagram showing an exemplary measuring apparatus of a reflection type emission spectrum.

FIG. 3B is a schematic diagram showing an exemplary measuring apparatus of a transmission type emission spectrum.

FIG. 4 is a diagram showing an example of an emission wavelength profile by an emission spectrum A of the reflection type of the wavelength conversion film of the present invention produced in Example and an emission spectrum B of the transmission type.

FIG. 5 is a schematic cross-sectional view showing an example of a configuration of a light-emitting member having a wavelength conversion film of the present invention.

FIG. 6A is a scatter diagram showing the relation between the dye amount (C)×the molar extinction coefficients (ε) of the luminescent dye used in the Examples and the wavelength change width Δλ at 20% luminescence intensity.

FIG. 6B is an enlarged view of the relation between the dye amount (C)×the molar extinction coefficient (ε) of the luminescent dye shown in FIG. 6A and the wavelength change width Δλ at 20% luminescence intensity in the threshold area.

FIG. 7 is a diagram showing an example of an emission wavelength profile by the emission spectrum A of the reflection type of the wavelength conversion film of the comparative example prepared in Example 1, and an emission wavelength profile by the emission spectrum B of the transmission type.

FIG. 8 is a graph showing an emission spectrum of the wavelength conversion film produced in Example 2.

FIG. 9 is a schematic cross-sectional view showing an example of a configuration of a light-emitting member including a cut filter and the wavelength conversion film of the present invention.

FIG. 10 is a perspective view showing an example of the configuration of a wristband-type electronic device. FIG. 11A is a schematic diagram showing an example of transmission type sensing at a fingertip using a point light source.

FIG. 11B is a schematic diagram showing an example of transmission type sensing at a fingertip using a surface light source.

FIG. 11C is a schematic diagram showing an example of reflection type sensing at a fingertip using a surface light source.

FIG. 12A is a schematic diagram showing an example of transmission type sensing at a base of a finger using a point light source.

FIG. 12B is a schematic diagram showing an example of transmission type sensing at a base of a finger using a surface light source.

FIG. 13A is a schematic diagram showing an example of V-shaped sensing on a wrist using a point light source.

FIG. 13B is a schematic diagram showing an example of V-shaped sensing on a wrist using a surface light source. FIG. 13C is a schematic diagram showing an example of reflection type sensing on a wrist using a surface light source.

FIG. 13D is a schematic diagram showing an example of reflection type sensing on a wrist using a point light source.

### EMBODIMENTS TO CARRY OUT THE INVENTION

The wavelength conversion film of the present invention is a wavelength conversion film containing a luminescent dye, and is characterized in that, in the absorption spectrum and emission spectrum, the spectrums are normalized so that the two maximum intensities of an absorption band having a maximum absorption wavelength of maximum intensity and an emission band having a maximum emission wavelength of maximum intensity are the same intensity, when the molar extinction coefficient at the intersection point where the normalized absorption band and the emission band overlap in the spectrum is E, and the content of the luminescent dye contained according to a film thickness per 1 cm² of the film area is C (mol), ε×C is 1.0×10⁻³ or more. This feature is a technical feature common to or corresponding to each of the following embodiments.

As an embodiment of the present invention, from the viewpoint of expressing the effect of the present invention, it is also preferable that the product value (ε×C) of the molar extinction coefficient ε and the amount of luminescent dye C (mol) is 1.5×10⁻³, or more in terms of more excellent self-absorption phenomena may be exhibited.

Further, the wavelength conversion film of the present invention preferably has an absolute quantum yield of 10% or more, and more preferably 20% or more in terms of enabling the objective effect of the present invention to be further exhibited.

Further, when the reflection type emission spectrum detected by the configuration in which the excitation light source and the detection unit are disposed on the same surface side with respect to the wavelength conversion film is an emission spectrum A; the transmission type emission spectrum detected by the configuration in which the excitation light source and the detection unit are disposed at positions facing each other across the wavelength conversion film is an emission spectrum B; and when aligning the peak value of the maximum emission wavelength of the respective emission spectrums, the wavelength difference value Δλ at an intensity of 20% on the short-wave side of the emission spectrum A and the emission spectrum B is preferably 10 nm or more.

Further, it is more preferable that the wavelength difference value Δλ at an intensity 20% of the short-wave side is 15 nm or more in view of exhibiting a more excellent self-absorption phenomenon.

In addition, the wavelength conversion film of the present invention is preferable in that it may be effectively applied to a color filter such as an agricultural vinyl house capable of promoting cultivation of a crop, or a display, from the viewpoint that the emission waveform and the emission spectrum width may be transformed into a desired shape. In particular, in light-emission in the infrared region, since the emission waveform may be transformed into longer wavelength light-emission, it is preferable that the maximum emission wavelength has an emission wavelength in the near-infrared region of 750 nm or more, more preferably 800 nm or more, because the living body transparency of light may be improved, it may be effectively applied to a sensor or a light source in the near-infrared region, for example, a biometric authentication device (e.g., a vein authentication device), a biometric device (e.g., a pulse wave sensor, a pulse oximeter), or a phototherapy.

Further, it is preferable that the wavelength conversion film of the present invention is formed of a luminescent dye and a transparent matrix resin, and that the transparent matrix resin is at least one resin selected from a thermosetting resin, a thermoplastic resin, or a photocurable resin in view of obtaining a wavelength conversion film having excellent durability.

Hereinafter, detailed descriptions of the present invention, its constituent elements, configurations and embodiments for carrying out the present invention will be given. In the present application. “to” is used in the meaning that the numerical values described before and after are included as a lower limit value and an upper limit value. The dimensional ratios in the drawings are exaggerated for convenience of description, and may differ from actual ratios.

### <<Characteristics of Wavelength Conversion Film>>

The wavelength conversion film of the present invention is characterized in that it contains at least one luminescent dye, and the product value (ε×C) of the molar extinction coefficient e and the luminescent dye amount C (mol) is 1.0×10⁻³ or more, where e is the molar extinction coefficient at the intersection of the absorption band having the maximum absorption wavelength of the maximum intensity with normalized peak value of the maximum emission wavelength and the emission band having the maximum emission wavelength of the maximum intensity, and C (mol) is the luminescent dye amount contained according to a film thickness per 1 cm² of the film area.

First, each characteristic value of the wavelength conversion film defined in the present invention will be described.

[Product Value (ε×C) of Molar Extinction Coefficient ε and Luminescent Dye Content C (mol)]

The wavelength conversion film of the present invention is characterized in that, in the absorption and emission spectrums, the spectrums are normalized so that the two maximum intensities of the absorption band having the maximum absorption wavelength of the maximum intensity and the emission band having the maximum emission wavelength of the maximum intensity have the same intensity, the molar extinction coefficient at the intersection point where the normalized absorption band and the emission band overlap in the spectrum is ε, and when the content of the luminescent dye included according to a film thickness per 1 cm² of film area is C (mol), the product value (ε×C) of the molar extinction coefficient ε and the luminescent dye amount C (mol) is 1.0×10⁻³ or more.

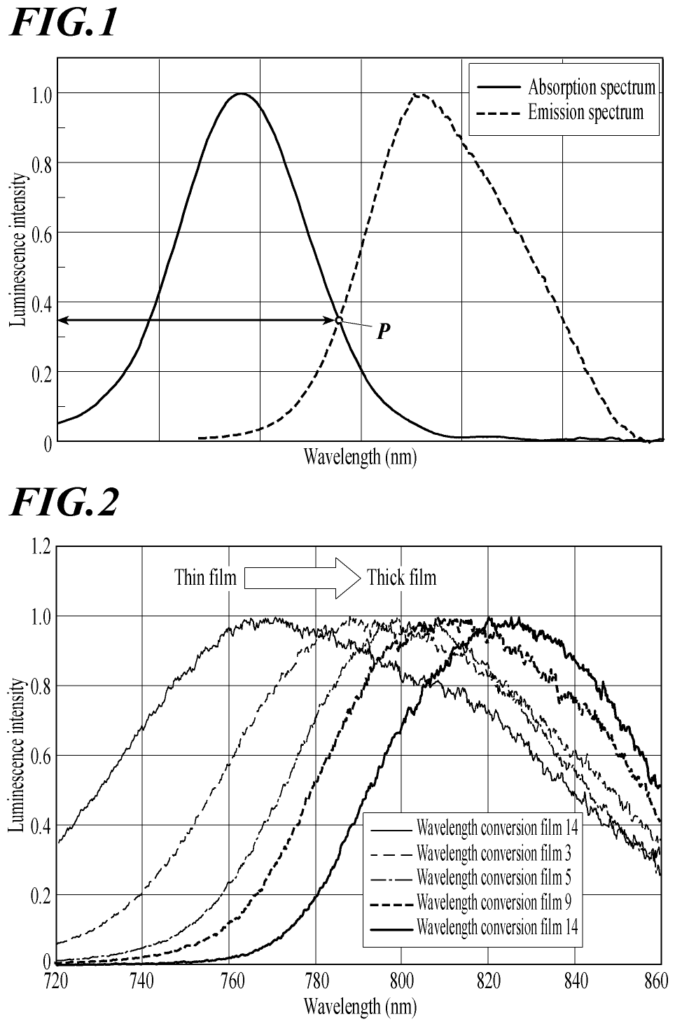

The molar extinction coefficient ε referred to in the present invention will be described with reference to FIG. 1.

FIG. 1 is an example of a graph for explaining the molar extinction coefficient e defined in the present invention.

In FIG. 1, the horizontal axis represents the wavelength (nm), the vertical axis represents the emission/absorption intensity, the waveform of the absorption spectrum of the luminescent dye is represented by a solid line, and the waveform of the emission spectrum is represented by a broken line, and the spectrums are normalized so that the absorption band having the maximum absorption wavelength of the maximum intensity and the two maximum intensities of the emission band having the maximum emission wavelength of the maximum intensity become the same intensity in the absorption and emission spectrums, and the molar extinction coefficient at the intersection point P where the normalized absorption band and the emission band overlap in the spectrum is defined as ε.

In order to realize the self-absorption phenomena with desired strengths, as described above, it is required to repeat many cycles of reabsorption→reemission→reabsorption of light in the wavelength range in which the emission spectrum and the absorption spectrum overlap among the light-emitting components of the luminescent dye until the light entering the wavelength conversion film passes through the film.

Therefore, in order to efficiently proceed reabsorption, it is advantageous to have a higher light absorption ability in the wavelength conversion film, and it is preferable to have a higher molar extinction coefficient of the luminescent dye itself. However, when the emission spectrum and the absorption spectrum overlap at a position of low intensity, since the molar extinction coefficient of the absorbed region is low, the molar extinction coefficient at the intersection point P of the spectrum rather than the peak top becomes an important factor.

Therefore, in the present invention, as one factor for determining the self-absorption phenomenon characteristics, the molar extinction coefficient E described above is used as one important factor.

In addition, as a method of controlling the emission wavelength by using a specific luminescent dye in the wavelength conversion film, the amount of luminescent dye included in the wavelength conversion film according to a film thickness per unit area also becomes an important factor. The larger the amount of the luminescent dye, the higher the probability of reabsorption of the luminescent light, which is advantageous. The amount of the luminescent dye is a factor whose wavelength fluctuates depending on the luminescent dye concentration and the film thickness in the wavelength conversion film.

FIG. 2 is a graph showing an example of the relationship between the film thickness of the wavelength conversion film and the emission wavelength, which has a characteristic that the emission peak of the luminescent dye becomes longer as the film thickness of the wavelength conversion film becomes thicker, and by applying this characteristic, the emission spectrum (emission peak) of the wavelength conversion film may be controlled to a desired condition by appropriately adjusting the dye amount even for one type of luminescent dye.

That is, in the present invention, the likelihood of self-absorption phenomenon is expressed by two parameters, the molar extinction coefficient and the amount of luminescent dye, and by setting the product value (ε×C) of these two parameters, the molar extinction coefficient ε and the amount of luminescent dye C (mol) to 1.0×10⁻³ or more, the self-absorption phenomenon, may be strongly expressed.

The molar extinction coefficient ε according to the present invention may be measured according to the following method.

As for the value of the molar extinction coefficient of the luminescent dye, an absorption spectrum is measured using a spectrophotometer (e.g., U-3300, manufactured by Hitachi High-Tech Science Corporation) for a solution obtained by adjusting the dye concentration so that the absorbance is within a range of 1.0 or less and dissolving the solution in a solvent, and a value of the molar extinction coefficient serving as a reference is measured from the dye concentration.

In order to obtain the intersection point P of the emission spectrum and the absorption spectrum, the emission spectrum and the absorption spectrum are measured from a wavelength conversion film in which the dye concentration is adjusted so that the dye amount (C)×molar extinction coefficient (ε) is in the range of 1.0×10⁻⁴ or less by using a fluorometer (for example, F-7000, manufactured by Hitachi High-Tech Science Corporation) and a spectrophotometer (for example, U-3300, manufactured by Hitachi High-Tech Science Corporation).

Next, after normalizing the peak intensities of the maximum emission wavelengths of the absorption spectrum and the emission spectrum obtained from the above wavelength conversion film to the same intensity as shown in FIG. 1, the intersection point P of the two spectrums is obtained, and the molar extinction coefficient at the same absorption intensity in the normalized absorption spectrum of the solution at the absorption intensity of the intersection P is defined as the molar extinction coefficient E of the luminescent dye.

### [Absolute Quantum Yield]

The quantum yield is a value indicating how efficiently light emission may be obtained with respect to the absorbed light (energy). The quantum yield may be calculated by measuring the number of photons absorbed by the sample and the number of photons emitted.

In the wavelength conversion film of the present invention, it is preferable that the absolute quantum yield is 10% or more.

By applying a luminescent dye having an absolute quantum yield of 10% or mom to the wavelength conversion film, high wavelength conversion efficiency may be exhibited.

In the present invention, further, the absolute quantum yield is mom preferably 20% or more, still more preferably 25% or more, and particularly preferably 30% or more.

The absolute quantum yield of the luminescent dye according to the present invention may be determined by measuring according to the following method.

A coating film containing a luminescent dye is prepared and measured using a “Quantaurus-QY absolute PL quantum yield measuring apparatus: C11347-01” (manufactured by Hamamatsu Photonics K.K), and an absolute quantum yield of a luminescent dye is measured.

### [Wavelength Difference Value Δλ Between Reflection Type Emission Spectrum a and Transmission Type Emission Spectrum B]

In the wavelength conversion film of the present invention, the reflection type emission spectrum detected by the configuration in which the excitation light source and the detection unit are disposed on the same surface side with respect to the wavelength conversion film is an emission spectrum A, the transmission emission spectrum detected by the configuration in which the excitation light source and the detection unit are disposed at positions facing each other across the wavelength conversion film is an emission spectrum B, and when aligning the peak values of the maximum emission wavelength of the respective emission spectrums, it is a preferred embodiment that the wavelength difference value Δλ at an intensity of 20% on the short-wave side of each of the emission spectrum A and the emission spectrum B is 10 n nor more.

An emission spectrum A, which is a reflection type emission spectrum, and an emission spectrum B, which is a transmission type emission spectrum, will be described.

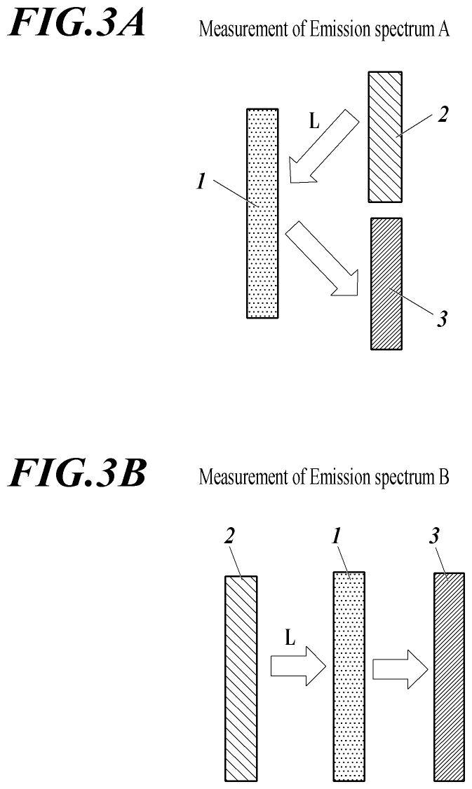

FIG. 3A shows a schematic diagram showing an exemplary measuring apparatus of a reflection type emission spectrum, and FIG. 3B shows a schematic diagram showing an exemplary measuring apparatus of a transmission type emission spectrum.

The measuring apparatus shown in FIG. 3A has an apparatus configuration used for measuring an emission spectrum A which is a reflection type emission spectrum, in which the excitation light source 2 and the detection unit 3 are arranged on the same surface side at positions facing the surface of the wavelength conversion film 1, and in which the excitation light L irradiated from the excitation light source 2 excites the dye on the surface of the wavelength conversion film 1, and the light emission is received by the detection unit 3, thereby measuring the reflection type emission spectrum A. In the emission spectrum A, since the excitation light L excites the dye on the surface of the wavelength conversion film 1, the proportion of the generated emission component that directly enters the detection unit 3 increases. Therefore, an emission spectrum in which the contribution of the self-absorption phenomenon is low may be obtained.

On the other hand, the measuring apparatus shown in FIG. 3B has an apparatus configuration used for measurement of the emission spectrum B, which is a transmission type emission spectrum, in which the excitation light source 2 and the detection unit 3 are arranged at positions facing each other with the wavelength conversion film 1 interposed therebetween, and the excitation light L is irradiated from the back surface of the wavelength conversion film 1 by the excitation light source 2, for example, LEDs, and all the light transmitted through the wavelength conversion film 1 is received by the detection unit 3, thereby measuring the transmission type emission spectrum B.

In the transmission type emission spectrum B, all of the excitation light passes through the wavelength conversion film 1, so that an emission spectrum capable of reflecting the contribution of the self-absorption phenomenon may be obtained. In the case of the wavelength conversion film which causes the self-absorption phenomenon, the emission spectrum B is obtained from the emission spectrum A as a spectrum in which the emission waveform is deformed to the long wavelength side.

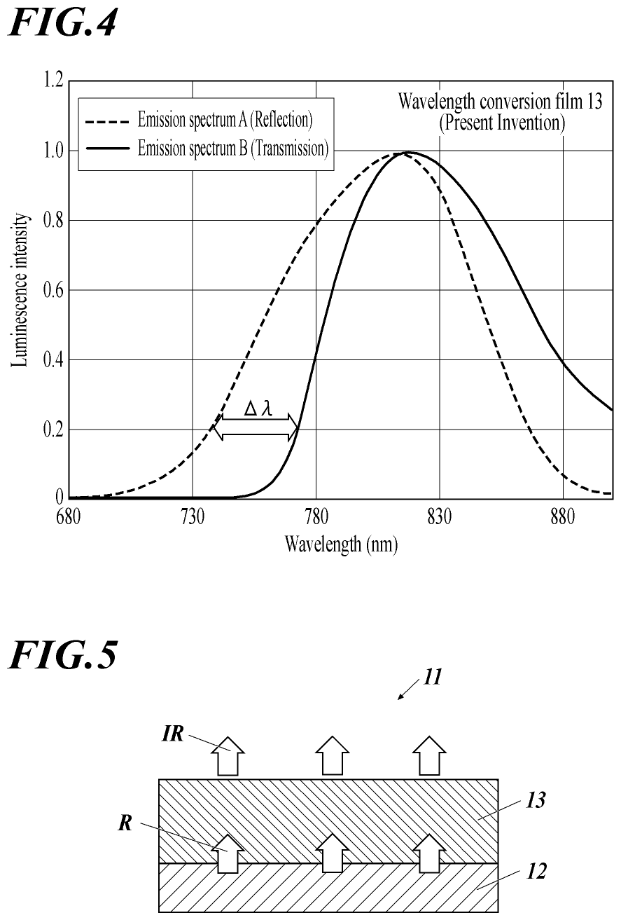

The emission spectrum A and the emission spectrum B measured by such a method are normalized as shown in FIG. 4, and a wavelength difference value Δλ at an intensity of 20% on the short-wave side of each emission spectrum is obtained, whereby a change width of a wavelength due to self-absorption of the wavelength conversion film may be obtained. The larger the wavelength difference value Δλ, the better the self-absorption characteristic and the better the characteristics of the wavelength conversion film may be determined.

### <<Configuration of Wavelength Conversion Film>>

The wavelength conversion film of the present invention is characterized in that it contains at least a luminescent dye, and more particularly, it is preferable to be formed of a luminescent dye and a transparent matrix resin.

Further, in the wavelength conversion film of the present invention, although there is no particular limitation on the maximum emission wavelength, in the case of application to a biometric authentication device, or a biometric device, it is preferable to have light emission in the region of the “biological window (650 to 1000 nm)”. It is more preferable that the maximum emission wavelength is 750 nm or more, which is a near-infrared region, and further, it is particularly preferable that the maximum emission wavelength is 800 nm or more, from the viewpoint of improving the biological transparency of light.

The wavelength conversion film of the present invention is disposed on a light source, for example, a surface light source, and constitutes a light-emitting member. If necessary, a cut filter for excluding light emitted without wavelength conversion may be laminated or included.

### [Luminescent Dye]

In the luminescent dye according to the present invention, it is preferable that wavelength difference value Δλ at an intensity of 20% on the short-wave side of each of the above-described emission spectrum A and emission spectrum B is 10 nm or more.

The luminescent dye applicable to the present invention is not specifically limited as long as it satisfies a condition in which the product value (ε×C) of the molar extinction coefficient ε and the luminescent dye amount C (mol) specified in the present invention is 1.0×10⁻³ or more. Examples thereof include complexes such as deuterated tris(hexafluoroacetylacetonato)neodymium (III): Nd(hfa-D₃), and pyrromethene-based boron, pyrrolopyrrole cyanine dye, inorganic nanoparticles containing ions such as rare earths, quantum dot nanoparticles composed of indium arsenic and lead sulfide, water-soluble silicon nanoparticles, metal complexes (gold, silver, copper, platinum, iridium, and ruthenium), and further, luminescent dyes such as squarylium, cyanine, phthalocyanine, croconium rhodamine, eosin, fluorescein, triphenylmethane, porphyrin, perylene, coumarin, thiazoloquinoxaline, benzobisthiadiazole, and thiophene, and luminescent polymers may be used, but the present invention is not limited to these.

Further, among these luminescent dyes, pyrrolopyrrole cyanine dyes, squarylium compounds, and perylene compounds are preferable. Further, the squarylium compound has light emission in the near-infrared light region, and may obtain characteristics having high durability and excellent luminous efficiency. It may be particularly preferably used as a luminescent dye in the present invention.

### [Squarylium Compounds]

Hereinafter, details of the squarylium compound suitable for the present invention will be described.

### (Squarylium Compound Group 1: Squarylium Compounds Represented by Formula (1) and Formula (2))

As one example of the squarylium compound that may be suitably used in the present invention, squarylium compounds having a structure represented by the following Formula (1) or Formula (2) may be mentioned.

### <Compound Having a Structure Represented by Formula (1)>

The type of the squarylium compound according to the present invention is not particularly limited, but is preferably a compound having a structure represented by the following Formula (1).

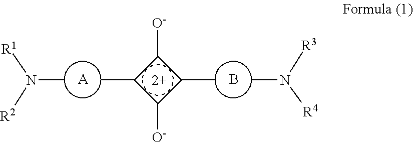

In Formula (1), A and B respectively represent an aromatic hydrocarbon ring which may have a substituent, or an aromatic heterocycle which may have a substituent, and adjacent substituents may be bonded to each other to form a ring. R¹ to R⁴ respectively represent a substituent, and at least one of R¹ to R⁴ has an aromatic hydrocarbon ring. Further, R¹ to R⁴ may be respectively bonded to each other to form a ring.

The aromatic hydrocarbon ring (it may be called as “an aromatic hydrocarbon ring group”, “an aromatic carbon ring group”, or “an aryl group”) represented by A and B is a substituted or unsubstituted aromatic hydrocarbon ring having 6 to 18 carbon atoms. Examples thereof are a phenyl group, a naphthyl group, an anthryl group, a fluorenyl group, a phenanthryl group, and a biphenylyl group. Preferably, a phenyl group, a naphthyl group, and an anthryl group may be mentioned. Examples of the aromatic heterocyclic group represented by A and B include a pyridyl group, a pyrimidinyl group, a furyl group, a pyrrolyl group, an imidazolyl group, a benzimidazolyl group, a pyrazolyl group, a pyrazinyl group, a triazolyl group (for example, 1,2,4-triazol-1-yl group, and 1,2,3-triazol-1-yl group), a pyrazolotriazolyl group, an oxazolyl group, a benzoxazolyl group, a thiazolyl group, an isoxazolyl group, an isothiazolyl group, a furazanyl group, a thienyl group, a quinolyl group, a benzofuryl group, a dibenzofuryl group, a benzothienyl group, a dibenzothienyl group, an indolyl group, a carbazolyl group, a carbolynyl group, a diazacarbazolyl group (indicating a ring structure in which one of the carbon atoms constituting the carboline ring of the carbolynyl group is replaced with a nitrogen atom), a quinoxalinyl group, a pyridazinyl group, a triazinyl group, a quinazolinyl group, and a phthalazinyl group.

Examples of the substituent represented by R¹ to R⁴ and the substituent that may be possessed by A and B in Formula (1) are as follows: an alkyl group (for example, a methyl group, an ethyl group, a propyl group, an isopropyl group, a tert-butyl group, a pentyl group, a hexyl group, an octyl group, a dodecyl group, a tridecyl group, a tetradecyl group, and a pentadecyl group); a cycloalkyl group (for example, a cyclopentyl group and a cyclohexyl group); an alkenyl group (for example, a vinyl group and an allyl group); an alkynyl group (for example, an ethynyl group and a propargyl group); an aromatic hydrocarbon group (for example, a phenyl group, a p-chlorophenyl group, a mesityl group, a tolyl group, a xylyl group, a naphthyl group, an anthryl group, an azulenyl group, an acenaphthenyl group, a fluorenyl group, a phenantolyl group, an indenyl group, a pyrenyl group, and a biphenylyl group): an aromatic heterocyclic group (for example, a pyridyl group, a pyrazyl group, a pyrimidinyl group, a triazyl a group, a furyl group, a pyrrolyl group, an imidazolyl group, a benzimidazolyl group, a pyrazolyl group, a pyrazinyl group, a triazolyl group (for example, 1,2,4-triazol-1-yl group, and 1,2,3-triazol-1-yl group), an oxazolyl group, a benzoxazolyl group, a thiazolyl group, an isoxazolyl group, an isothiazolyl group, a furazanyl group, a thienyl group, a quinolyl group, a benzofuryl group, a dibenzofuryl group, a benzothienyl group, a dibenzothienyl group, an indolyl group, a carbazolyl group, an diazacarbazolyl group (indicating a ring structure in which one of the carbon atoms constituting the carboline ring of the carbazolyl group is replaced with a nitrogen atom), a quinoxalinyl group, a pyidazinyl group, a triazinyl group, a quinazolinyl group, and a phthalazinyl group; a heterocyclic group (for example, a pyrrolidyl group, an imidazolidyl group, a morpholyl group, and an oxazolidyl group); an alkoxy group (for example, a methoxy group, an ethoxy group, a propyloxy group, a pentyloxy group, an hexyloxy group, an octyloxy group, and a dodecyloxy group); a cycloalkoxy group (for example, a cyclopentyloxy group and a cyclohexyloxy group); an aryloxy group (for example, a phenoxy group and a naphthyloxy group); an alkylthio group (for example, a methylthio group, an ethylthio group, a propylthio group, a pentylthio group, hexylthio group, an octylthio group, and a dodecylthio group); a cycloalkylthio group (for example, a cyclopentylthio group and a cyclohexylthio group); an arylthio group (for example, a phenylthio group and a naphlhylthio group); an alkoxycarbonyl group (for example, a methyloxycarbonyl group, an ethyloxycarbonyl group, a butyloxycarbonyl group, an octyloxycarbonyl group, and a dodecyloxycarbonyl group); an aryloxycarbonyl group (for example, a phenyloxycarbonyl group and a naphthyloxycarbonyl group); a sulfamoyl group (for example, an aminosulfonyl group, a methylaminosulfonyl group, a dimethylamninosulfonyl group, a butylaminosulfonyl group, a hexylaminosulfonyl group, a cyclohexylaminosulfonyl group, an octylaminosulfonyl group, a dodecylaminosulfonyl group, a phenylaminosulfonyl group, a naphthylaminosulfonyl group, and a 2-pyridylaminosulfonyl group); an acyl group (for example, an acetyl group, an ethyl carbonyl group, a propylcarbonyl group, a pentylcarbonyl group, a cyclohexylcarbonyl group, an octylcarbonyl group, a 2-ethylhexylcarbonyl group, a dodecylcarbonyl group, a phenylcarbonyl group, a naphthylcarbonyl group, and a pyridylcarbonyl group); an acyloxy group (for example, an acetyloxy group, an ethylcarbonyloxy group, a butylcarbonyloxy group, an octylcarbonyloxy group, a dodecylcarbonyloxy group, and a phenylcarbonyloxy group); an amido group (for example, a methylcarbonylamino group, an ethylcarbonylamino group, a dimethylcarbonylamino group, a propylcarbonylamino group, a pentylcarbonylamino group, a cyclohexylcarbonylamino group, a 2-ethyhexylcarbonylamino group, an octylcarbonylamino group, a dodecylcarbonylamino group, a phenylcarbonylamino group, and a naphthylcarbonylamino group); a carbamoyl group (for example, an aminocarbonyl group, a methylaminocarbonyl group, a dimethylaminocarbonyl group, a propylaminocarbonyl group, a pentylaminocarbonyl group, a cyclohexylaminocarbonyl group, an octylaminocarbonyl group, a 2-ethymexylaminocarbonyl group, a dodecylaminocarbonyl group, a phenylaminocarbonyl group, a naphthylaminocarbonyl group, and a 2-pyridylaminocarbonyl group); a ureido group (for example, a methylureido group, an ethylureido group, a pentylureido group, a cyclohexylureido group, an octylureido group, a dodecylureido group, a phenylureido group, a naphthylureido group, and a 2-pyridylaminoureido group); a sulfinyl group (for example, a methylsulfinyl group, an ethylsufinyl group, a butylsulfinyl group, a cyclohexylsulfinyl group, a 2-ethylhexylsulfinyl group, a dodecylsulfinyl group, a phenylsulfinyl group, a naphthylsulfinyl group, and a 2-pyridylsulfinyl group); an alkylsulfonyl group (for example, a methylsulfonyl group, an ethylsulfonyl group, a butylsulfinyl group, a cyclohexylsulfonyl group, a 2-ethylhexylsulfonyl group, and a dodecylsulfonyl group); an arylsulfonyl group or a heteroarylsulfonyl group (for example, a phenylsulfonyl group, a naphthylsulfonyl group, and a 2-pyridylsulfonyl group); an amino group (for example, an amino group, an ethylamino group, a dimethylamino group, a butylamino group, a cyclopentylamino group, a dodecylamino group, an anilino group, a naphthylamino group, and a 2-pyridylamino group); a halogen atom (for example, a fluorine atom, a chlorine atom and a bromine atom); a fluorinated hydrocarbon group (for example, a fluoromethyl group, trifluoromethyl group, a pentafluoroethyl group and a pentafluorophenyl group), a cyano group; a nitro group; a hydroxy group; a mercapto group; a silyl group (for example, a trimethylsilyl group, a triisopropylsilyl group, a triphenylsilyl group, and a phenyldiethylsilyl group) and a phosphono group. Preferred examples include an alkyl group, an aromatic hydrocarbon group, an amino group, a hydroxy group and a silyl group.

Further, these substituents may be further substituted by the above-mentioned substituents.

Of these, the substituent represented by R¹ to R⁴ is preferably an alkyl group or an aromatic hydrocarbon group.

As the alkyl group represented by R¹ to R⁴, a substituted or unsubstituted alkyl group having 6 to 10 carbon atoms is preferable. For example, a methyl group, an ethyl group, a propyl group, an isopropyl group, a butyl group, an isobutyl group, a pentyl group, a hexyl group, an octyl group, a dodecyl group, a cyclopentyl group, and a cyclohexyl group may be mentioned.

As the aromatic hydrocarbon group represented by R¹ to R⁴, a substituted or unsubstituted aromatic hydrocarbon group having 6 to 18 carbon atoms is preferable. Examples thereof are a phenyl group, a naphthyl group, an anthryl group, a fluorenyl group, a phenanthryl group, and a biphenylyl group. Preferably, a phenyl group and a naphthlyl group may be mentioned.

The cyclic structure formed by the adjacent substituents may be an aromatic ring or an alicyclic ring, it may contain a hetero atom, and the cyclic structure may be a fused ring having two or more rings. The hetero atom referred to here is preferably one selected from the group consisting of a nitrogen atom, an oxygen atom and a sulfur atom. Examples of the formed ring structure include a benzene ring, a naphthalene ring, a pyridine ring, a pyridazine ring, a pyrimidine ring, a pyrazine ring, a pyrrole ring, an imidazole ring, a pyrazole ring, a triazole ring, an imidazoline ring, an oxazole ring, an isooxazole ring, a thiazole ring, an isothiazole ring, a cyclohexadiene ring, a cyclohexene ring, a cyclopentaene ring, a cycloheptatriene ring, a cycloheptadiene ring, a cycloheptaene ring, a carbazole ring, and a dibenzofuran ring.

### <Compound Having a Structure Represented by Formula (A)>

In Formula (1), it is preferable that A, B, or both of A and B have a hydroxy group from the viewpoint of improving luminescence. From the viewpoint of expanding the π-conjugation in the molecule and lengthening the maximum emission wavelength, it is more preferable to be represented by the following Formula (A).

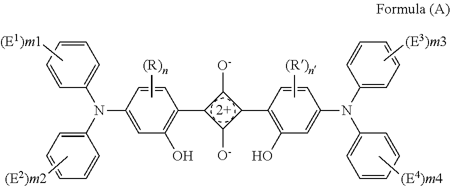

In Formula (A), E¹ to E⁴ respectively represent a substituent, m¹ to m⁴ respectively represent 0 or an integer of 1 to 5. R and R′ respectively represent a substituent, and n and n′ respectively represent 0 or an integer of 1 to 3, provided that when m1 to m4, n and n′ are 2 or more, a plurality of E¹ to E⁴, and R and R′ may be the same or different.

The structure of the substituent represented by E¹ to E⁴, R and R′ is synonymous with the substituent of Formula (1) described in detail above. In particular, the substituents represented by E¹ to E⁴ are preferably alkyl groups such as a methyl group, an ethyl group and a t-butyl group, and the substituents represented by R and R′ are preferably a hydrogen atom or a hydroxy group.

The squarylium compound according to the present invention may be synthesized with the methods described in Chemistry of Materials, Vol. 23, p. 4789 (2011) and The Journal of Physical Chemistry, Vol. 91, p 5184 (1987), for example. Or it may be synthesized by referring to the methods described in the references published in these documents.

The following are representative examples of the luminescent dye, the squarylium compound, the compound having the structure represented by Formula (1), and the compound having the structure represented by Formula (A) contained in the wavelength conversion film according to the present invention. However, the present invention is not limited to these.

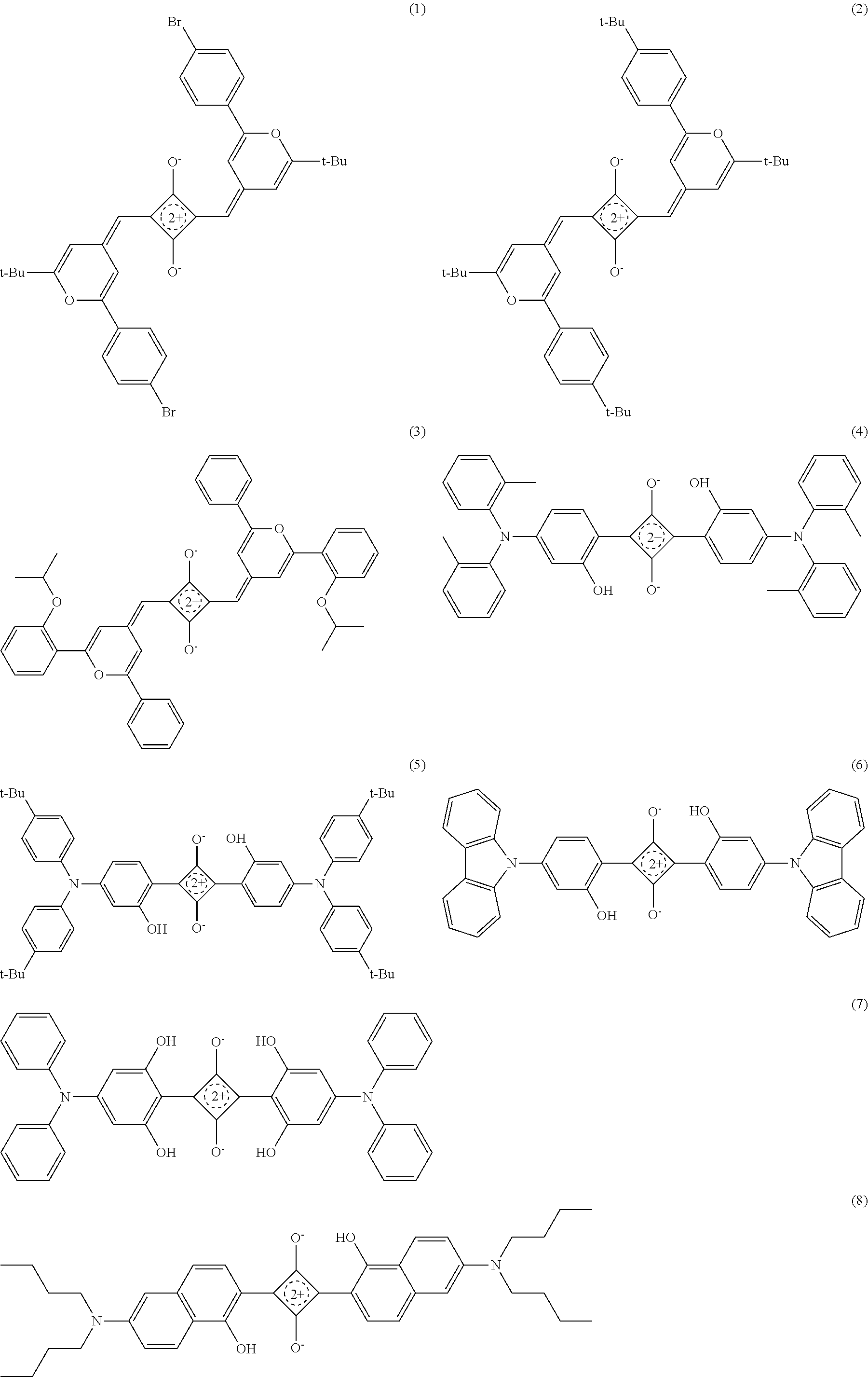

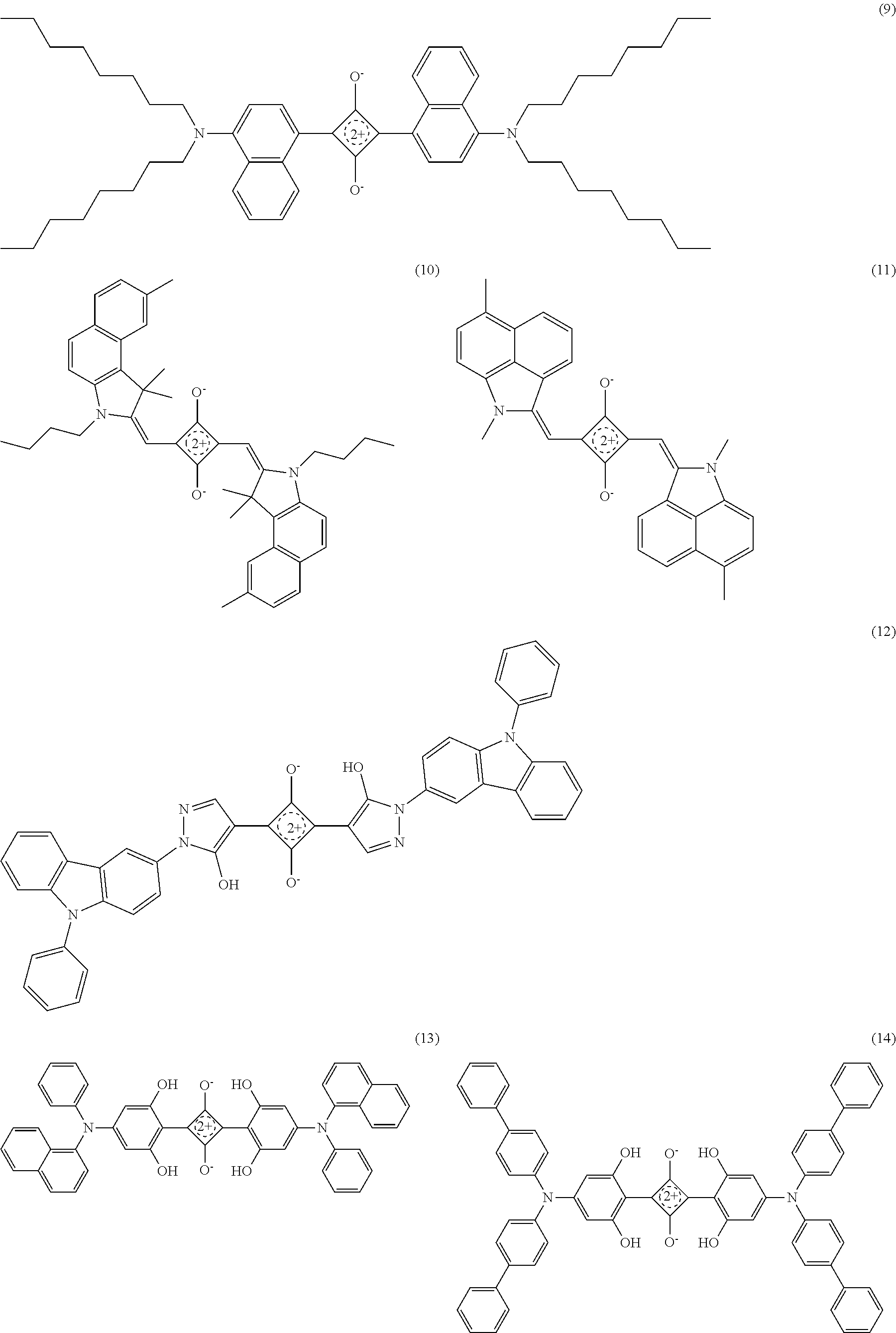

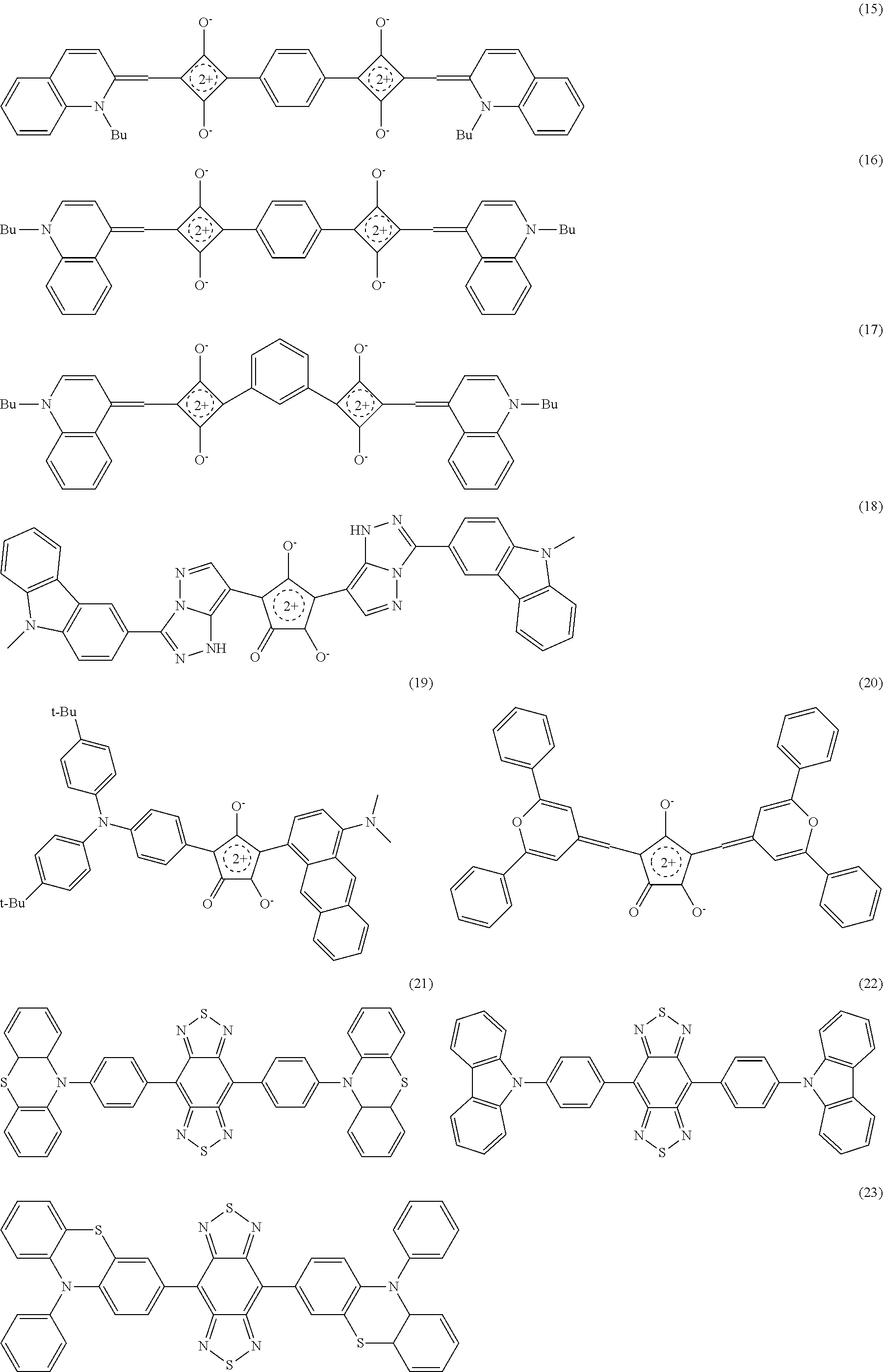

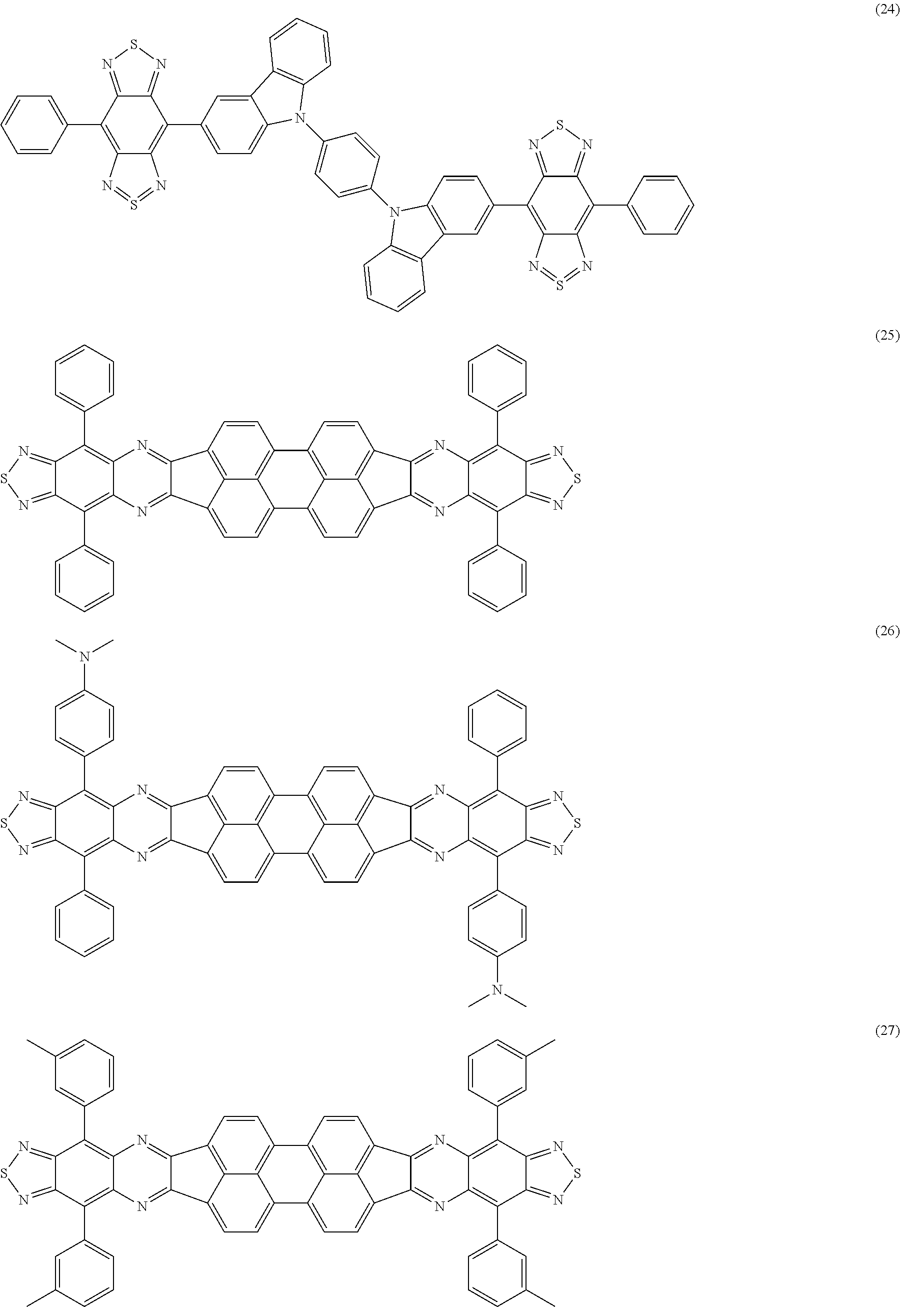

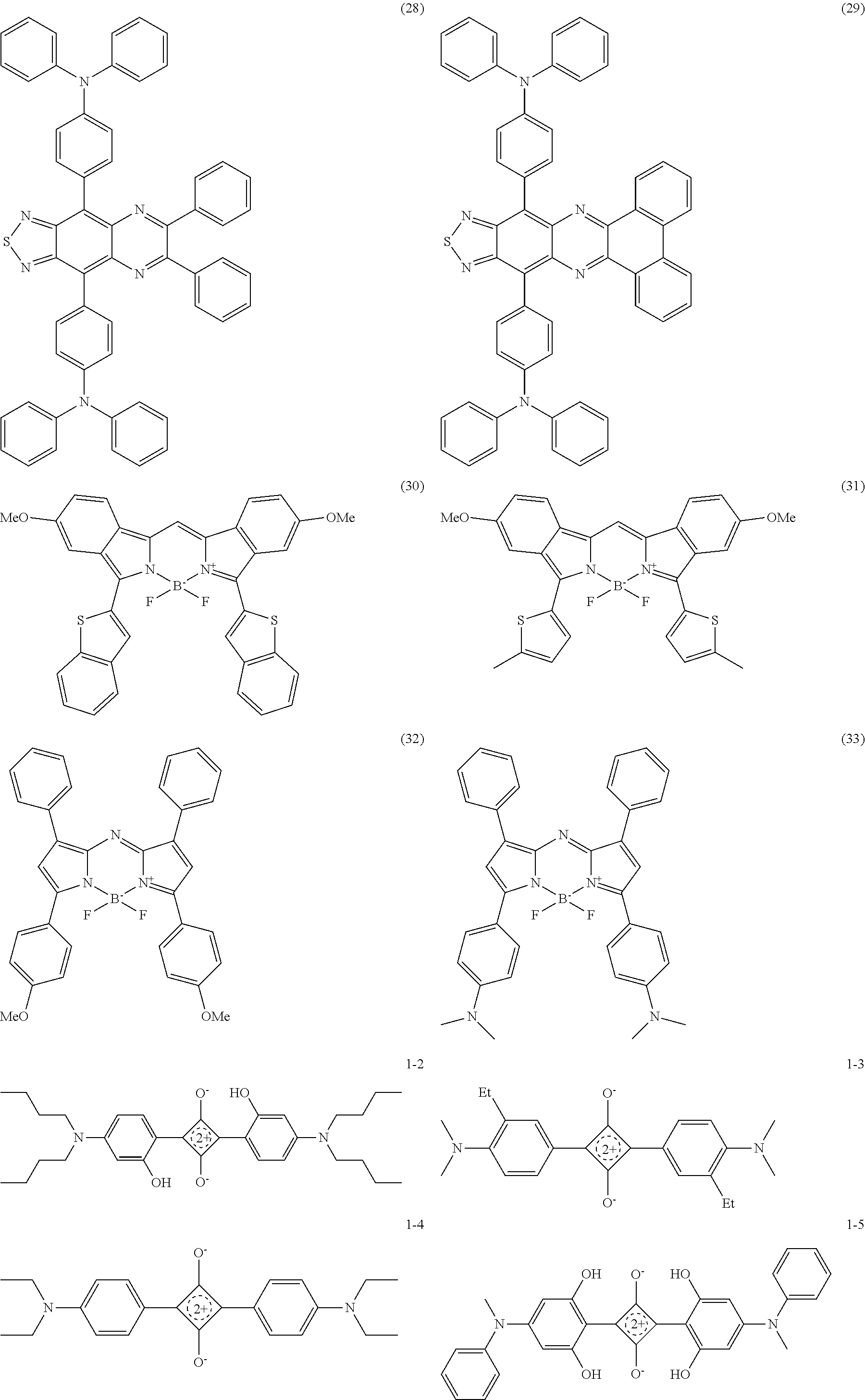

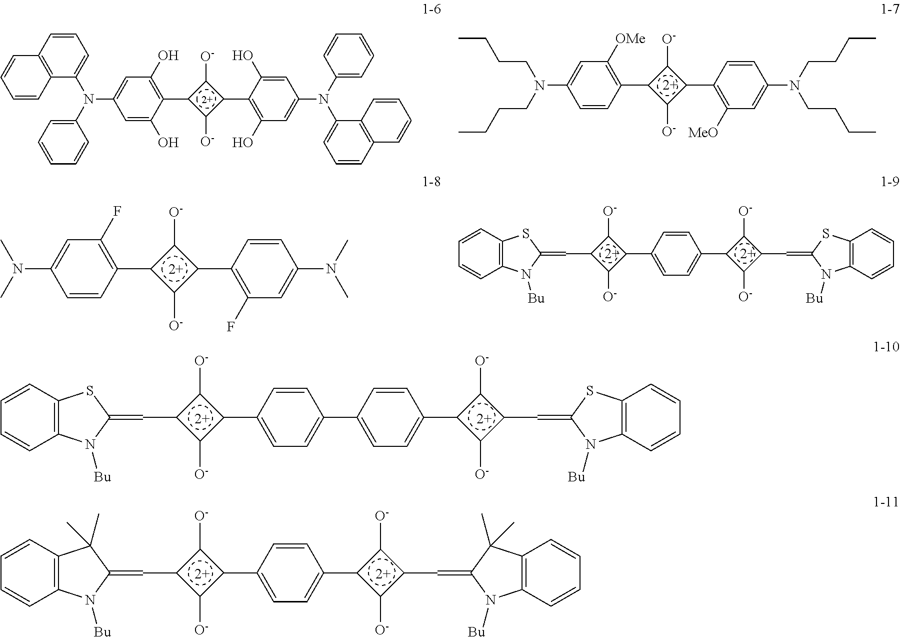

### (Squarylium Compound Group 2: Squarylium Compounds Represented by Formula (2))

In the present invention, a squarylium compound having a structure represented by the following Formula (2) may also be used as a luminescent dye.

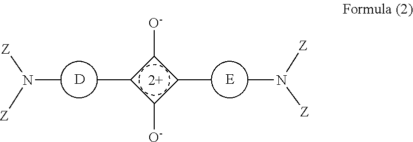

In the above Formula (2), ring D and ring E each independently represent an aryl ring which nay be substituted with a hydroxy group or an alkoxy group.

Z represents a group of the following Formula (Z) or a substituent, and at least one of Zs represents the group of Formula (Z).

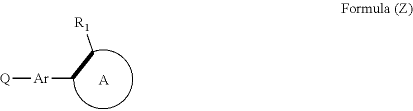

In Formula (Z), ring A represents an aryl ring, a heteroaryl ring or a fused ring thereof. Q represents a binding site with Ar in a luminescent residue. R₁ represents a group in which a dihedral angle between Ar and ring A is 45 degree or more. A thick lime represents a single bond or a double bond.

Further, as the luminescent dye, a squarylium compound having a structure represented by the following Formula (2b) may also be used.

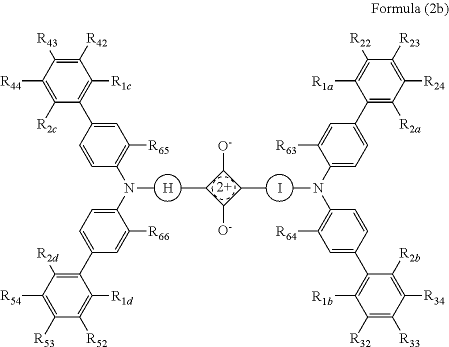

In Formula (2b), R₁ₐ to R_{1d}, R₂ₐ, to R_{2d}, R₂₂ to R₂₄, R₃₂ to R₃₄, R₄₂ to R₄₄, R₅₂ to R₅₄, and R₆₃ to R₆₆ each independently represent a hydrogen atom, an alkyl group, an alkoxy group, a phenoxy group, an amino group, an aryl group, or a heteroaryl group, but at least one of the combinations of R₁ₐ and R₂ₐ, R_{1b} and R_{2b}, R_{1c} and R_{2c}, and R_{1d} and R_{2d} does not both represent a hydrogen atom.

R₁ₐ and R₂₂, R₂ₐ and R₂₄, R_{1b} and R₃₂, R_{2b} and R₃₄, R_{1c} and R₄₂, R_{2c} and R₄₄, R_{1d} and R₅₂, and R_{2d} and R₅₄ may be bonded to form a ring structure.

Ring H and ring I each independently represent an aryl group which may be substituted with a hydroxy group or an alkoxy group.

In the squarylium compound having a structure represented by Formula (2b), it is preferable that R₁ₐ to R_{1d} and R₂ₐ to R_{2d} are the same and represent an alkyl group, an alkoxy group, a phenoxy group, an amino group, an aryl group or a heteroaryl group.

In the squarylium compound having a structure represented by Formula (2b), it is preferable that ring H and ring I are the same and represent an aryl group having a hydroxyl group, R₂₃, R₃₃, R₄₃ and R₅₃ are the same as R₁ₐ to R_{1d} and R₂ₐ to R_{2d}, respectively, or represent a hydrogen atom, but at least one of the combinations of R₁ₐ and R₂ₐ, R_{1b} and R_{2d}, R_{1c} and R_{2c}, and R_{1d} and R_{2d} does not both represent a hydrogen atom, and R₂₂, R₂₄, R₃₂, R₃₄, R₄₂, R₄₄, R₅₂ and R₅₄ represent a hydrogen atom.

Further, it is preferable that ring H and ring I are the same and represent an aryl group having a hydroxy group; and R₁ₐ is bonded to R₂₂, R₂ₐ is bonded to R₂₄, R_{1b} is bonded to R₃₂, R_{2b} is bonded to R₃₄, R_{1c} is bonded to R₄₂, R_{2c} is bonded to R₄₄, R_{1d} is bonded to R₅₂, and R₂₄ is bonded to R₅₄ respectively to form a 5-membered ring or a 6-membered ring with a carbon chain.

Further, in the present invention, as the compound used as the dye, the pyrrolopyrrole cyanine dye described in Document 1 and the dicyanovinyl-substituted squarylium dye described in Document 2 described below may also be preferably used.

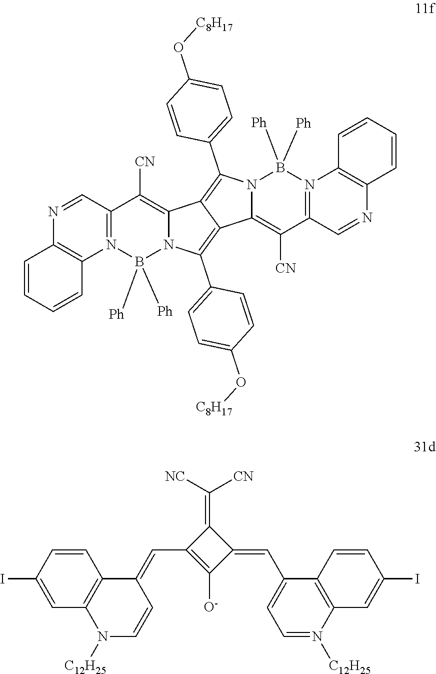

Document 1: Fischer et al., Angew. Clem. Int. Ed. 2007, 46, 3750-3753

Document 2: Mayerhoffer et al., Chem. Eur. J. 2013, 19, 218-232

Further, as the compound used in the present invention, compounds (1a) to (1d) and compounds (4) to (11) having structures represented by the following Formulas (1a) to (1d) and Formulas (4) to (11) are also suitably used. Hereinafter, each compound will be exemplified together with a description of each of Formulas (1a) to (1d) and Formulas (4) to (11).

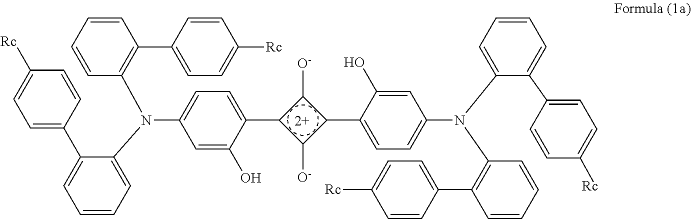

In Formula (1a), each Rc independently represents a hydrogen atom or a substituent, and at least one of the four Rcs represents a straight or branched alkyl group (a) having 4 to 12 carbon atoms which may have —O— as a linking group, or a group (P) having a structure represented by the following Formula (P).

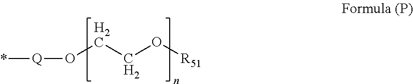

In Formula (P), an asterisk (*) represents a substitution site. Q represents a single bond or a divalent linking group. n represents an integer of 1 to 9. R₅₁ represents a hydrogen atom or a methyl group.

In the compound (1a) having the structure represented by the above Formula (1a), examples of the compound in which all 4 Rcs are the same alkyl group (a) or group (P) are shown in Table I. Table I listed the compound name and the type of Rc that are possessed by the compound. Note that, in Table I, only abbreviations are shown for compound names. Similarly, in the table exemplifying other compounds shown below, only abbreviations are shown for compound names.

In Table I, “n-butyl” represents an n-butyl group, “n-pentyl” represents an n-pentyl group, “n-hexyl” represents an n-hexyl group, “n-heptyl” represents an n-heptyl group. “n-octyl” represents an n-octyl group. “n-nonyl” represents an n-nonyl group, “n-decyl” represents an n-decyl group. “n-undecyl” represents an n-undecyl group, and “n-dodecyl” represents an n-dodecyl group. The group (a1) is denoted by (a1). The group (P) is denoted by (P) and the type of Q, the number of n, and the type of R 11 are shown in parentheses after “(P)”, e.g., (P) (Q=—CH₂—, n=2, R₁₁=CH₃). However, when Q is a single bond, the notation of Q is omitted. The same applies to the description of the table relating to other compounds described later.

In addition, in each of the tables shown below, the group (a1) is a 1-propylbutyl group, the group (a2) is a 1-ethylpentyl group, the group (a3) is a 2,4,4-trimethylpentyl group, the group (a4) is an isobutyl group, the group (a5) is a 2-ethylbutyl group, the group (a6) is a 2-ethylhexyl group, and the group (a7) is a 2-butyloctyl group.

TABLE I

| Compound name | |
|---|---|
| (Abbreviation) | Rc |
| (1a)-1 | n-buty |
| (1a)-2 | n-pentyl |
| (1a)-3 | n-hexyl |
| (1a)-4 | n-heptyl |
| (1a)-5 | n-octyl |
| (1a)-6 | n-nonyl |
| (1a)-7 | n-decyl |
| (1a)-8 | n-undecyl |
| (1a)-9 | n-dodecyl |
| (1a)-10 | (a4) |
| (1a)-11 | (a5) |
| (1a)-12 | (a6) |
| (1a)-13 | (a7) |
| (1a)-14 | (P)(n = 2, R₅₁═CH₃₎ |
| (1a)-15 | (P)(n = 3, R₅₁═CH₃₎ |
| (1a)-16 | (P)(n = 4, R₅₁═CH₃₎ |
| (1a)-17 | (P)(n = 5, R₅₁═CH₃₎ |
| (1a)-18 | (P)(n = 6, R₅₁═CH₃₎ |
| (1a)-19 | (P)(n = 7, R₅₁═CH₃₎ |
| (1a)-20 | (P)(n = 8, R₅₁═CH₃₎ |
| (1a)-21 | (P)(n = 9, R₅₁═CH₃₎ |
| (1a)-22 | (P)(n = 2, R₅₁═H) |
| (1a)-23 | (P)(n = 3, R₅₁═H) |
| (1a)-24 | (P)(n = 4, R₅₁═H) |
| (1a)-25 | (P)(n = 5, R₅₁═H) |
| (1a)-26 | (P)(n = 6, R₅₁═H) |
| (1a)-27 | (P)(n = 7, R₅₁═H) |
| (1a)-28 | (P)(n = 8, R₅₁═H) |
| (1a)-29 | (P)(n = 9, R₅₁═H) |
| (1a)-30 | (P)(Q = —CH₂—, n = 2, R₅₁═CH₃) |
| (1a)-31 | (P)(Q = —CH₂—, n = 3, R₅₁═CH₃) |
| (1a)-32 | (P)(Q = —CH₂—, n = 4, R₅₁═CH₃) |
| (1a)-33 | (P)(Q = —CH₂—, n = 8, R₅₁═CH₃) |
| (1a)-34 | (P)(Q = —C(═O)—, n = 2, R₅₁═CH₃) |
| (1a)-35 | (P)(Q = —C(═O)—, n = 3, R₅₁═CH₃) |
| (1a)-36 | (P)(Q = —C(═O)—, n = 4, R₅₁═CH₃) |
| (1a)-37 | (P)(Q = —C(═O)—, n = 8, R₅₁═CH₃) |
| (1a)-38 | —O—n-butyl |

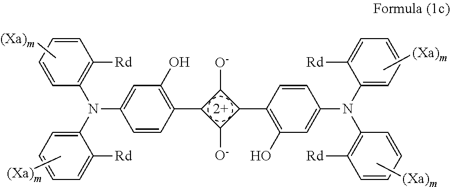

In Formula (1c), each Rd independently represents a hydrogen atom or a substituent, and at least one of 4 Rds represents a linear or branched alkyl group (a) having 4 to 12 carbon atoms which may have —O— as a linking group, or a group (P) having a structure represented by the above Formula (P). Each Xa independently represents an alkyl group or an alkoxy group having 1 to 3 carbon atoms or a phenyl group. m represents an integer of 0 to 4.

Examples of the compounds (1c) having a structure represented by Formula (1c) in which 4 (Xa)ms are all the same (in the benzene ring to which Rd and (Xa)ms are bonded, each (Xa)m is the same) are shown in Table II. In addition, all 4 Rds are the same alkyl group (a) or group (P). In Table II, the compound name and the type of Rd possessed by the compound, a group or an atom bonded to a carbon atom other than the carbon atom to which Rd is bonded are indicated by X11, X12, X13, and X14 in order from next to Rd. In Table II, “—O-(a2)” indicates a group (a2) to which —O— is bonded as a linking group. In addition, “2,4,6-trimethoxyphenyl” represents a 2,4,6-trimethoxyphenyl group, “Me” represents a methyl group, and “Ph” represents a phenyl group, respectively. The same applies to the description of the table relating to other compounds described later.

TABLE II

| Compomd name | | | | | |
|---|---|---|---|---|---|
| (Abbreviation) | Rd | XII | X12 | X13 | X14 |
| (1c)-1 | n-butyl | H | H | H | H |
| (1c)-2 | n-pentyl | H | H | H | H |
| (1c)-3 | n-hexyl | H | H | H | H |
| (1c)-4 | n-heptyl | H | H | H | H |
| (1c)-5 | n-octyl | H | H | H | H |
| (1c)-6 | n-nonyl | H | H | H | H |
| (1c)-7 | n-decyl | H | H | H | H |
| (1c)-8 | n-undecyl | H | H | H | H |
| (1c)-9 | n-dodecyl | H | H | H | H |
| (1c)-10 | (a4) | H | H | H | H |
| (1c)-11 | (a5) | H | H | H | H |
| (1c)-12 | (a6) | H | H | H | H |
| (1c)-13 | (a7) | H | H | H | H |
| (1c)-14 | (P)(n = 2, R₅₁═CH₃) | H | H | H | H |
| (1c)-15 | (P)(n = 3, R₅₁═CH₃) | H | H | H | H |
| (1c)-16 | (P)(n = 4, R₅₁═CH₃) | H | H | H | H |
| (1c)-17 | (P)(n = 5, R₅₁═CH₃) | H | H | H | H |
| (1c)-18 | (P)(n = 6, R₅₁═CH₃) | H | H | H | H |
| (1c)-19 | (P)(n = 7, R₅₁═CH₃) | H | H | H | H |
| (1c)-20 | (P)(n = 8, R₅₁═CH₃) | H | H | H | H |
| (1c)-21 | (P)(n = 9, R₅₁═CH₃) | H | -; | H | |
| (1c)-22 | (P)(n = 2, R₅₁═H) | H | H | H | H |
| (1c)-23 | (P)(n = 3, R₅₁═H) | H | H | H | H |
| (1c)-24 | (P)(n = 4, R₅₁═H) | H | H | H | H |
| (1c)-25 | (P)(n = 5, R₅₁═H) | H | H | H | H |
| (1c)-26 | (P)(n = 6, R₅₁═H) | H | H | H | il |
| (1c)-27 | (P)(n = 7, R₅₁═H) | H | H | H | H |
| (1c)-28 | (P)(n = 8, R₅₁═H) | H | H | H | H |
| (1c)-29 | (P)(n = 9, R₅₁═H) | H | H | H | H |
| (1c)-30 | (P)(Q = —CH₂—, n = 2, R₅₁═CH₃) | H | H | H | H |
| (1c)-31 | (P)(Q = —CH₂—, n = 3, R₅₁═CH₃) | H | H | H | H |
| (1c)-32 | (P)(Q = —CH₂—, n = 4, R₅₁═CH₃) | H | H | H | H |
| (1c)-33 | (P)(Q = —CH₂—, n = 8, R₅₁═CH₃) | H | H | H | H |
| (1c)-34 | (P)(Q = —C(═O)—, n = 2, R₅₁═CH₃) | H | H | H | H |
| (1c)-35 | (P)(Q = —C(═O)—, n = 3, R₅₁═CH₃) | H | H | H | H |
| (1c)-36 | (P)(Q = —C(═O)—, n = 4, R₅₁═CH₃) | H | H | H | H |
| (1c)-37 | (P)(Q = —C(═O)—, n = 8, R₅₁═CH₃) | H | H | H | H |
| (1c)-38 | n-butyl | Me | H | H | H |
| (1c)-39 | n-butyl | H | Me | H | H |
| (1c)-40 | n-butyl | H | H | Me | H |
| (1c)-41 | n-butyl | H | Ph | H | H |
| (1c)-42 | —O— (a2) | H | 2,4,6-trimethoxyphenyl | H | H |

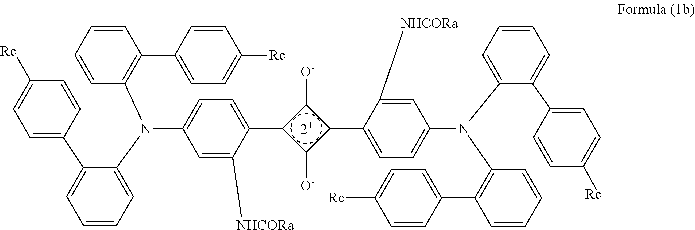

In Formula (1b), each Ra independently represents a hydrocarbon group. Each Rc independently represents a hydrogen atom or a substituent, and at least 1 of 4 Rcs represents a linear or branched alkyl group (a) having 4 to 12 carbon atoms which may have —O— as a linking group, or a group (P) having a structure represented by the above Formula (P)).

In the compound (1b) having the structure represented by the above Formula (1b), examples of the compound are shown in Table III, wherein 2 Ras are the same alkyl group and all 4 Rcs are the same alkyl group (a) or group (P). Table III lists the compound names and the types of Rc and Ra that are possessed by the compound.

TABLE III

| Compound name | | |
|---|---|---|
| (Abbreviation) | Rc | Ra |
| (1b)-1 | n-butyl | n-butyl |
| (1b)-2 | n-pentyl | n-pentyl |
| (1b)-3 | n-hexyl | n-hexyl |
| (1b)-4 | n-heptyl | n-heptyl |
| (1b)-5 | n-octyl | n-octyl |
| (1b)-6 | n-nonyl | n-nonyl |
| (1b)-7 | n-decyl | n-decyl |
| (1b)-8 | n-undecyl | n-undecyl |
| (1b)-9 | n-dodecyl | n-dodecyl |
| (1b)-10 | (a4) | (a4) |
| (1b)-11 | (a5) | (a1) |
| (1b)-12 | (a6) | (a2) |
| (1b)-13 | (a7) | (a3) |
| (1b)-14 | n-butyl | Me |
| (1b)-15 | n-hexyl | Me |
| (1b)-16 | (a4) | Me |
| (1b)-17 | (a5) | Me |
| (1b)-19 | (a6) | Me |
| (1b)-19 | (P)(n = 2, R₅₁═CH₃) | Me |
| (1b)-20 | (P)(n = 3, R₅₁═CH₃) | Me |
| (1b)-21 | (P)(n = 4, R₅₁═CH₃) | Me |
| (1b)-22 | (P)(n = 5, R₅₁═CH₃) | Me |
| (1b)-23 | (P)(n = 6, R₅₁═CH₃) | Me |
| (1b)-24 | (P)(n = 7, R₅₁═CH₃) | Me |
| (1b)-25 | (P)(n = 8, R₅₁═CH₃) | Me |
| (1b)-26 | (P)(n = 9, R₅₁═CH₃) | Me |
| (1b)-27 | (P)(n = 2, R₅₁═H) | Me |
| (1b)-28 | (P)(n = 3, R₅₁═H) | Me |
| (1b)-29 | (P)(n = 4, R₅₁═H) | Mo |
| (1b)-30 | (P)(n = 5, R₅₁═H) | Me |
| (1b)-31 | (P)(n = 6, R₅₁═H) | Me |
| (1b)-32 | (P)(n = 7, R₅₁═H) | Me |
| (1b)-33 | (P)(n = 8, R₅₁═H) | Me |
| (1b)-34 | (P)(n = 9, R₅₁═H) | Me |
| (1b)-35 | (P)(Q = —CH₂—, n = 2, R₅₁═CH₃) | Me |
| (1b)-36 | (P)(Q = —CH₂—, n = 3, R₅₁═CH₃) | Me |
| (1b)-37 | (P)(Q = —CH₂—, n = 4, R₅₁═CH₃) | Me |
| (1b)-38 | (P)(Q = —CH₂—, n = 8, R₅₁═CH₃) | Me |
| (1b)-39 | (P)(Q = —C(═O)—, n = 2, R₅₁═CH₃) | Me |
| (1b)-40 | (P)(Q = —C(═O)—, n = 3, R₅₁═CH₃) | Me |
| (1b)-41 | (P)(Q = —C(═O)—, n = 4, R₅₁═CH₃) | Me |
| (1b)-42 | (P)(Q = —C(═O)—, n = 8, R₅₁═CH₃) | Me |
| (1b)-43 | —O—n-butyl | Me |

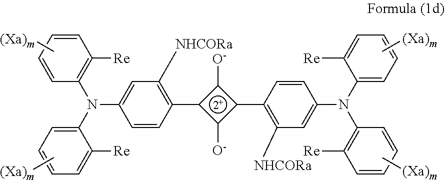

In Formula (1d), each Ra independently represents a hydrocarbon group. Each Re independently represents a hydrogen atom or a substituent. Each Xa independently represents an alkyl group having 1 to 3 carbon atoms or a substituted or unsubstituted phenyl group, and when bonded to the 4 position, Xa may be a group having a structure represented by the above Formula (D). m represents an integer of 0 to 4. However, in Formula (1d), any one of Ras represents a linear or branched alkyl group (a) having 4 to 12 carbon atoms, or at least one of 4 Res represents a linear or branched alkyl group (a) laving 4 to 12 carbon atoms which may have —O— as a linking group, or a group (P) having a structure represented by Formula (P), alternatively, a group having a structure represented by Formula (D) is bonded to the 4 position as Xa.

Examples of the compound in which 2 Ras, 4 Res, and 4 (Xa)ms are all the same in the compound (1d) having the structure represented by the above Formula (1d) are shown in Table IV. 2 Ras are a methyl group, an ethyl group (indicated by “Et” in the table) or an alkyl group (a). All 4 Res are the same alkyl group (a), group (P), methyl group or phenyl group. In Table IV, the compound name and the type of Ra and Re that are possessed by the compound, a group or an atom bonded to a carbon atom other than the carbon atom to which Re is bonded are indicated by X11, X12, X13, and X14 in order from next to Re. Note that X12 corresponds to the 4 position in the benzene ring of the group (A). “(D)-1” in Table IV indicates a group having a structure represented by the following formula.

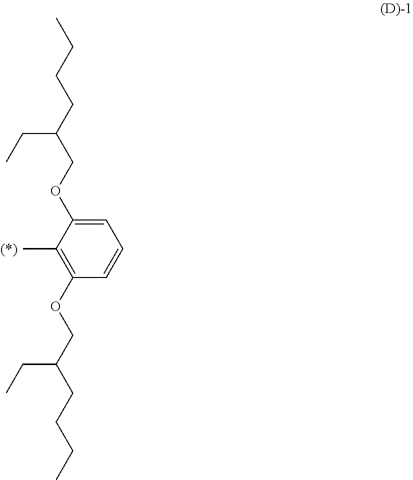 [SMILES: CCCCC(CC)COc1cccc(OCC(CC)CCCC)c1C]

TABLE IV

| Compound | | | | | | |
|---|---|---|---|---|---|---|
| name | | | | | | |
| (Abbreviation) | Ra | Re | X11 | X12 | X13 | X14 |
| (1d)-1 | n-butyl | Me | H | H | H | H |
| (1d)-2 | n-pentyl | Me | H | H | H | H |
| (1d)-3 | n-hexyl | Me | H | H | H | H |
| (1d)-4 | n-heptyl | Me | H | H | H | H |
| (1d)-5 | n-octyl | Me | H | H | H | H |
| (1d)-6 | n-nonyl | Me | H | H | H | H |
| (1d)-7 | n-decyl | Me | H | H | H | H |
| (1d)-8 | n-undecyl | Me | H | H | H | H |
| (1d)-9 | n-dedecyl | Me | H | H | H | H |
| (1d)-10 | (a4) | Me | H | H | H | H |
| (1d)-11 | (a1) | Me | H | H | H | H |
| (1d)-12 | (a2) | Me | H | H | H | H |
| (1d)-13 | (a3) | Me | H | H | H | H |
| (1d)-14 | Me | n-butyl | H | H | H | H |
| (1d)-15 | Et | n-pentyl | H | H | H | H |
| (1d)-16 | Et | n-hexyl | H | H | H | H |
| (1d)-17 | Me | n-heptyl | H | H | H | H |
| (1d)-18 | Me | n-octyl | H | H | H | H |
| (1d)-19 | Me | n-nonyl | H | H | H | H |
| (1d)-20 | Me | n-decyl | H | H | H | H |
| (1d)-21 | Me | n-undecyl | H | H | H | H |
| (1d)-22 | Me | n-dodecyl | H | H | H | H |
| (1d)-23 | Me | (P)(Q = —CH₂—, n = 2, R₅₁ = | H | H | H | H |
| | | CH₃) | | | | |
| (1d)-24 | Me | (P)(Q = —CH₂—, n = 3, R₅₁ = | H | H | H | H |
| | | CH₃) | | | | |
| (1d)-25 | Me | (P)(Q = —CH₂—, n = 4, R₅₁ = | H | H | H | H |
| | | CH₃) | | | | |
| (1d)-26 | Me | (P)(Q = —CH₂—, n = 8, R₅₁ = | H | H | H | H |
| | | CH₃) | | | | |
| (1d)-27 | Me | (P)(Q = —C(═O)—, n = 2, | H | H | H | H |
| | | R₅₁ = CH₃) | | | | |
| (1d)-28 | Me | (P)(Q = —C(═O)—, n = 3, | H | H | H | H |
| | | R₅₁ = CH₃) | | | | |
| (1d)-29 | Me | (P)(Q = —C(═O)—, n = 4, | H | H | H | H |
| | | R₅₁ = CH₃) | | | | |
| (1d)-30 | Me | (P)(Q = —C(═O)—, n = 8, | H | H | H | H |
| | | R₅₁ = CH₃) | | | | |
| (1d)-31 | n-butyl | Me | Me | H | H | H |
| (1d)-32 | n-butyl | Me | H | Me | H | H |
| (1d)-33 | n-butyl | Me | H | H | Me | H |
| (1d)-34 | n-butyl | Me | H | Ph | H | H |
| (1d)-35 | (a1) | Me | Me | H | H | H |
| (1d)-36 | (a2) | Me | H | Me | H | H |
| (1d)-37 | (a2) | Me | H | H | Me | H |
| (1d)-38 | (a3) | Me | H | Ph | H | H |
| (1d)-39 | n-butyl | Ph | H | H | H | H |
| (1d)-40 | n-butyl | Me | H | (D)-1 | H | H |
| (1d)-41 | n-butyl | H | H | H | H | H |

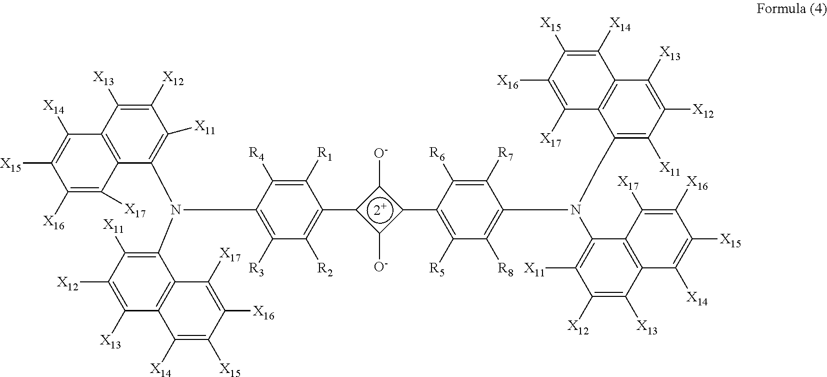

In the compound (4) having a structure represented by the above Formula (4), R₁ and R₅ are preferably an NHCORa group, an OH group, an NHSO₂Rb group, or an NHPO(ORf)(ORg) group, and each Ra is preferably an alkyl group (a). Table V illustrates such compounds (4). In the table, “tolyl” indicates a tolyl group.

TABLE V

| Com- | | | | | | | | | | | | | | | | | | | | | | | |
|---|---|---|---|---|---|---|---|---|---|---|---|---|---|---|---|---|---|---|---|---|---|---|---|
| pound | | | | | | | | | | | | | | | | | | | | | | | |
| name | | | | | | | | | | | | | | | | | | | | | | | |
| (Abbre- | | | | | | | | | | | | | | | | | | | | | | | |
| viation) | R₁ | Ra | Rb | Rf | Rg | R₂ | R₃ | R₄ | R₅ | Ra | Rb | Rf | Rg | R₆ | R₇ | R₈ | X₁₁ | X₁₂ | X₁₃ | X₁₄ | X₁₅ | X₁₈ | X₁₇ |
| (4)-1 | NHCORa | n- | — | — | — | H | H | H | NHCORa | n- | — | — | — | H | H | H | Me | H | H | H | H | H | H |
| | | butyl | | | | | | | | butyl | | | | | | | | | | | | | |
| (4)-2 | NHCORa | n- | — | — | — | H | H | H | NHCORa | n- | — | — | — | H | H | H | Me | H | Ph | H | H | H | H |
| | | butyl | | | | | | | | butyl | | | | | | | | | | | | | |
| (4)-3 | NHCORa | (a1) | — | — | — | H | H | H | NHCORa | (a1) | — | — | — | H | H | H | Me | H | H | H | H | H | H |
| (4)-4 | NHCORa | (a2) | — | — | — | H | H | H | NHCORa | (a2) | — | — | — | H | H | H | Me | H | Ph | H | H | H | H |
| (4)-5 | NHCORa | (a2) | — | — | — | H | H | H | NHCORa | (a2) | — | — | — | H | H | H | Me | H | H | H | H | H | H |
| (4)-6 | NHCORa | (a3) | — | — | — | H | H | H | NHCORa | (a3) | — | — | — | H | H | H | Me | H | Ph | H | H | H | H |
| (4)-7 | OH | — | — | — | — | H | H | H | OH | — | — | — | — | H | H | H | n-butyl | H | H | H | H | H | H |
| (4)-8 | OH | — | — | — | — | H | H | H | OH | — | — | — | — | H | H | H | (a5) | H | H | H | H | H | H |
| (4)-9 | OH | — | — | — | — | H | H | H | OH | — | — | — | — | H | H | H | (a2) | H | H | H | H | H | H |
| (4)-10 | OH | — | — | — | — | H | H | H | OH | — | — | — | — | H | H | H | (P)(n = 8, | H | H | H | H | H | H |
| | | | | | | | | | | | | | | | | | R₅₁ = | | | | | | |
| | | | | | | | | | | | | | | | | | CH₃) | | | | | | |
| (4)-11 | OH | — | — | — | — | H | H | H | OH | — | — | — | — | H | H | H | (a2) | H | Ph | H | H | H | H |
| (4)-12 | NHSO₂Rb | — | (a1) | — | — | H | H | H | NHSO₂Rb | — | (a1) | — | — | H | H | H | H | H | H | H | H | H | H |
| (4)-13 | NHSO₂Rb | — | tolyl | — | — | H | H | H | NHSO₂Rb | — | tolyl | — | — | H | H | H | (P)(n = 8, | H | H | H | H | H | H |
| | | | | | | | | | | | | | | | | | R₅₁ = | | | | | | |
| | | | | | | | | | | | | | | | | | CH₃) | | | | | | |
| (4)-14 | NH₂PO | — | — | Me | Me | H | H | H | NH₂PO | — | — | Me | Me | H | H | H | H | H | H | H | H | H | H |
| | (ORf) | | | | | | | | (ORf) | | | | | | | | | | | | | | |
| | (ORg) | | | | | | | | (ORg) | | | | | | | | | | | | | | |

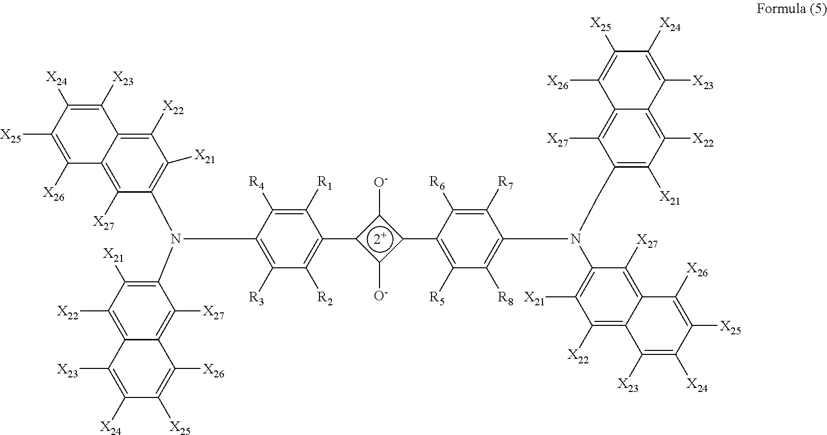

In the compound (5) having a structure represented by the above Formula (5), R₁ and R₅ arm preferably an NHCORa group, an OH group, an NHSO₂Rb group, or an NHPO(ORf)(ORg) group, and each Ra is preferably an alkyl group (a). Table VI shows such compounds (5).

TABLE VI

| Compound | | | | | | | | | | | | |
|---|---|---|---|---|---|---|---|---|---|---|---|---|
| name | | | | | | | | | | | | |
| (Abbreviation) | R₁ | Ra | Rb | Rf | Rg | R₂ | R₃ | R₄ | R₅ | Ra | Rb | Rf |
| (5)-1 | NHCORa | n- | — | — | — | H | H | H | NHCORa | n- | — | — |
| | | butyl | | | | | | | | butyl | | |
| (5)-2 | NHCORa | n- | — | — | — | H | H | H | NHCORa | n- | — | — |
| | | butyl | | | | | | | | butyl | | |
| (5)-3 | NHCORa | (a1) | — | — | — | H | H | H | NHCORa | (a1) | — | — |
| (5)-4 | NHCORa | (a2) | — | — | — | H | H | H | NHCORa | (a2) | — | — |
| (5)-5 | NHCORa | (a2) | — | — | — | H | H | H | NHCORa | (a2) | — | — |
| (5)-6 | NHCORa | (a3) | — | — | — | H | H | H | NHCORa | (a3) | — | — |
| (5)-7 | OH | — | — | — | — | H | H | H | OH | — | — | — |
| (5)-8 | OH | — | — | — | — | H | H | H | OH | — | — | — |
| (5)-9 | OH | — | — | — | — | H | H | H | OH | — | — | — |
| (5)-10 | OH | — | — | — | — | H | H | H | OH | — | — | — |
| (5)-11 | OH | — | — | — | — | H | H | H | OH | — | — | — |
| (5)-12 | OH | — | — | — | — | H | H | H | OH | — | — | — |
| (5)-13 | NHSO₂Rb | — | (a1) | — | — | H | H | H | NHSO₂Rb | — | (a1) | — |
| (5)-14 | NHSO₂Rb | — | tolyl | — | — | H | H | H | NHSO₂Rb | — | tolyl | — |
| (5)-15 | NH₂PO | — | — | Me | Me | H | H | H | NH₂PO | — | — | Me |
| | (ORf) | | | | | | | | (ORf) | | | |
| | (ORg) | | | | | | | | (ORg) | | | |
| Compound | | | | | | | | | | | | |
| name | | | | | | | | | | | | |
| (Abbreviation) | Rg | R₆ | R₇ | R₈ | X₂₁ | X₂₂ | X₂₃ | X₂₄ | X₂₅ | X₂₆ | X₂₇ | |
| (5)-1 | — | H | H | H | Me | H | H | H | H | H | H | |
| (5)-2 | — | H | H | H | Me | H | Ph | H | H | H | H | |
| (5)-3 | — | H | H | H | Me | H | H | H | H | H | H | |
| (5)-4 | — | H | H | H | Me | H | Ph | H | H | H | H | |
| (5)-5 | — | H | H | H | Me | H | H | H | H | H | H | |
| (5)-6 | — | H | H | H | Me | H | Ph | H | H | H | H | |
| (5)-7 | — | H | H | H | n- | H | H | H | H | H | H | |
| | | | | | butyl | | | | | | | |
| (5)-8 | — | H | H | H | H | H | H | H | H | H | n- | |
| | | | | | | | | | | | butyl | |
| (5)-9 | — | H | H | H | (a5) | H | H | H | H | H | H | |
| (5)-10 | — | H | H | H | (a6) | H | H | H | H | H | H | |
| (5)-11 | — | H | H | H | (P)(n = 6, | H | H | H | H | H | H | |
| | | | | | R₅₁ = CH₃) | | | | | | | |
| (5)-12 | — | H | H | H | (a6) | H | Ph | H | H | H | H | |
| (5)-13 | — | H | H | H | H | H | H | H | H | H | H | |
| (5)-14 | — | H | H | H | H | H | H | H | H | H | H | |
| (5)-15 | Me | H | H | H | (a5) | H | H | H | H | H | H | |

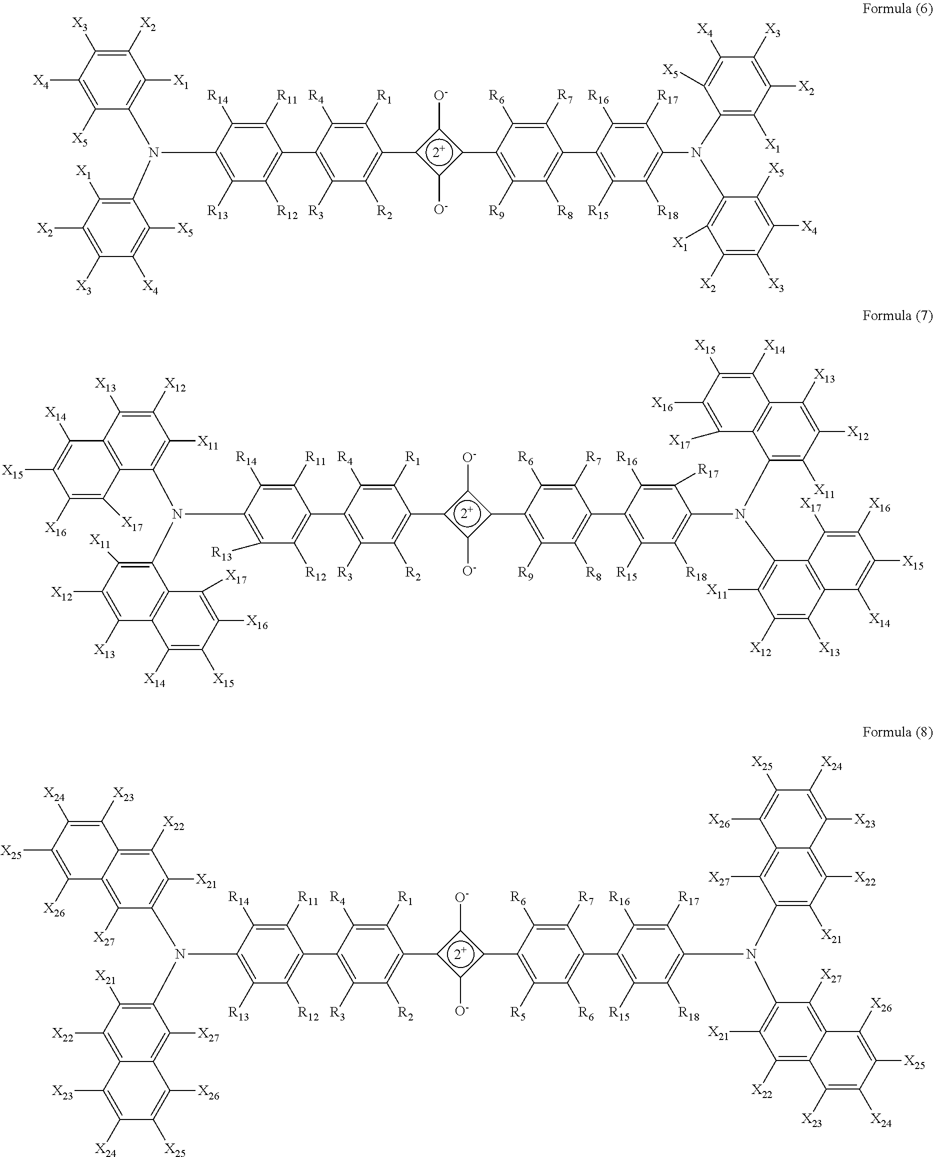

Specific examples of the respective compounds (6) to (8) having the structures represented by the above Formulas (6) to (8) are shown in Table VII, Table VIII and Table IX, respectively. In addition, in the following table, “—O-(a4)” indicates a group (a4) to which —O— is bonded as a linking group.

TABLE VII

| Compound | | | | | | | | | | | | | | | |
|---|---|---|---|---|---|---|---|---|---|---|---|---|---|---|---|
| name | | | | | | | | | | | | | | | |
| (Abbreviation) | R₁ | Ra | Rb | Rf | Rg | R₂ | R₃ | R₄ | R₅ | Ra | Rb | Rf | Rg | R₆ | R₇ |
| (6)-1 | OH | — | — | — | — | H | H | H | OH | — | — | — | — | H | H |
| (6)-2 | OH | — | — | — | — | H | H | H | OH | — | — | — | — | H | H |
| (6)-3 | OH | — | — | — | — | H | Me | H | OH | — | — | — | — | H | Me |
| (6)-4 | OH | — | — | — | — | H | H | H | OH | — | — | — | — | H | H |
| (6)-5 | NHCORa | n- | — | — | — | H | H | H | NHCORa | n- | — | — | — | H | H |
| | | butyl | | | | | | | | butyl | | | | | |
| (6)-6 | NHCORa | n- | — | — | — | H | H | H | NHCORa | n- | — | — | — | H | H |
| | | hexyl | | | | | | | | hexyl | | | | | |
| (6)-7 | NHCORa | (a1) | — | — | — | H | H | H | NHCORa | (a1) | — | — | — | H | H |
| (6)-8 | NHCORa | (a2) | — | — | — | H | H | H | NHCORa | (a2) | — | — | — | H | H |
| (6)-9 | NHCORa | (a2) | — | — | — | H | H | H | NHCORa | (a2) | — | — | — | H | H |
| (6)-10 | NHCORa | (a3) | — | — | — | H | H | H | NHCORa | (a3) | — | — | — | H | H |
| (6)-11 | NHSO₂Rb | — | (a1) | — | — | H | H | H | NHSO₂Rb | — | (a1) | — | — | H | H |
| (6)-12 | NHSO₂Rb | — | tolyl | — | — | H | H | H | NHSO₂Rb | — | tolyl | — | — | H | H |
| (6)-13 | NH₂PO | — | — | Me | Me | H | H | H | NH₂PO | — | — | Me | Me | H | H |
| | (ORf)(ORg) | | | | | | | | (ORf)(ORg) | | | | | | |
| Compound | | | | | | | | | | | | | | | |
| name | | | | | | | | | | | | | | | |
| (Abbreviation) | R₈ | R₁₁ | R₁₂ | R₁₃ | R₁₄ | R₁₅ | R₁₈ | R₁₇ | R₁₈ | X₁ | X₂ | X₃ | X₄ | X₅ | |
| (6)-1 | H | H | H | H | H | H | H | H | H | (a2) | H | H | H | H | |
| (6)-2 | H | Me | Me | H | H | Me | Me | H | H | n- | H | H | H | H | |
| | | | | | | | | | | butyl | | | | | |
| (6)-3 | H | H | H | H | H | H | H | H | H | *1 | H | H | H | H | |
| (6)-4 | H | H | H | H | H | H | H | H | H | n- | H | Ph | H | H | |
| | | | | | | | | | | octyl | | | | | |
| (6)-5 | H | H | H | H | H | H | H | H | H | Me | H | H | H | H | |
| (6)-6 | H | H | H | H | H | H | H | H | H | Me | H | Ph | H | H | |
| (6)-7 | H | H | H | H | H | H | H | H | H | Me | H | H | H | H | |
| (6)-8 | H | H | H | H | H | H | H | H | H | Me | H | H | H | H | |
| (6)-9 | H | H | H | H | H | H | H | H | H | Me | H | H | Me | H | |
| (6)-10 | H | H | H | H | H | H | H | H | H | Me | H | Ph | H | H | |
| (6)-11 | H | H | H | H | H | H | H | H | H | Me | H | H | H | H | |
| (6)-12 | H | H | H | H | H | H | H | H | H | Me | H | H | H | H | |
| (6)-13 | H | H | H | H | H | H | H | H | H | Me | H | H | H | H | |

*1: (P)(Q = —CH₂—, n = 9, R₅₁ = H)

TABLE VIII

| Compound | | | | | | | | | | | | | | | | |
|---|---|---|---|---|---|---|---|---|---|---|---|---|---|---|---|---|
| name | | | | | | | | | | | | | | | | |
| (Abbreviation) | R₁ | Ra | Rb | Rf | Rg | R₂ | R₃ | R₄ | R₅ | Ra | Rb | Rf | Rg | R₆ | R₇ | R₈ |
| (7)-1 | NHCORa | n- | — | — | — | H | H | H | NHCORa | n- | — | — | — | H | H | H |
| | | butyl | | | | | | | | butyl | | | | | | |
| (7)-2 | NHCORa | n- | — | — | — | H | H | H | NHCORa | n- | — | — | — | H | H | H |
| | | hexyl | | | | | | | | hexyl | | | | | | |
| (7)-3 | NHCORa | (a1) | — | — | — | H | H | H | NHCORa | (a1) | — | — | — | H | H | H |
| (7)-4 | NHCORa | (a2) | — | — | — | H | H | H | NHCORa | (a2) | — | — | — | H | H | H |
| (7)-5 | NHCORa | (a2) | — | — | — | H | H | H | NHCORa | (a2) | — | — | — | H | H | H |
| (7)-6 | NHCORa | (a3) | — | — | — | H | H | H | NHCORa | (a3) | — | — | — | H | H | H |
| (7)-7 | OH | — | — | — | — | H | H | H | OH | — | — | — | — | H | H | H |
| (7)-8 | OH | — | — | — | — | H | H | H | OH | — | — | — | — | H | H | H |
| (7)-9 | OH | — | — | — | — | H | Me | H | OH | — | — | — | — | H | Me | H |
| (7)-10 | OH | — | — | — | — | H | H | H | OH | — | — | — | — | H | H | H |
| (7)-11 | NHSO₂Rb | — | (a1) | — | — | H | H | H | NHSO₂Rb | — | (a1) | — | — | H | H | H |
| (7)-12 | NHSO₂Rb | — | tolyl | — | — | H | H | H | NHSO₂Rb | — | tolyl | — | — | H | H | H |
| (7)-13 | *2 | — | — | Me | Me | H | H | H | *2 | — | — | Me | Me | H | H | H |
| Compound | | | | | | | | | | | | | | | | |
| name | | | | | | | | | | | | | | | | |
| (Abbreviation) | R₁₁ | R₁₂ | R₁₃ | R₁₄ | R₁₅ | R₁₈ | R₁₇ | R₁₈ | X₁₁ | X₁₂ | X₁₃ | X₁₄ | X₁₅ | X₁₆ | X₁₇ | |
| (7)-1 | H | H | H | H | H | H | H | H | Me | H | H | H | H | H | H | |
| (7)-2 | H | H | H | H | H | H | H | H | Me | H | Ph | H | H | H | H | |
| (7)-3 | H | H | H | H | H | H | H | H | Me | H | H | H | H | H | H | |
| (7)-4 | H | H | H | H | H | H | H | H | H | H | H | H | H | H | H | |
| (7)-5 | H | H | H | H | H | H | H | H | Me | H | Me | H | H | H | H | |
| (7)-6 | H | H | H | H | H | H | H | H | Me | H | Ph | H | H | H | H | |
| (7)-7 | H | H | H | H | H | H | H | H | (a2) | H | H | H | H | H | H | |
| (7)-8 | Me | Me | H | H | Me | Me | H | H | n- | H | H | H | H | H | H | |
| | | | | | | | | | octyl | | | | | | | |
| (7)-9 | Me | Me | H | H | Me | Me | H | H | (a5) | H | H | H | H | H | H | |
| (7)-10 | H | H | H | H | H | H | H | H | *1 | H | Ph | H | H | H | H | |
| (7)-11 | H | H | H | H | H | H | H | H | H | H | H | H | H | H | H | |
| (7)-12 | H | H | H | H | H | H | H | H | H | H | H | H | H | H | H | |
| (7)-13 | H | H | H | H | H | H | H | H | *1 | H | H | H | H | H | H | |

*1: (P)(n = 8, R₅₁ = CH₃)
*2: NH₂PO(ORf)(ORg)

TABLE IX

| Compound | | | | | | | | | | | | | | | | |
|---|---|---|---|---|---|---|---|---|---|---|---|---|---|---|---|---|
| name | | | | | | | | | | | | | | | | |
| (Abbreviation) | R₁ | Ra | Rb | Rf | Rg | R₂ | R₃ | R₄ | R₅ | Ra | Rb | Rf | Rg | R₆ | R₇ | R₈ |
| (8)-1 | NHCORa | n- | — | — | — | H | H | H | NHCORa | n- | — | — | — | H | H | H |
| | | butyl | | | | | | | | butyl | | | | | | |
| (8)-2 | NHCORa | (a1) | — | — | — | H | H | H | NHCORa | (a1) | — | — | — | H | H | H |
| (8)-3 | NHCORa | (a2) | — | — | — | H | H | H | NHCORa | (a2) | — | — | — | H | H | H |
| (8)-4 | NHCORa | (a3) | — | — | — | H | H | H | NHCORa | (a3) | — | — | — | H | H | H |
| (8)-5 | OH | — | — | — | — | H | H | H | OH | — | — | — | — | H | H | H |
| (8)-6 | OH | — | — | — | — | H | H | H | OH | — | — | — | — | H | H | H |
| (8)-7 | OH | — | — | — | — | H | H | H | OH | — | — | — | — | H | H | H |
| (8)-8 | OH | — | — | — | — | H | H | H | OH | — | — | — | — | H | H | H |
| (8)-9 | OH | — | — | — | — | H | H | H | OH | — | — | — | — | H | H | H |
| (8)-10 | OH | — | — | — | — | H | H | H | OH | — | — | — | — | H | H | H |
| (8)-11 | NHSO₂Rb | — | (a1) | — | — | H | H | H | NHSO₂Rb | — | (a1) | — | — | H | H | H |
| (8)-12 | NHSO₂Rb | — | tolyl | — | — | H | H | H | NHSO₂Rb | — | tolyl | — | — | H | H | H |
| (8)-13 | *2 | — | — | Me | Me | H | H | H | *2 | — | — | Me | Me | H | H | H |
| Compound | | | | | | | | | | | | | | | | |
| name | | | | | | | | | | | | | | | | |
| (Abbreviation) | R₁₁ | R₁₂ | R₁₃ | R₁₄ | R₁₅ | R₁₆ | R₁₇ | R₁₈ | X₂₁ | X₂₂ | X₂₃ | X₂₄ | X₂₅ | X₂₆ | X₂₇ | |
| (8)-1 | H | H | H | H | H | H | H | H | Me | H | H | H | H | H | H | |
| (8)-2 | H | H | H | H | H | H | H | H | Me | H | H | H | H | H | H | |
| (8)-3 | H | H | H | H | H | H | H | H | H | H | H | H | H | H | H | |
| (8)-4 | H | H | H | H | H | H | H | H | Me | H | H | H | H | H | H | |
| (8)-5 | H | H | H | H | H | H | H | H | n- | H | H | H | H | H | H | |
| | | | | | | | | | butyl | | | | | | | |
| (8)-6 | Me | Me | H | H | Me | Me | H | H | H | H | H | H | H | H | H | |
| (8)-7 | Me | Me | H | H | Me | Me | H | H | (a5) | H | H | H | H | H | H | |
| (8)-8 | H | H | H | H | H | H | H | H | (a5) | H | H | H | H | H | H | |
| (8)-9 | H | H | H | H | H | H | H | H | *1 | H | H | H | H | H | H | |
| (8)-10 | —O—(a4) | H | H | H | —O—(a4) | H | H | H | (a4) | H | H | H | H | H | H | |
| (8)-11 | H | H | H | H | H | H | H | H | H | H | H | H | H | H | H | |
| (8)-12 | H | H | H | H | H | H | H | H | H | H | H | H | H | H | H | |
| (8)-13 | (a6) | H | H | H | (a6) | H | H | H | H | H | H | H | H | H | H | |

*1: (P)(n = 9, R₅₁ = CH₃)
*2: NH₂PO(ORf)(ORg)

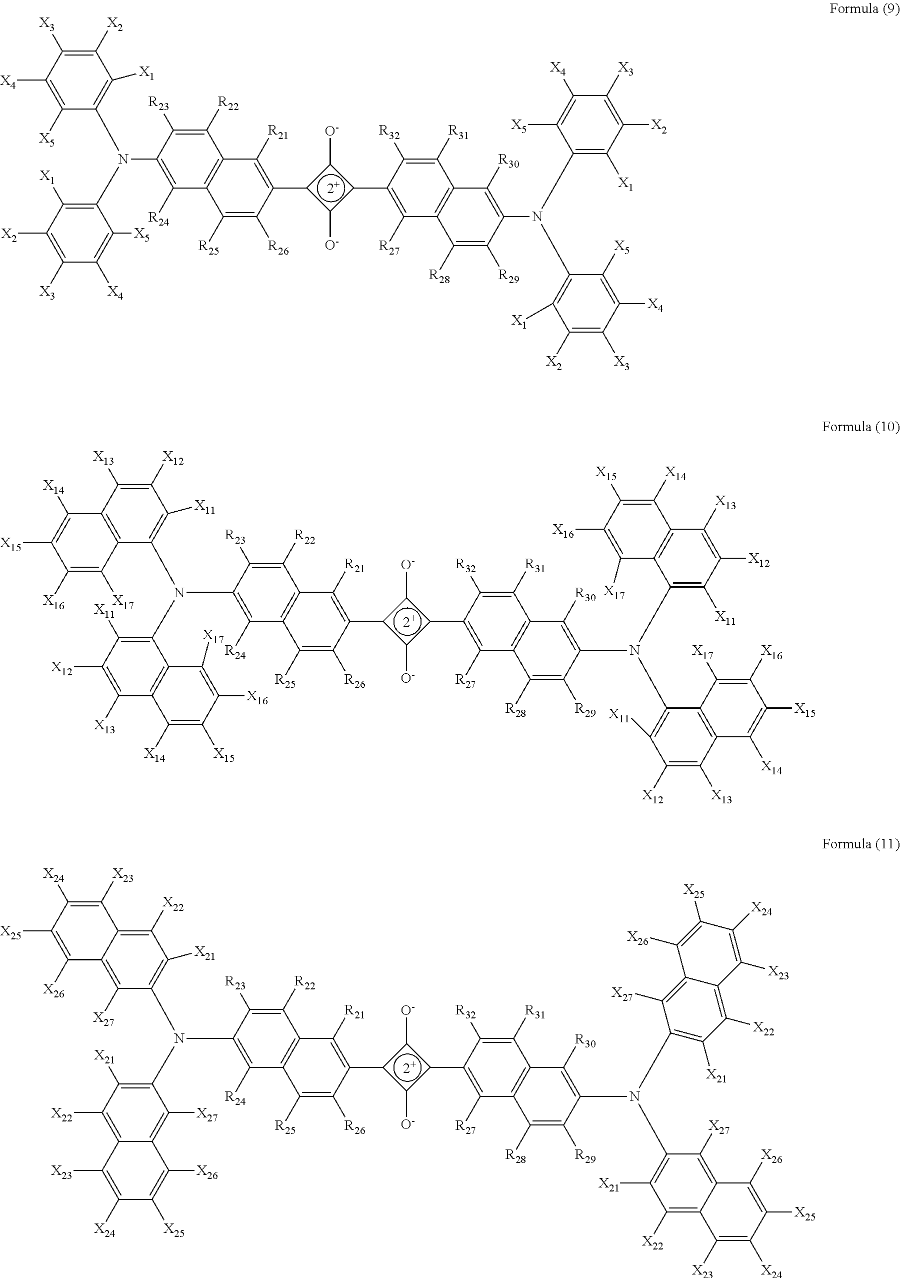

Specific examples of each of the compounds (9) to (11) having the structures represented by the above Formulas (9) to (11) are shown in Table X, Table XI and Table XII, respectively.

TABLE X

| Compound | | | | | | | | | | | | | |
|---|---|---|---|---|---|---|---|---|---|---|---|---|---|
| name | | | | | | | | | | | | | |
| (Abbreviation) | R₂₁ | Ra | Rb | Rf | Rg | R₂₂ | R₂₃ | R₂₄ | R₂₅ | R₂₈ | R₂₇ | Ra | Rb |
| (9)-1 | OH | — | — | — | — | H | H | H | H | H | OH | — | — |
| (9)-2 | OH | — | — | — | — | H | H | H | H | H | OH | — | — |
| (9)-3 | OH | — | — | — | — | H | H | H | H | H | OH | — | — |
| (9)-4 | OH | — | — | — | — | H | H | H | H | H | OH | — | — |
| (9)-5 | NHCORa | n-butyl | — | — | — | H | H | H | H | H | NHCORa | n-butyl | — |
| (9)-6 | NHCORa | n-hexyl | — | — | — | H | H | H | H | H | NHCORa | n-hexyl | — |
| (9)-7 | NHCORa | (a1) | — | — | — | H | H | H | H | H | NHCORa | (a1) | — |
| (9)-8 | NHCORa | (a2) | — | — | — | H | H | H | H | H | NHCORa | (a2) | — |
| (9)-9 | NHCORa | (a2) | — | — | — | H | H | H | H | H | NHCORa | (a2) | — |
| (9)-10 | NHCORa | (a3) | — | — | — | H | H | H | H | H | NHCORa | (a3) | — |
| (9)-11 | NHSO₂Rb | — | (a1) | — | — | H | H | H | H | H | NHSO₂Rb | — | (a1) |
| (9)-12 | NHSO₂Rb | — | tolyl | — | — | H | H | H | H | H | NHSO₂Rb | — | tolyl |
| (9)-13 | NH₂PO | — | — | Me | Me | H | H | H | H | H | NH₂PO | — | — |
| | (ORf)(ORg) | | | | | | | | | | (ORf)(ORg) | | |
| Compound | | | | | | | | | | | | | |
| name | | | | | | | | | | | | | |
| (Abbreviation) | Rf | Rg | R₂₈ | R₂₉ | R₃₀ | R₃₁ | R₃₂ | X₁ | X₂ | X₃ | X₄ | X₅ | |
| (9)-1 | — | — | H | H | H | H | H | (a6) | H | H | H | H | |
| (9)-2 | — | — | H | H | H | H | H | n-butyl | H | Me | H | H | |
| (9)-3 | — | — | H | H | H | H | H | (P)(n = 9, | H | H | H | H | |
| | | | | | | | | R₅₁ = CH₃) | | | | | |
| (9)-4 | — | — | H | H | H | H | H | n-octyl | H | Ph | H | H | |
| (9)-5 | — | — | H | H | H | H | H | Me | H | H | H | H | |
| (9)-6 | — | — | H | H | H | H | H | Me | H | Ph | H | H | |
| (9)-7 | — | — | H | H | H | H | H | Me | H | H | H | H | |
| (9)-8 | — | — | H | H | H | H | H | Me | H | H | H | H | |
| (9)-9 | — | — | H | H | H | H | H | Me | H | H | Me | H | |
| (9)-10 | — | — | H | H | H | H | H | Me | H | Ph | H | H | |
| (9)-11 | — | — | H | H | H | H | H | H | H | H | H | H | |
| (9)-12 | — | — | H | H | H | H | H | H | H | H | H | H | |
| (9)-13 | Me | Me | H | H | H | H | H | (a6) | H | H | H | H | |

TABLE XI

| Compound | | | | | | | | | | | | | | |
|---|---|---|---|---|---|---|---|---|---|---|---|---|---|---|
| name | | | | | | | | | | | | | | |
| (Abbreviation) | R₂₁ | Ra | Rb | Rf | Rg | R₂₂ | R₂₃ | R₂₄ | R₂₅ | R₂₆ | R₂₇ | Ra | Rb | Rf |
| (10)-1 | NHCORa | n- | — | — | — | H | H | H | H | H | NHCORa | n- | — | — |
| | | butyl | | | | | | | | | | butyl | | |
| (10)-2 | NHCORa | n- | — | — | — | H | H | H | H | H | NHCORa | n- | — | — |
| | | hexyl | | | | | | | | | | hexyl | | |
| (10)-3 | NHCORa | (a1) | — | — | — | H | H | H | H | H | NHCORa | (a1) | — | — |
| (10)-4 | NHCORa | (a2) | — | — | — | H | H | H | H | H | NHCORa | (a2) | — | — |
| (10)-5 | NHCORa | (a2) | — | — | — | H | H | H | H | H | NHCORa | (a2) | — | — |
| (10)-6 | NHCORa | (a3) | — | — | — | H | H | H | H | H | NHCORa | (a3) | — | — |
| (10)-7 | OH | — | — | — | — | H | H | H | H | H | OH | — | — | — |
| (10)-8 | OH | — | — | — | — | H | H | H | H | H | OH | — | — | — |
| (10)-9 | OH | — | — | — | — | H | H | H | H | H | OH | — | — | — |
| (10)-10 | OH | — | — | — | — | H | H | H | H | H | OH | — | — | — |
| (10)-11 | NHSO₂Rb | — | (a1) | — | — | H | H | H | H | H | NHSO₂Rb | — | (a1) | — |
| (10)-12 | NHSO₂Rb | — | tolyl | — | — | H | H | H | H | H | NHSO₂Rb | — | tolyl | — |
| (10)-13 | *2 | — | Me | Me | H | H | H | H | H | H | *2 | — | — | Me |
| Compound | | | | | | | | | | | | | | |
| name | | | | | | | | | | | | | | |
| (Abbreviation) | Rg | R₂₈ | R₂₉ | R₃₀ | R₃₁ | R₃₂ | X₁₁ | X₁₂ | X₁₃ | X₁₄ | X₁₅ | X₁₆ | X₁₇ | |
| (10)-1 | — | H | H | H | H | H | Me | H | H | H | H | H | H | |
| (10)-2 | — | H | H | H | H | H | H | H | H | H | H | H | H | |
| (10)-3 | — | H | H | H | H | H | Me | H | H | H | H | H | H | |
| (10)-4 | — | H | H | H | H | H | Me | H | H | H | H | H | H | |
| (10)-5 | — | H | H | H | H | H | H | H | Me | H | H | H | H | |
| (10)-6 | — | H | H | H | H | H | Me | H | Ph | H | H | H | H | |
| (10)-7 | — | H | H | H | H | H | (a7) | H | H | H | H | H | H | |
| (10)-8 | — | H | H | H | H | H | (a6) | H | H | H | H | H | H | |
| (10)-9 | — | H | H | H | H | H | *1 | H | H | H | H | H | H | |
| (10)-10 | — | H | H | H | H | H | n- | H | Ph | H | H | H | H | |
| | | | | | | | octyl | | | | | | | |
| (10)-11 | — | H | H | H | H | H | H | H | H | H | H | H | H | |
| (10)-12 | — | H | H | H | H | H | H | H | H | H | H | H | H | |
| (10)-13 | Me | H | H | H | H | H | n-octyl | H | H | H | H | H | H | |

*1: (P)(n = 9, R₅₁ = CH₃)
*2: NH₂PO(ORf)(ORg)

TABLE XII

| Compound | | | | | | | | | | | | | | |
|---|---|---|---|---|---|---|---|---|---|---|---|---|---|---|
| name | | | | | | | | | | | | | | |
| (Abbreviation) | R₂₁ | Ra | Rb | Rf | Rg | R₂₂ | R₂₃ | R₂₄ | R₂₅ | R₂₆ | R₂₇ | Ra | Rb | Rf |
| (11)-1 | NHCORa | n-butyl | — | — | — | H | H | H | H | H | NHCORa | n-butyl | — | — |
| (11)-2 | NHCORa | (a1) | — | — | — | H | H | H | H | H | NHCORa | (a1) | — | — |
| (11)-3 | NHCORa | (a2) | — | — | — | H | H | H | H | H | NHCORa | (a2) | — | — |
| (11)-4 | OH | — | — | — | — | H | H | H | H | H | OH | — | — | — |
| (11)-5 | OH | — | — | — | — | H | H | H | H | H | OH | — | — | — |
| (11)-6 | OH | — | — | — | — | H | H | H | H | H | OH | — | — | — |
| (11)-7 | OH | — | — | — | — | H | H | H | H | H | OH | — | — | — |
| (11)-8 | NHSO₂Rb | — | (a1) | — | — | H | H | H | H | H | NHSO₂Rb | — | (a1) | — |
| (11)-9 | NHSO₂Rb | — | tolyl | — | — | H | H | H | H | H | NHSO₂Rb | — | tolyl | — |
| (11)-10 | *1 | — | — | Me | Me | H | H | H | H | H | *1 | — | — | Me |
| Compound | | | | | | | | | | | | | | |
| name | | | | | | | | | | | | | | |
| (Abbreviation) | Rg | R₂₈ | R₂₉ | R₃₀ | R₃₁ | R₃₂ | X₂₁ | X₂₂ | X₂₃ | X₂₄ | X₂₅ | X₂₆ | X₂₇ | |
| (11)-1 | — | H | H | H | H | H | Me | H | H | H | H | H | H | |
| (11)-2 | — | H | H | H | H | H | H | H | H | H | H | H | H | |
| (11)-3 | — | H | H | H | H | H | H | H | H | H | H | H | H | |
| (11)-4 | — | H | H | H | H | H | (a7) | H | H | H | H | H | H | |
| (11)-5 | — | H | H | H | H | H | n-butyl | H | Me | H | H | H | H | |
| (11)-6 | — | H | H | H | H | H | (P)(n = 8, R₅₁ = | H | H | H | H | H | H | |
| | | | | | | | CH₃) | | | | | | | |
| (11)-7 | — | H | H | H | H | H | n-octyl | H | H | H | H | H | H | |
| (11)-8 | — | H | H | H | H | H | H | H | H | H | H | H | H | |
| (11)-9 | — | H | H | H | H | H | H | H | H | H | H | H | H | |
| (11)-10 | Me | H | H | H | H | H | (P)(n = 9, R₅₁ = | H | H | H | H | H | H | |
| | | | | | | | CH₃) | | | | | | | |

*1: NH₂PO(ORf)(ORg)

Further, as the compounds used in the dye according to the present invention, the following compounds may also be used.

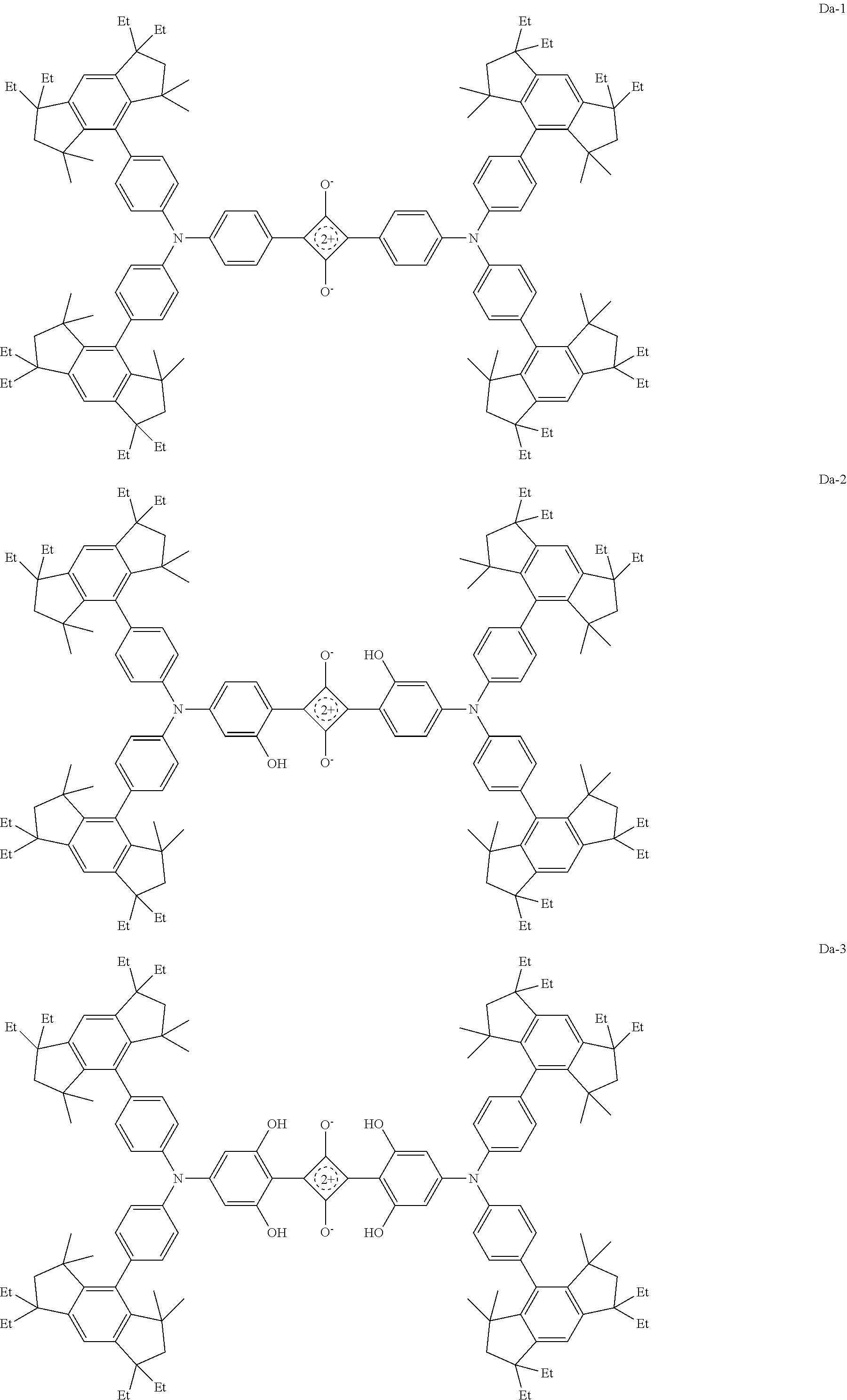

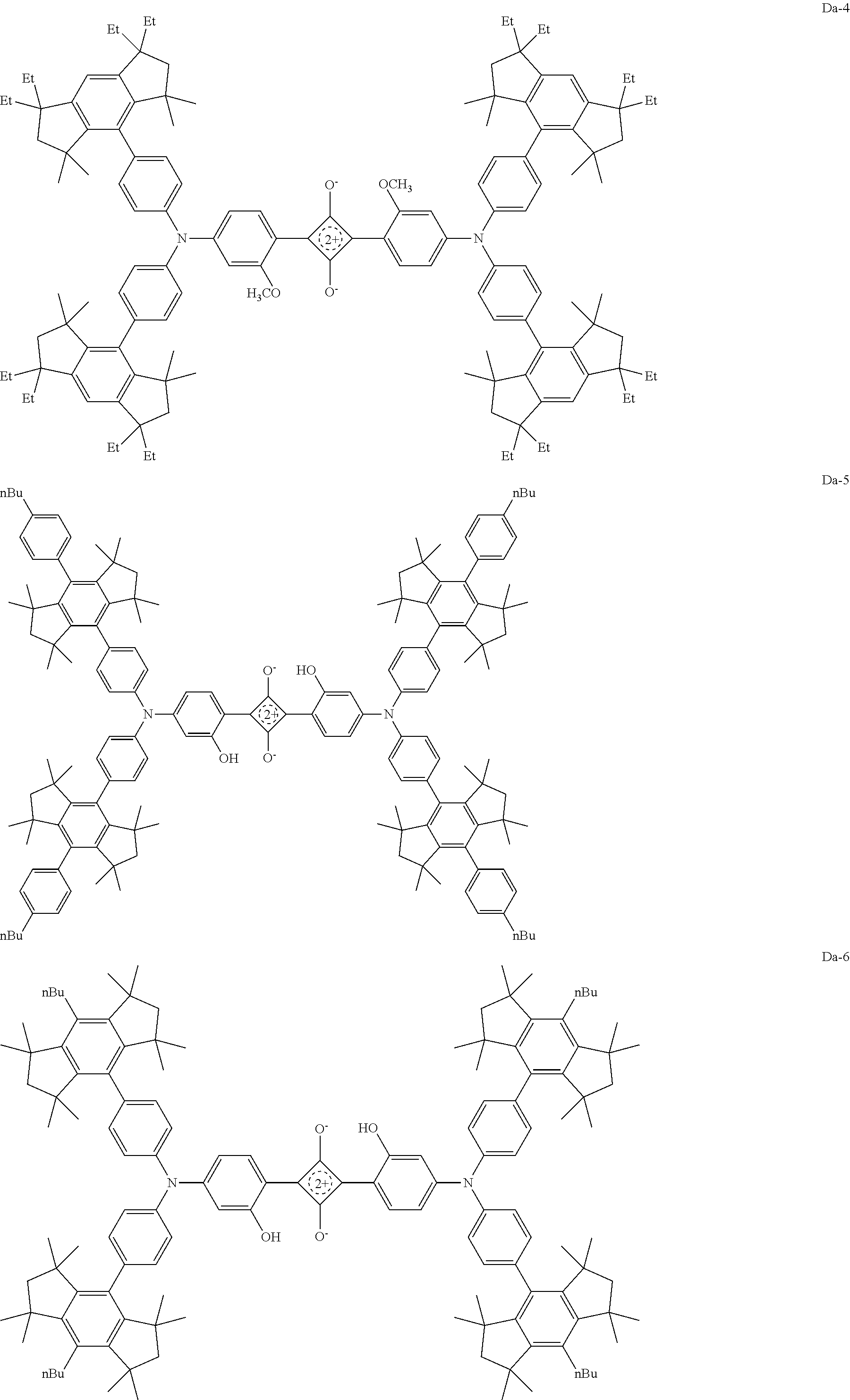

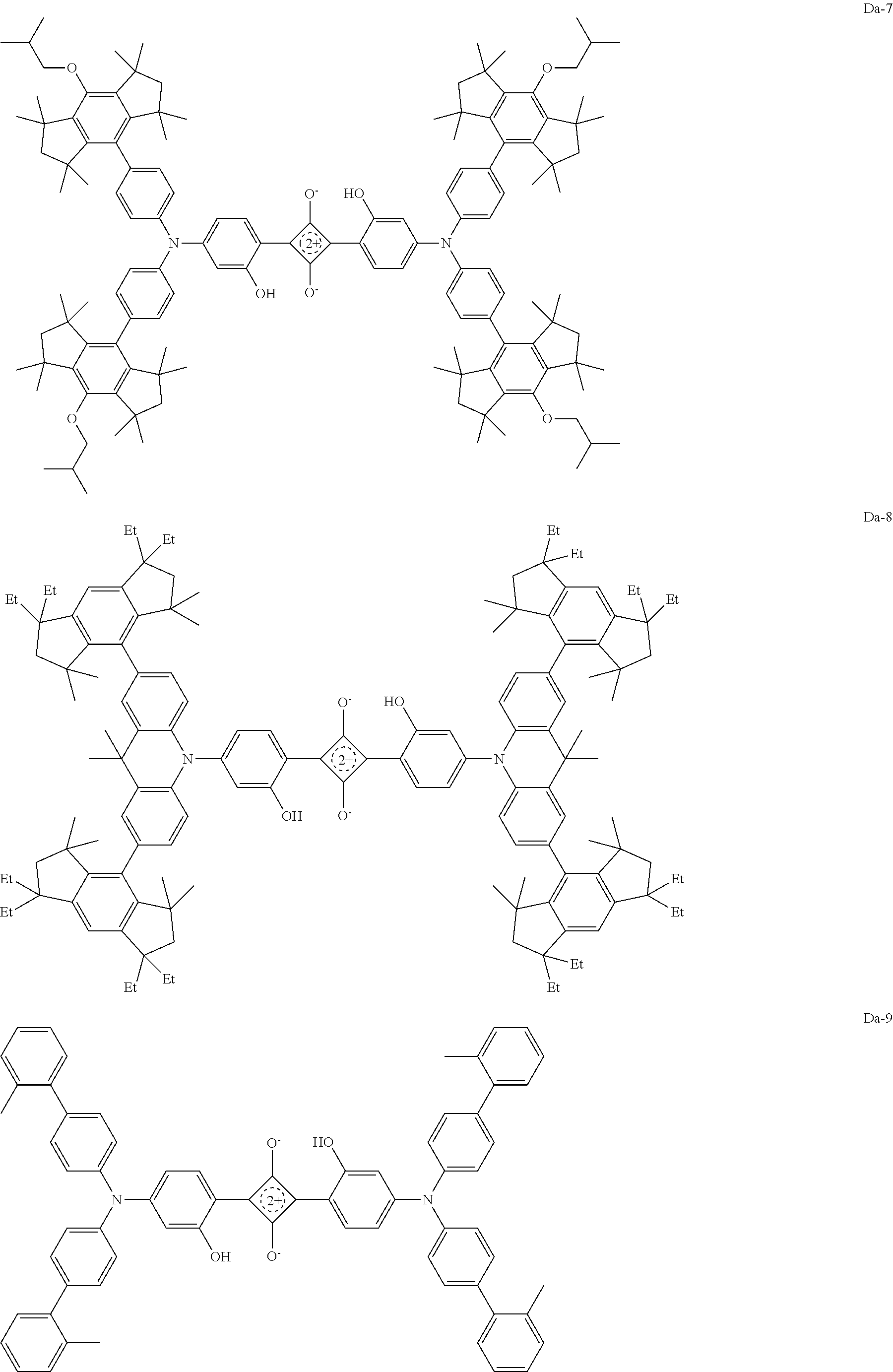

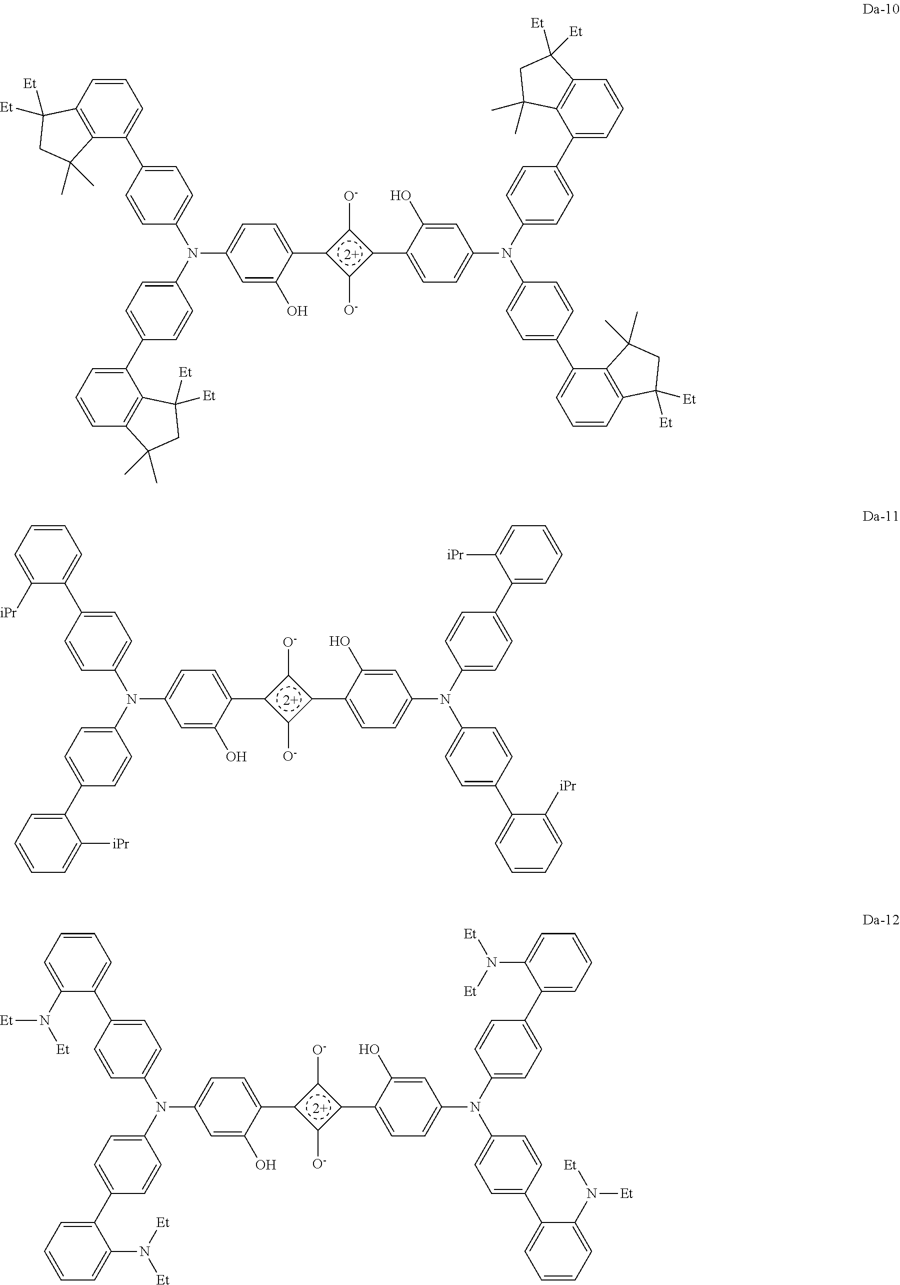

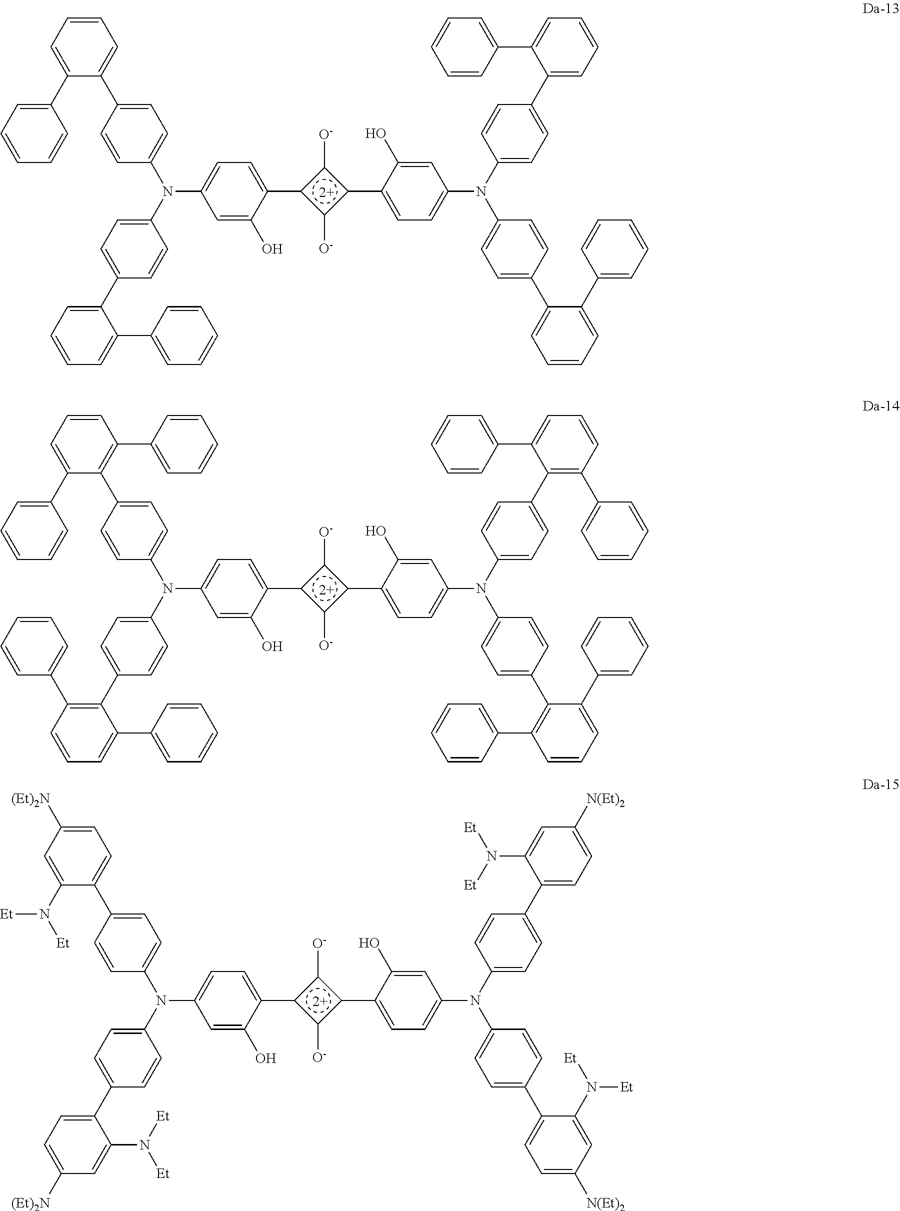

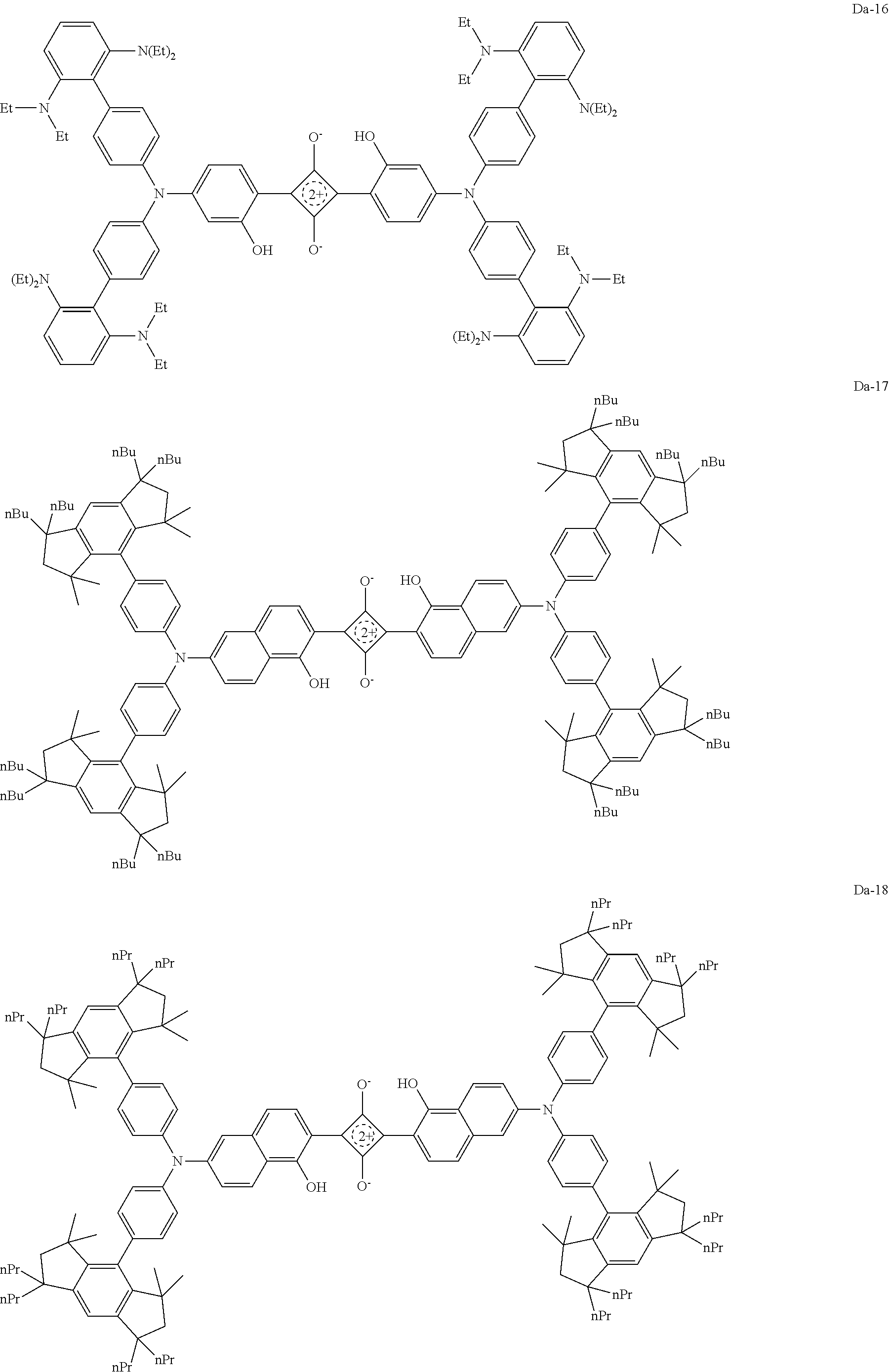

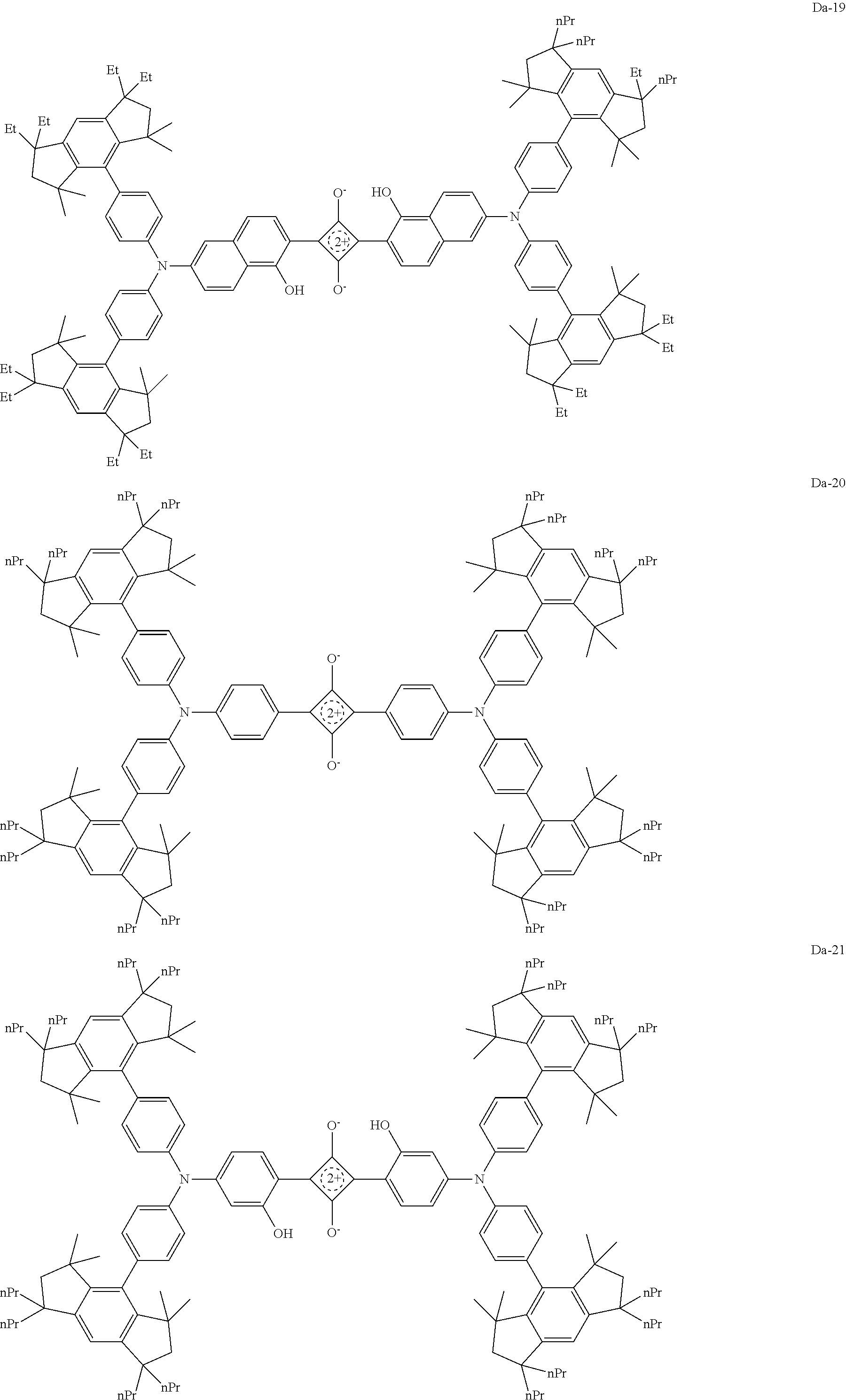

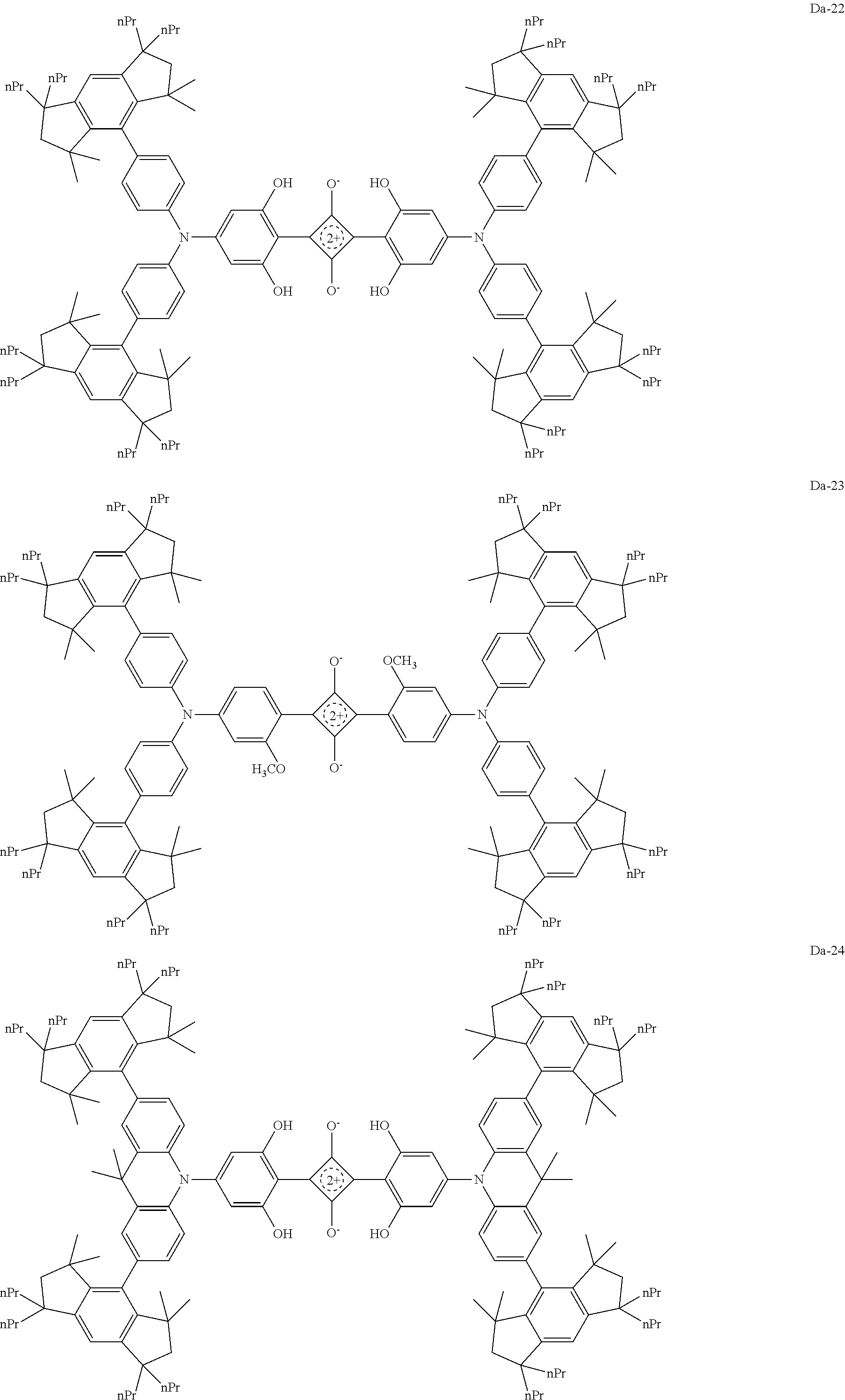

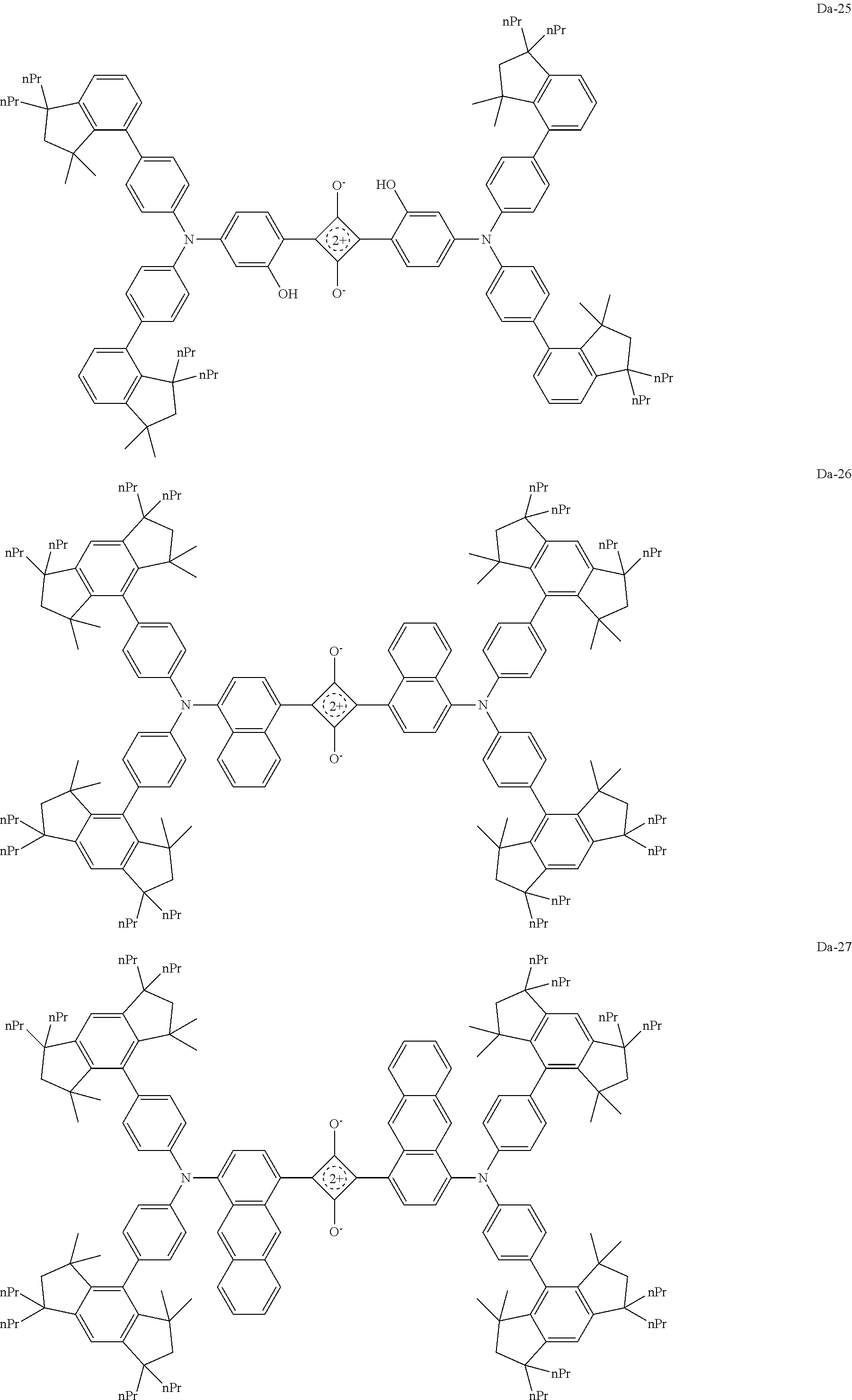

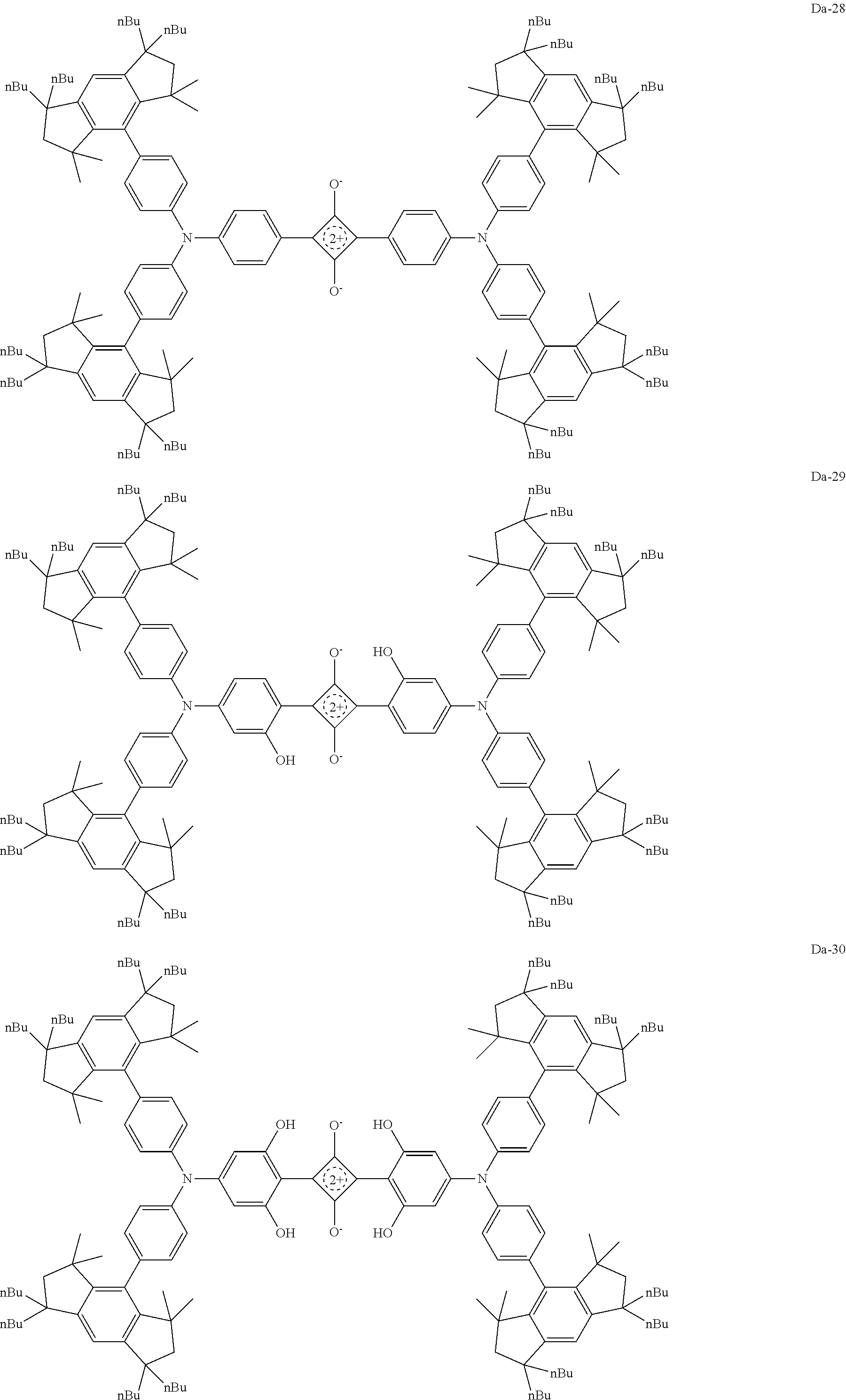

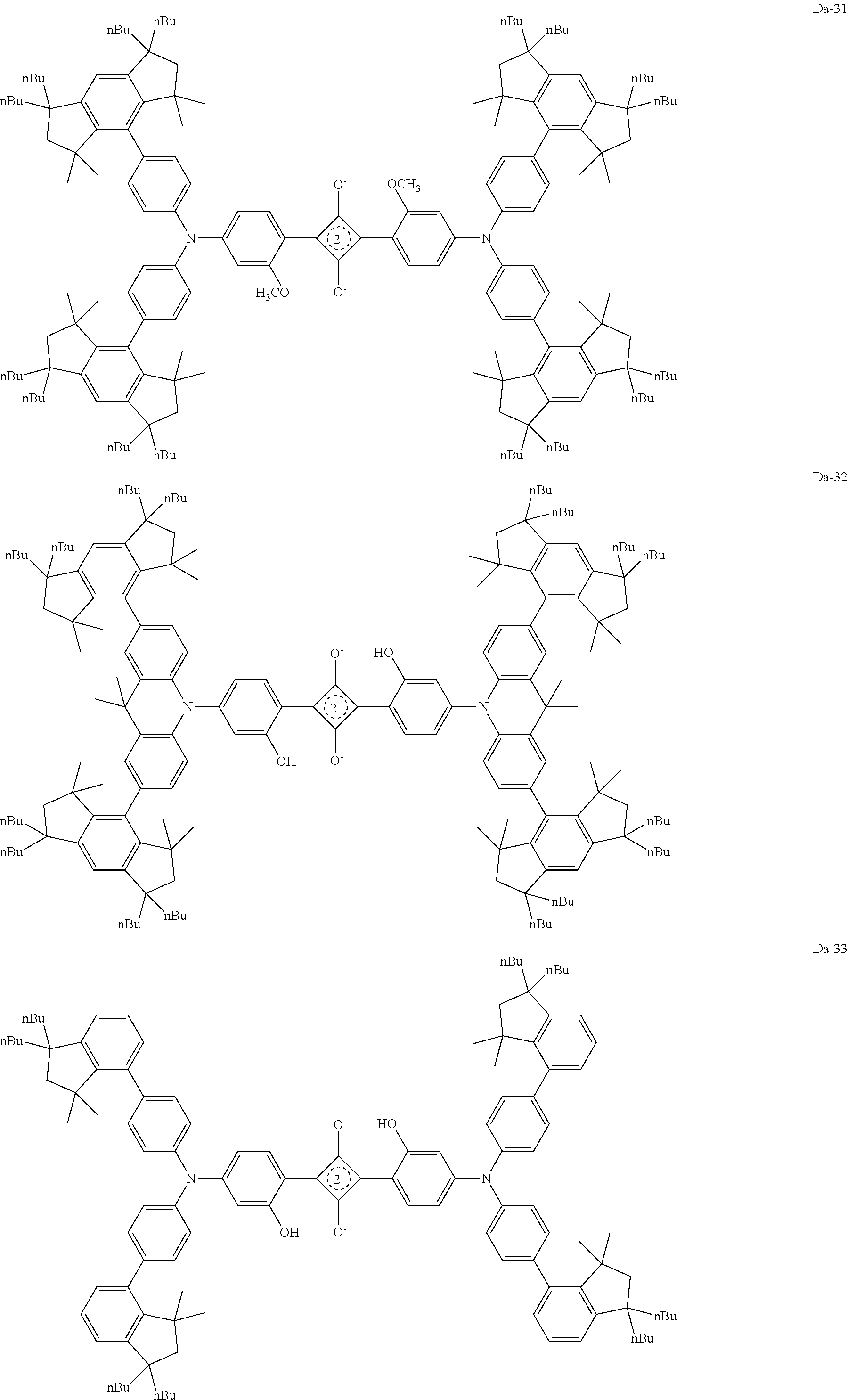

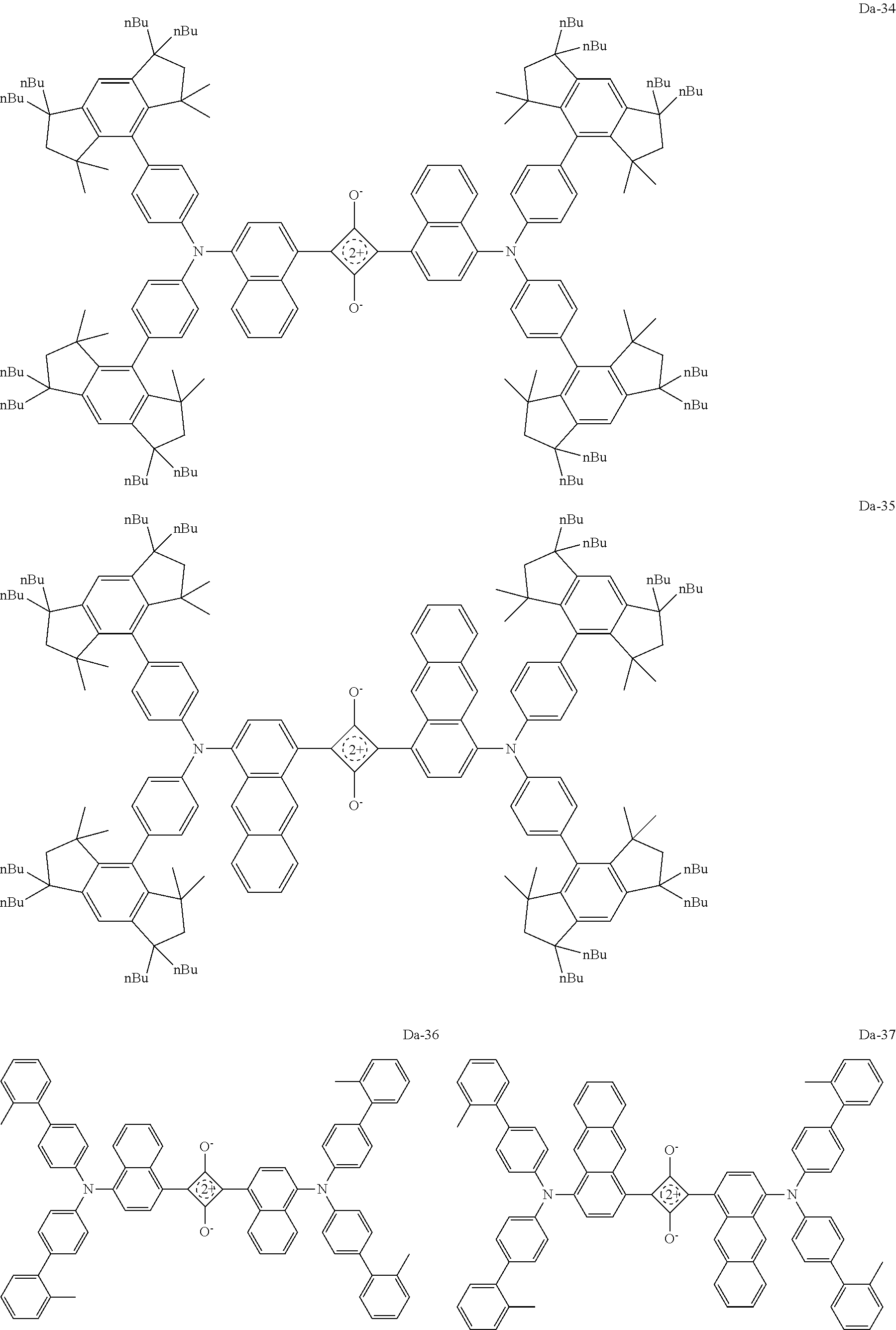

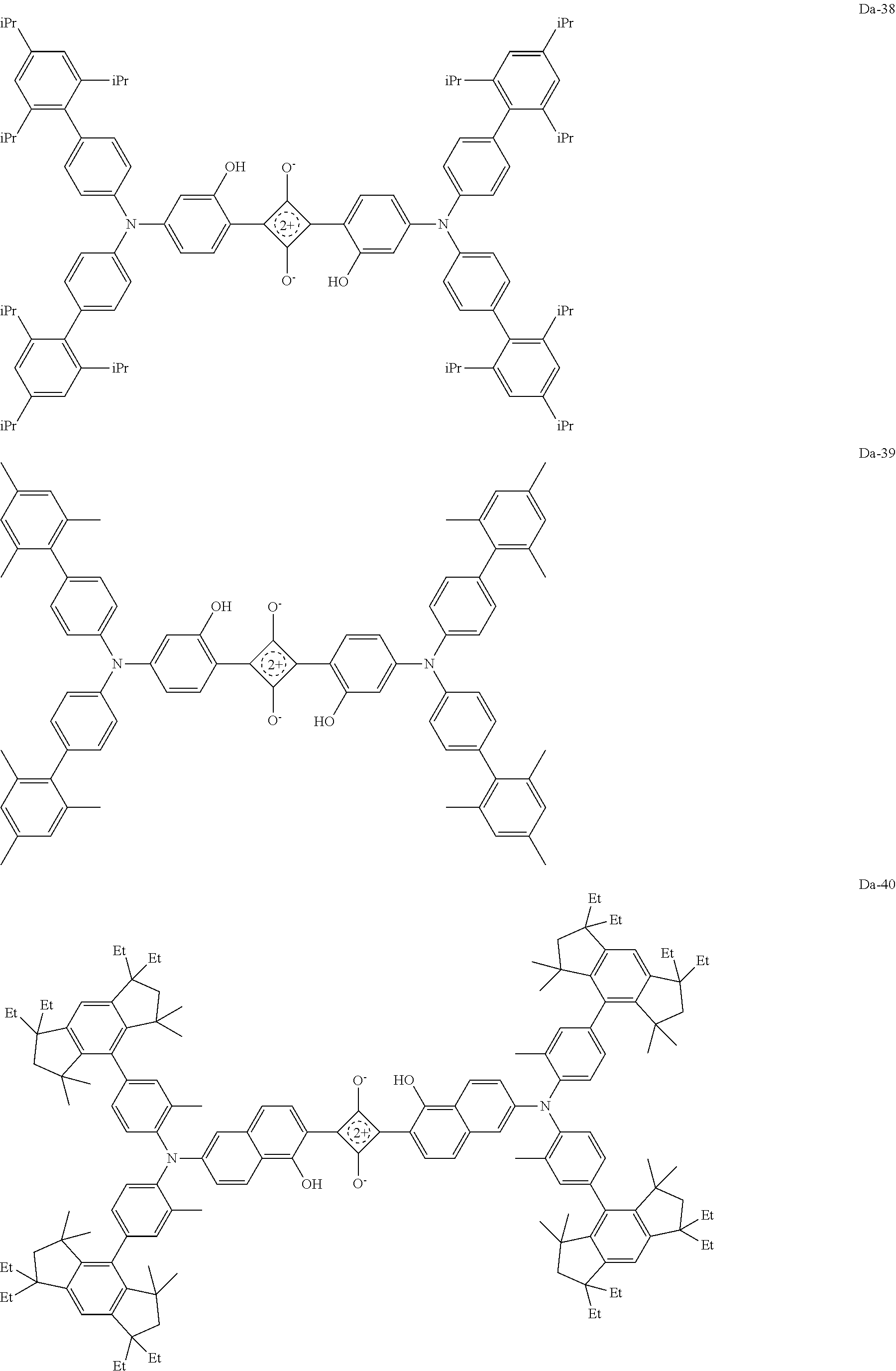

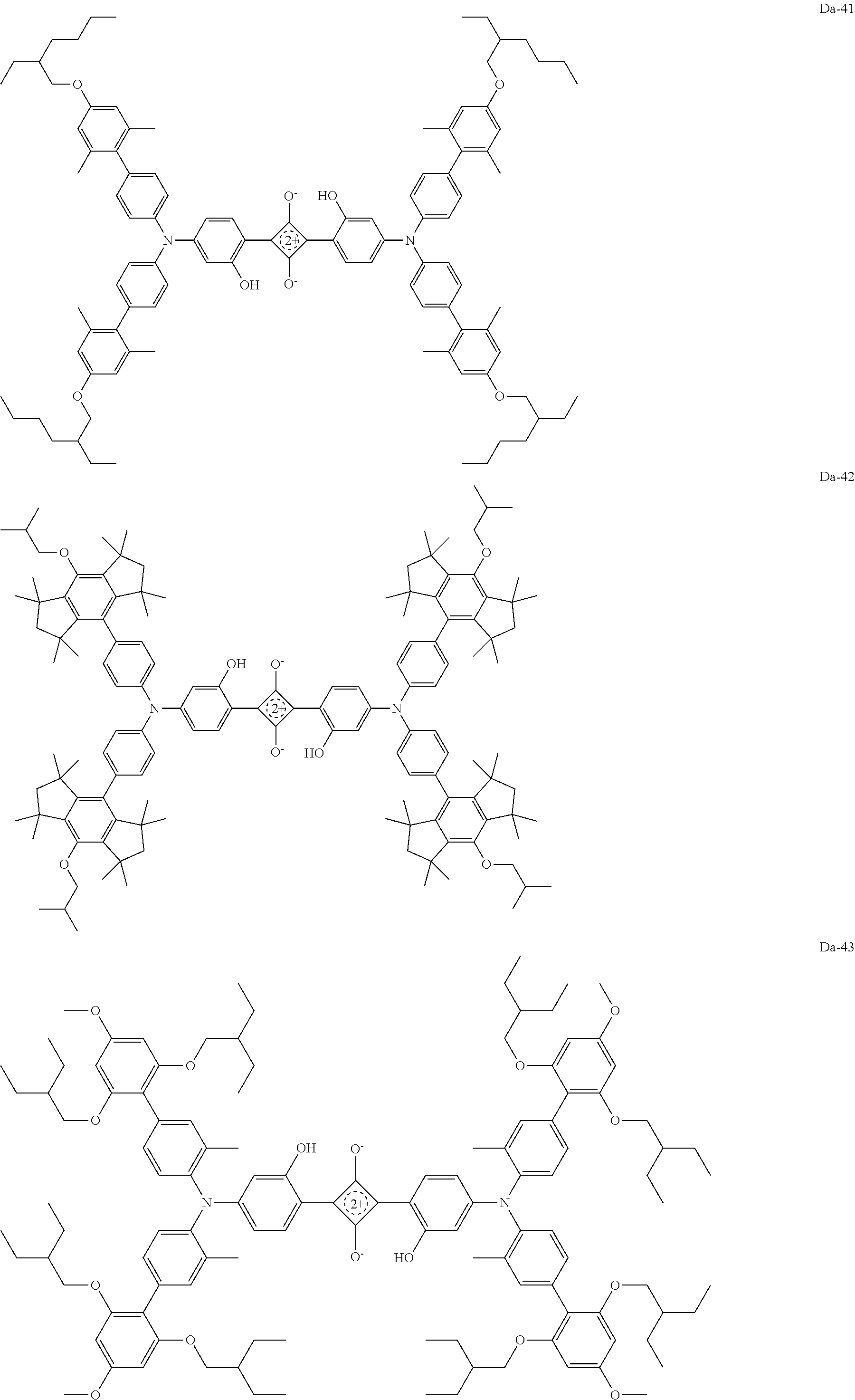

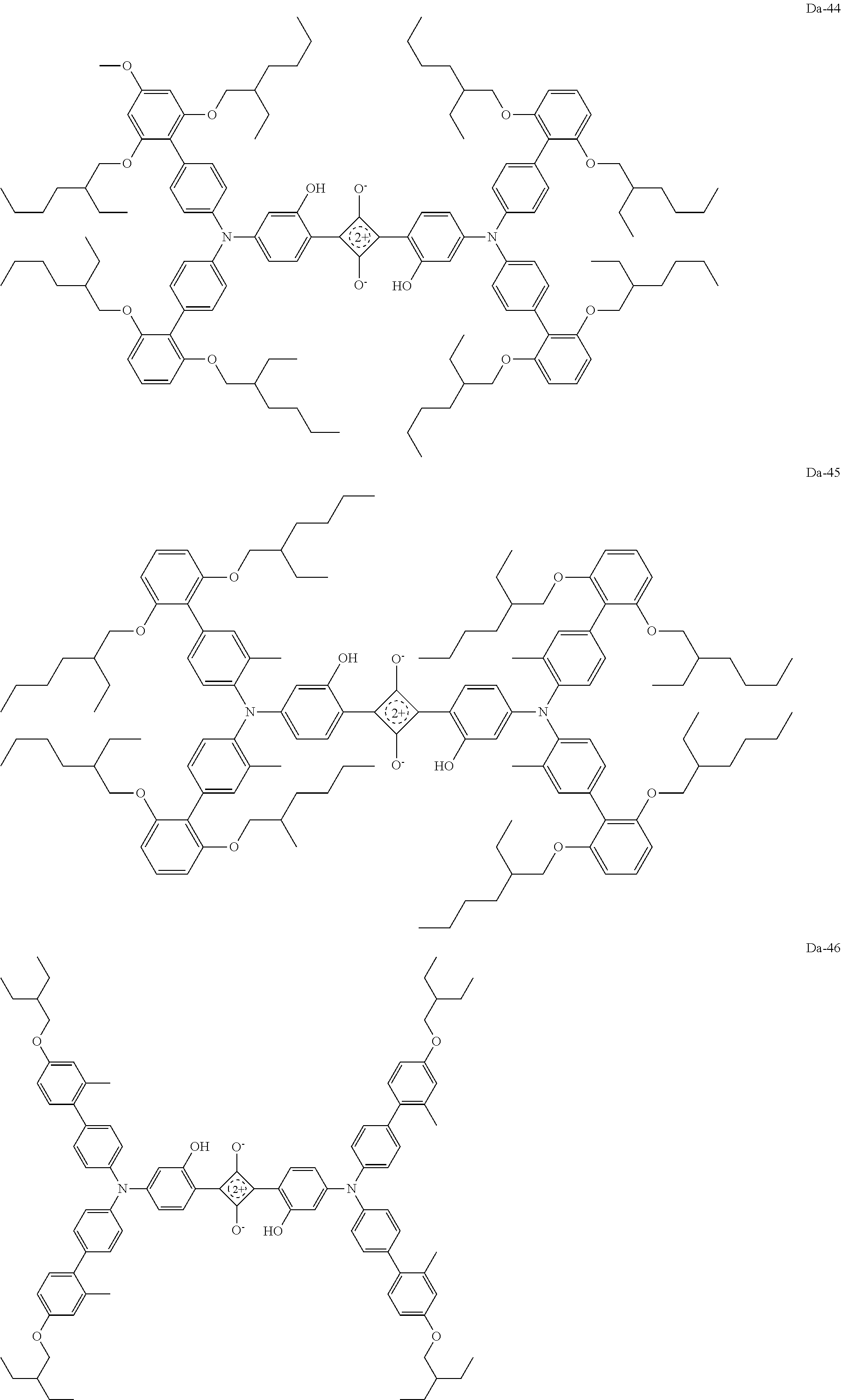

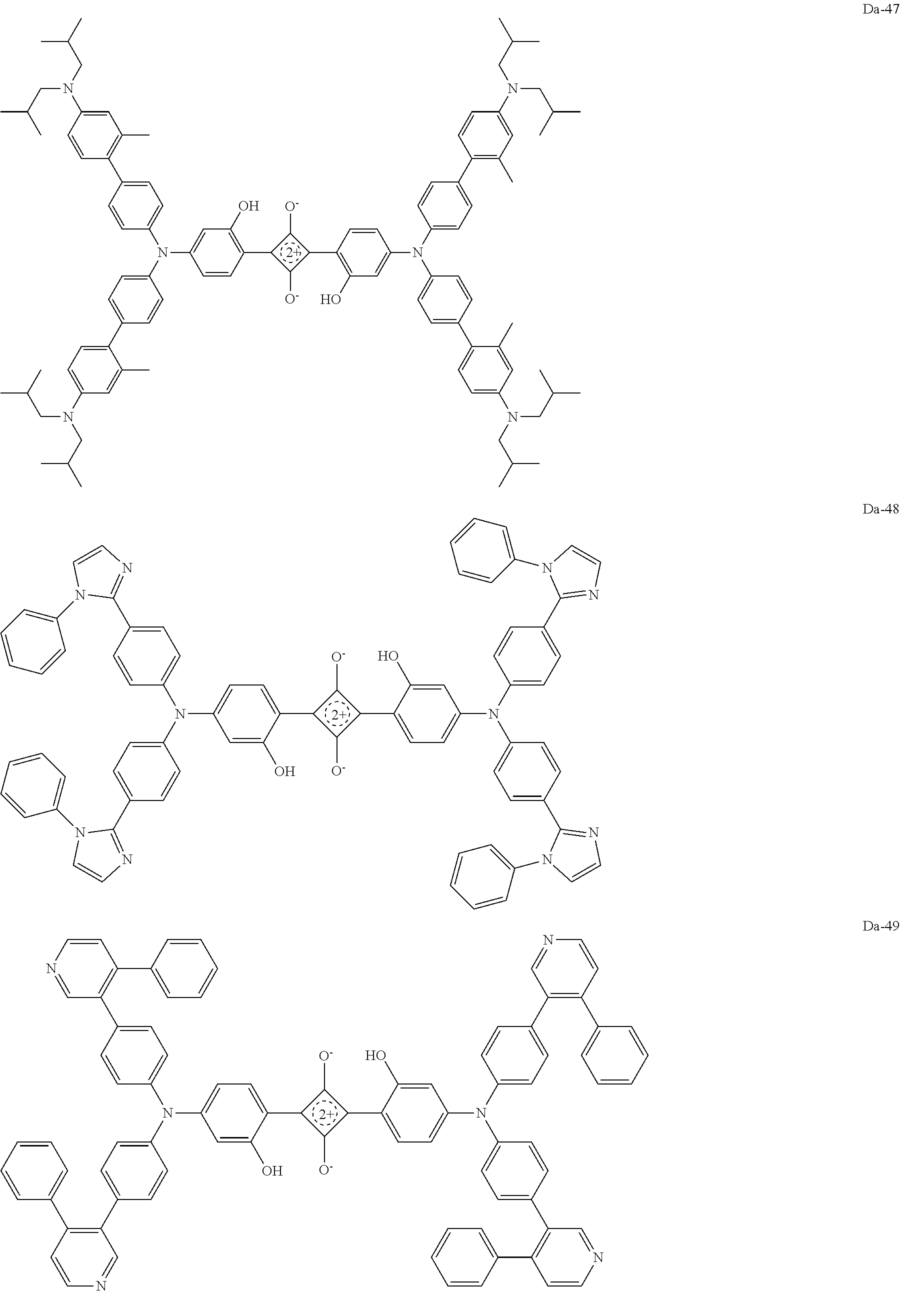

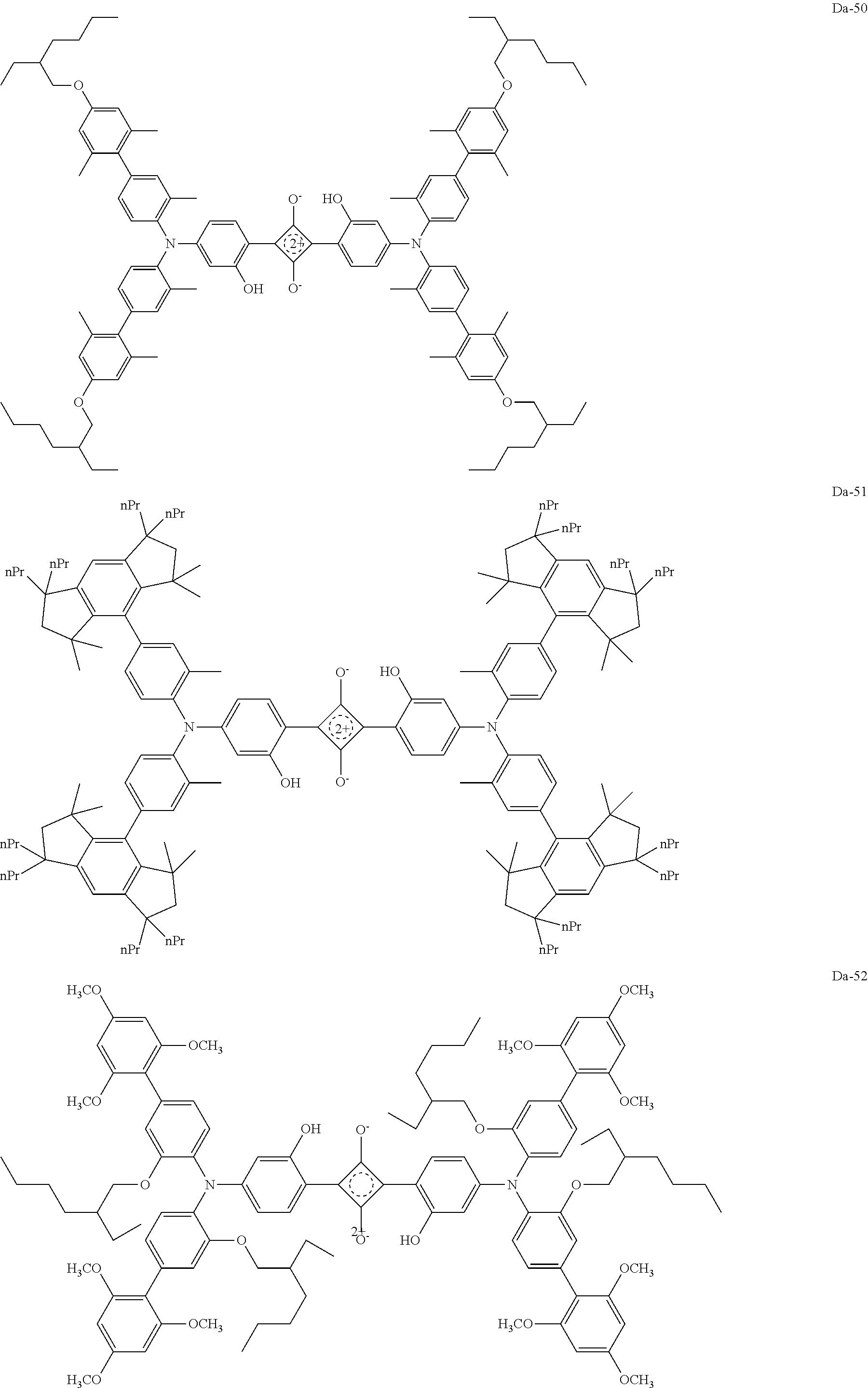

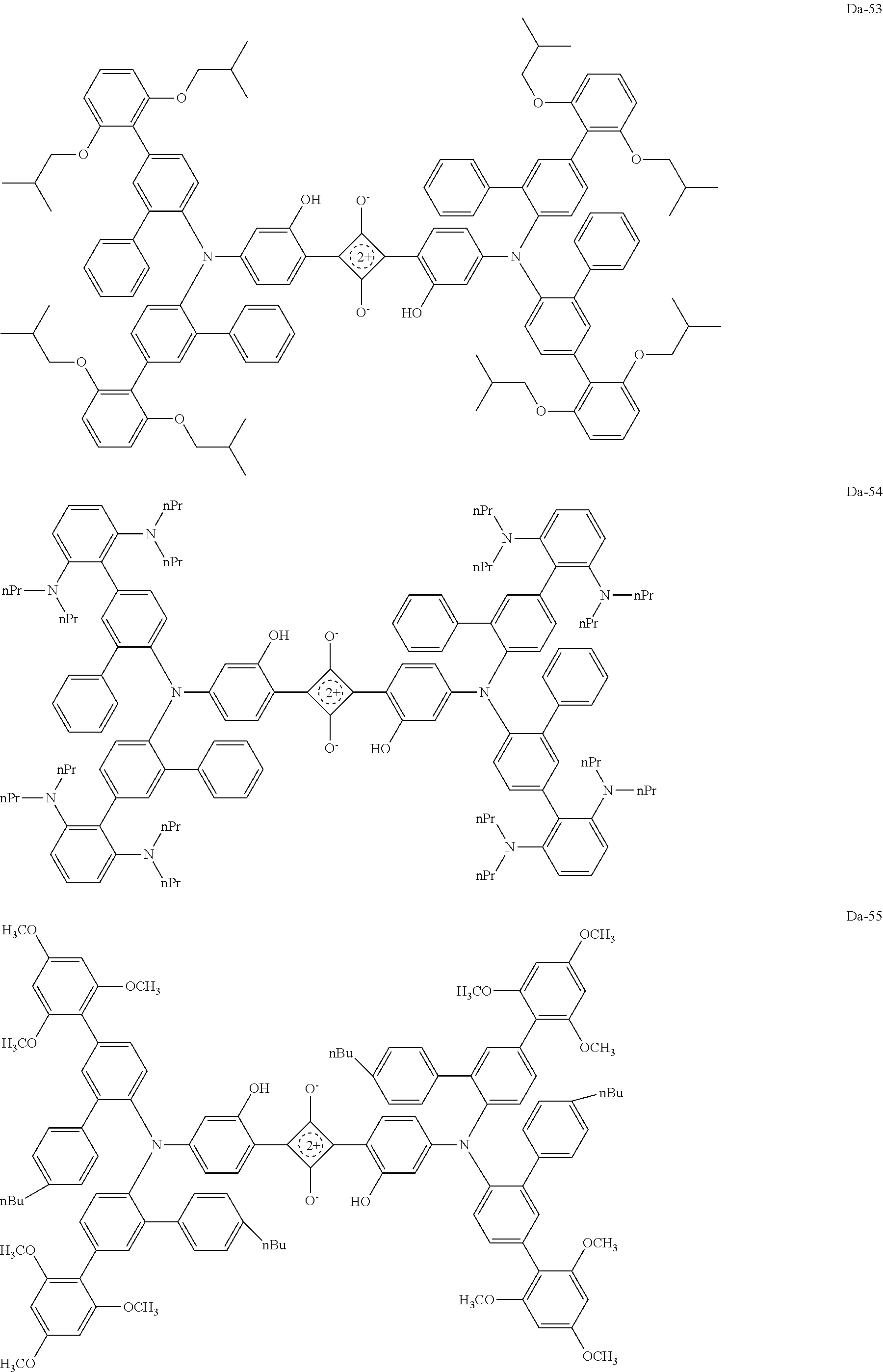

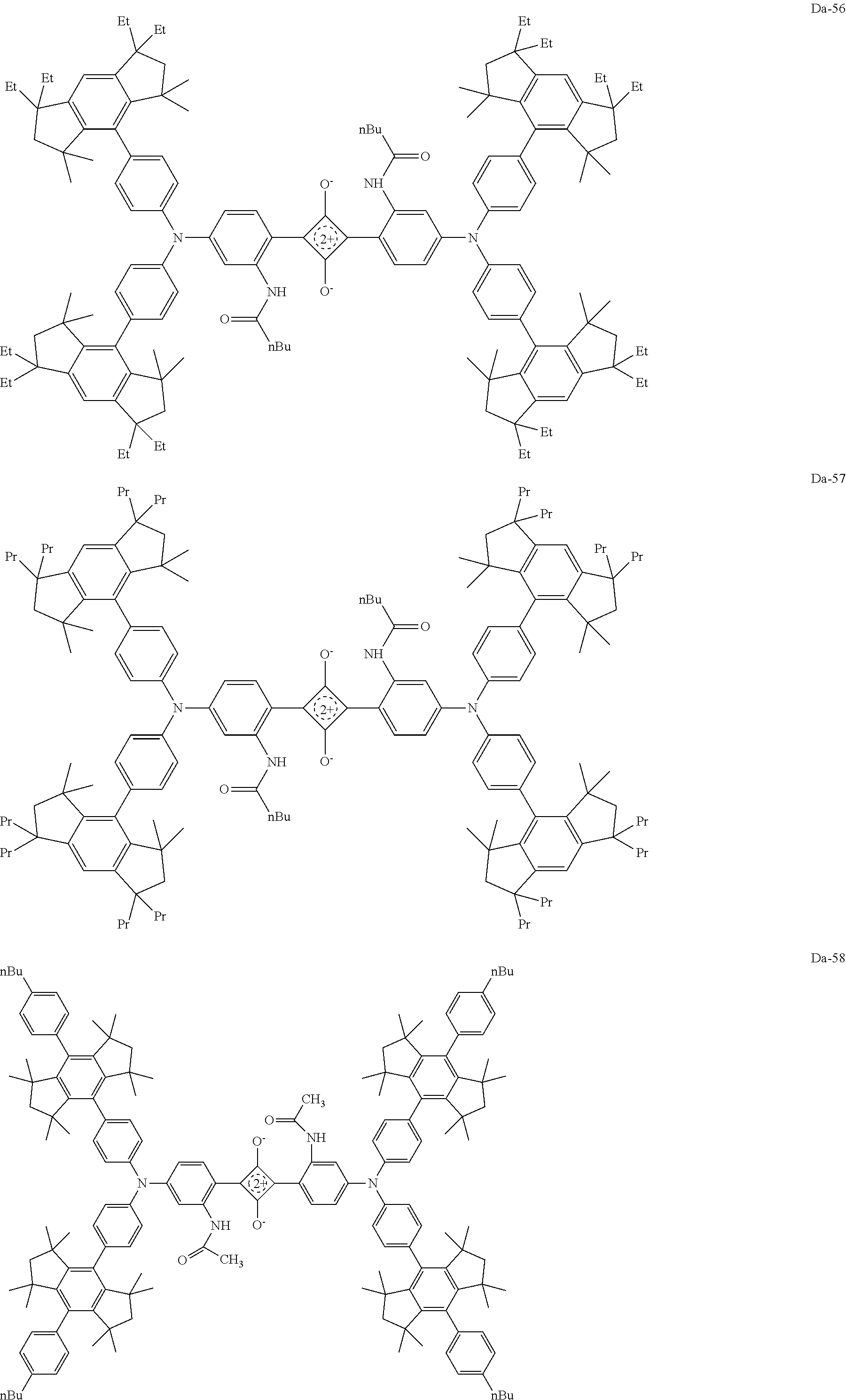

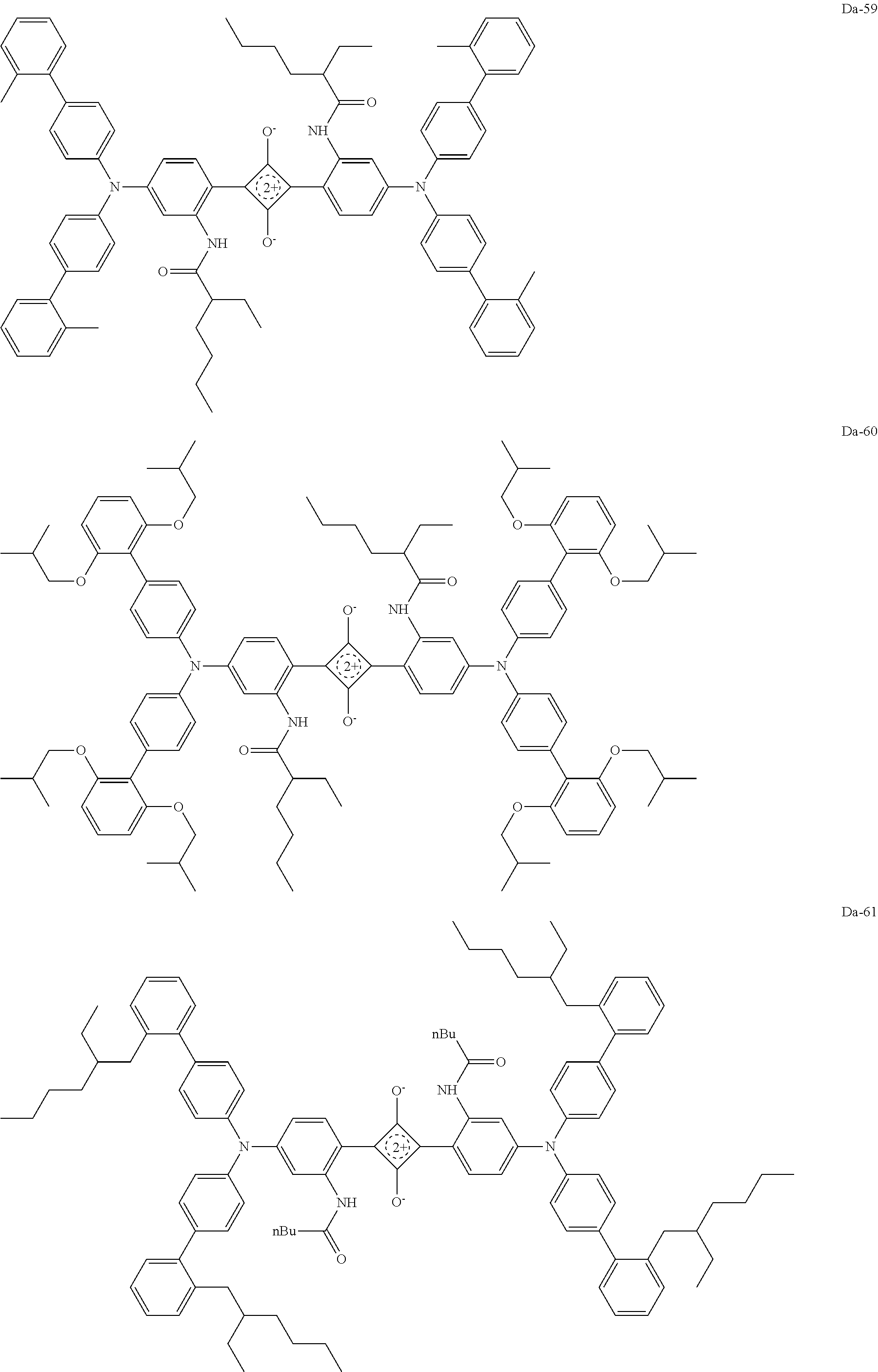

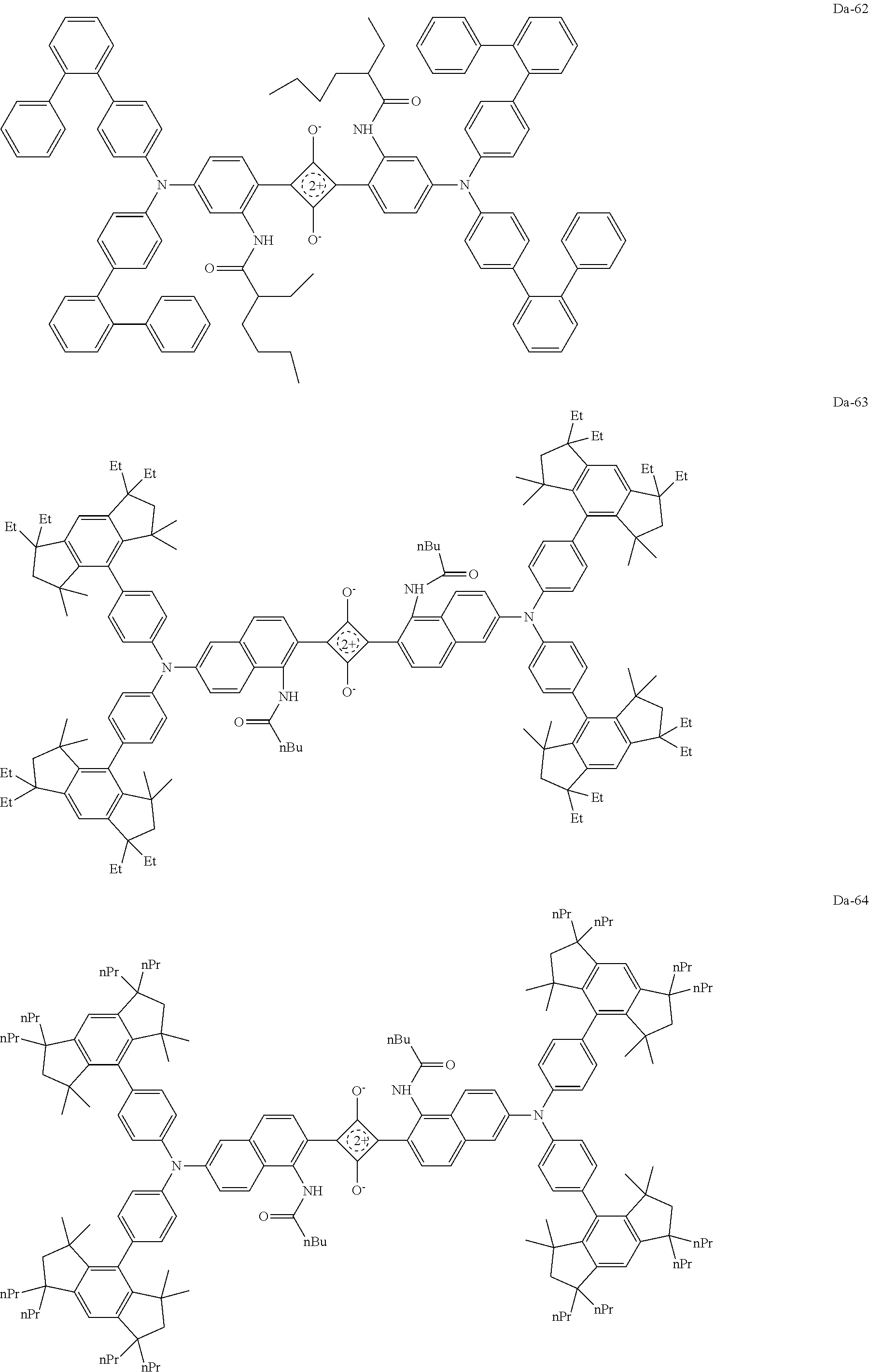

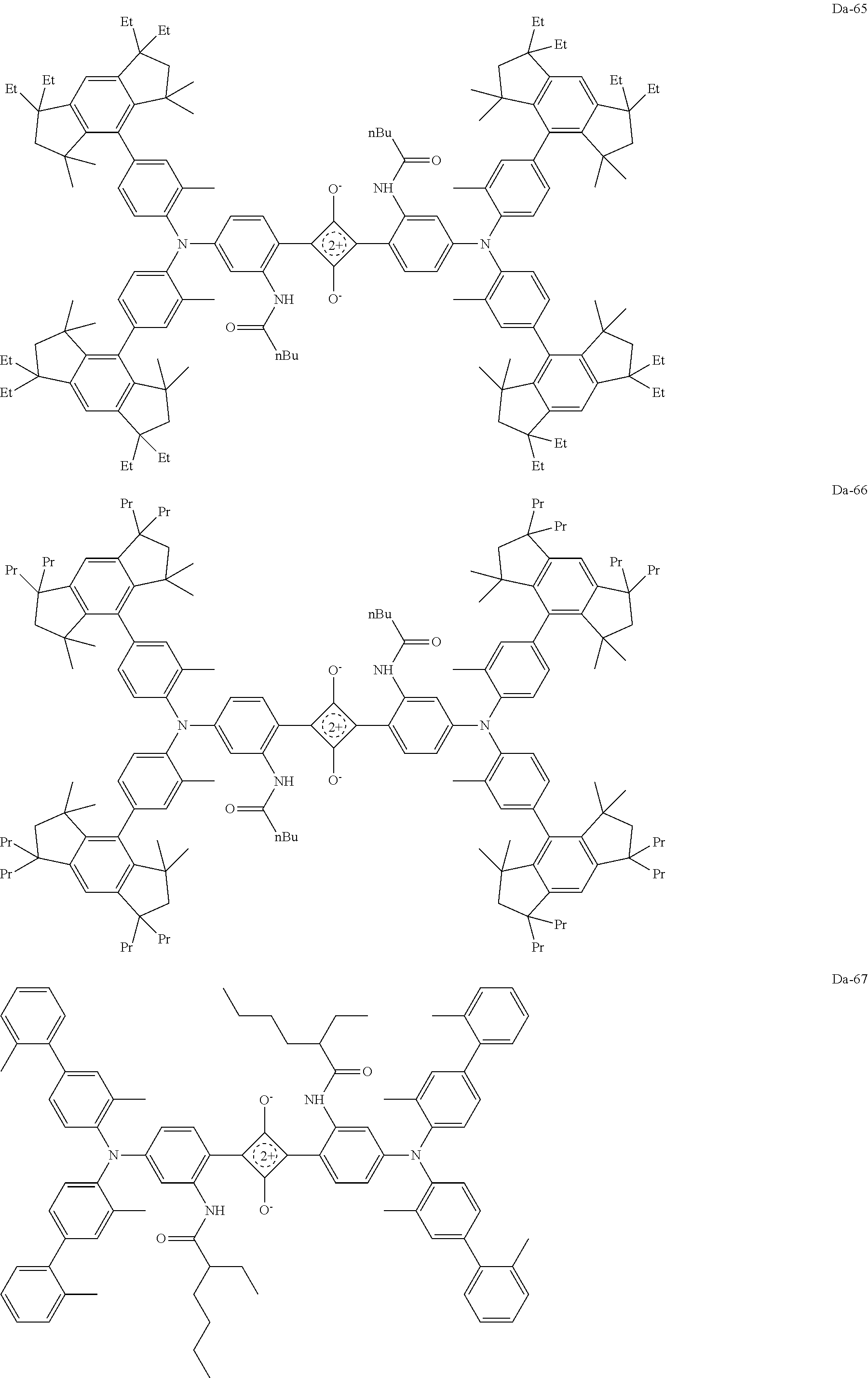

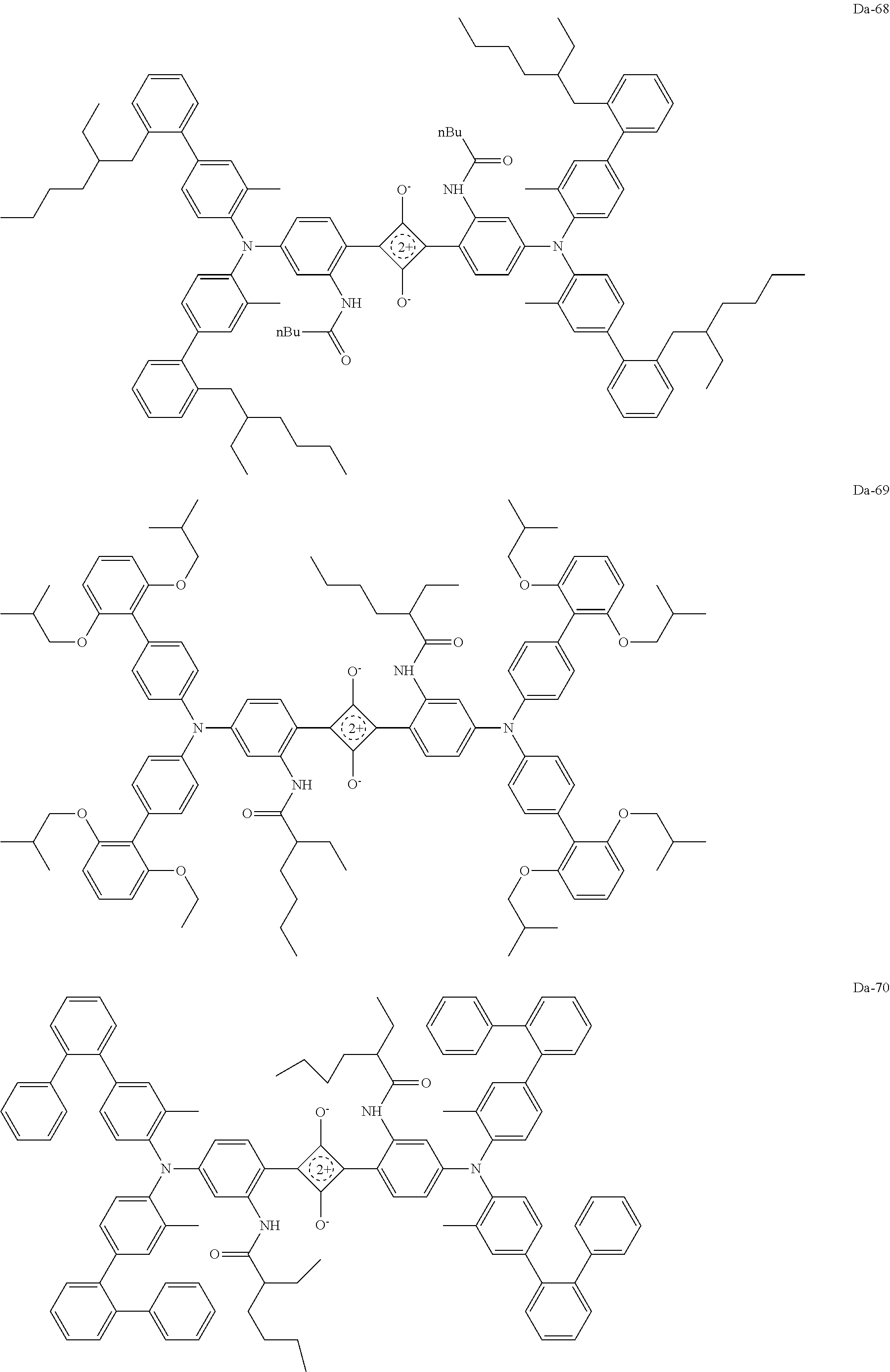

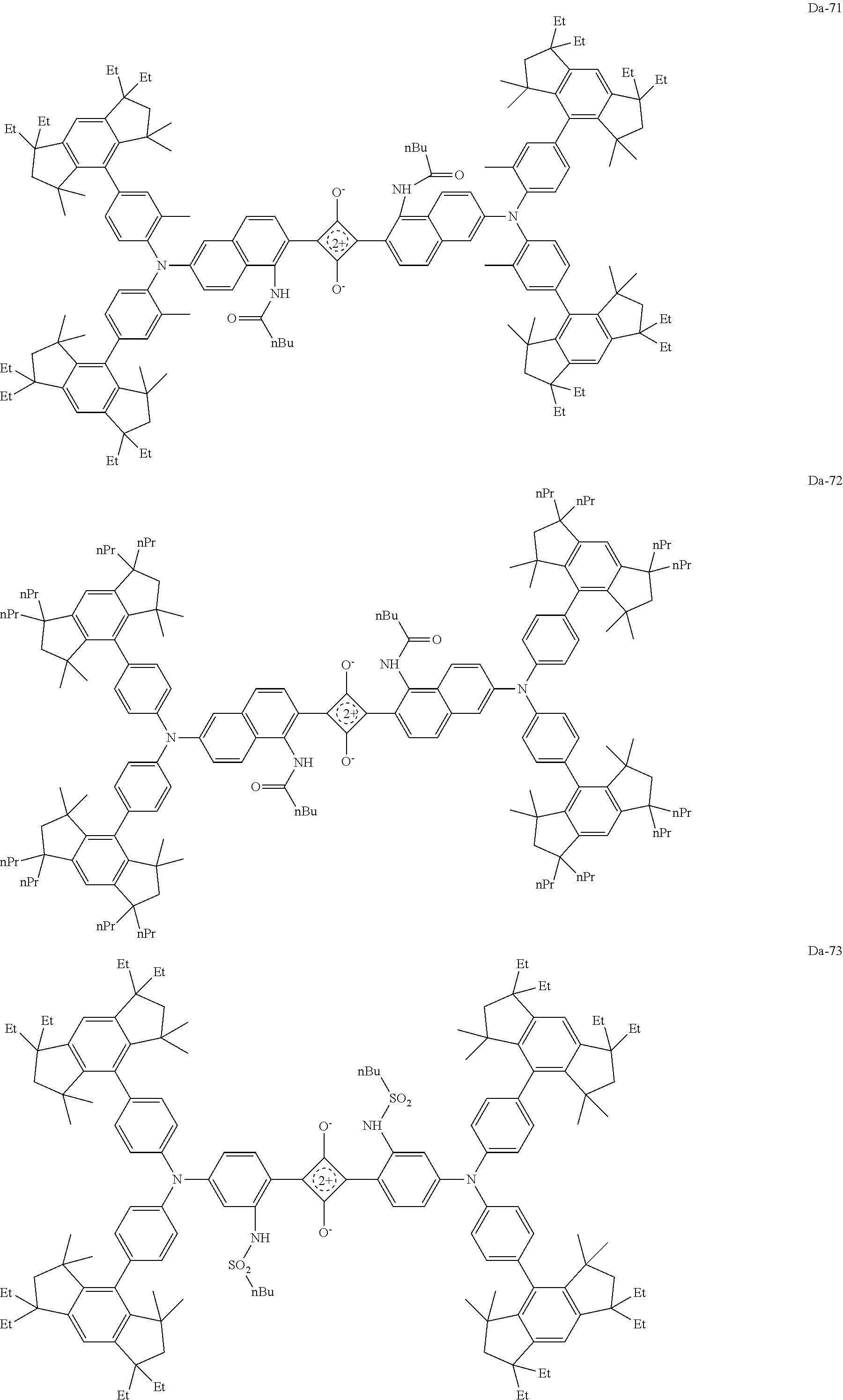

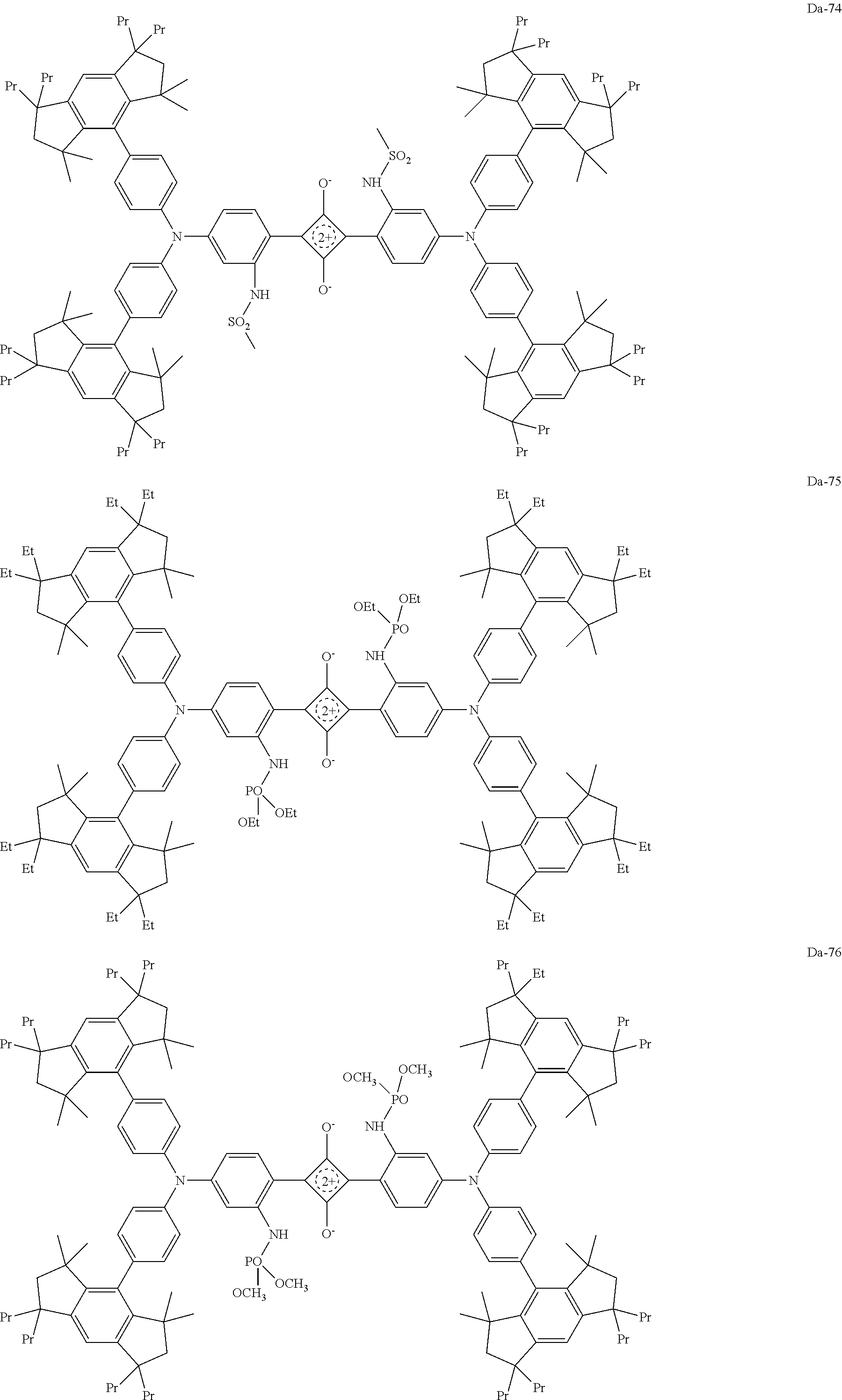

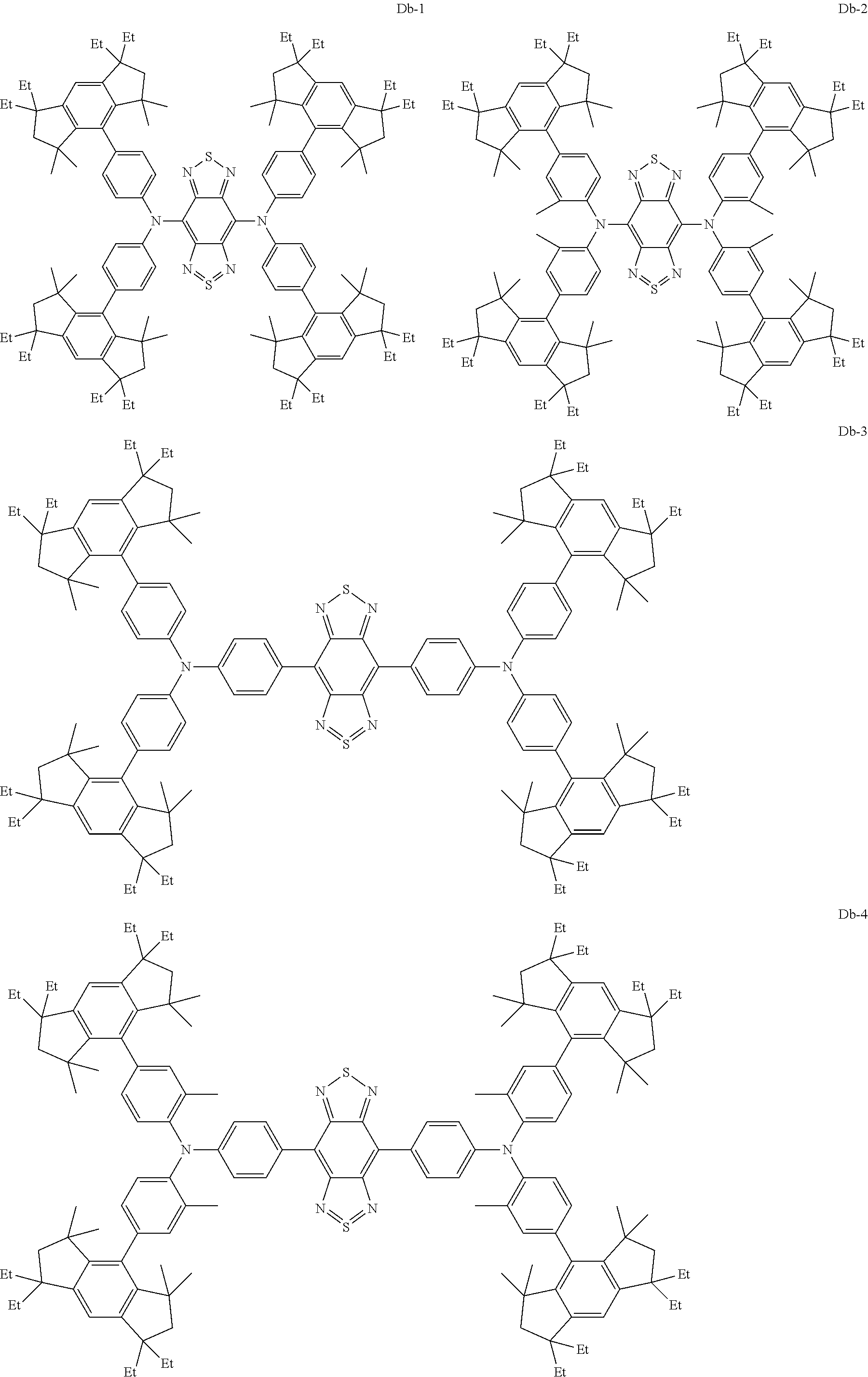

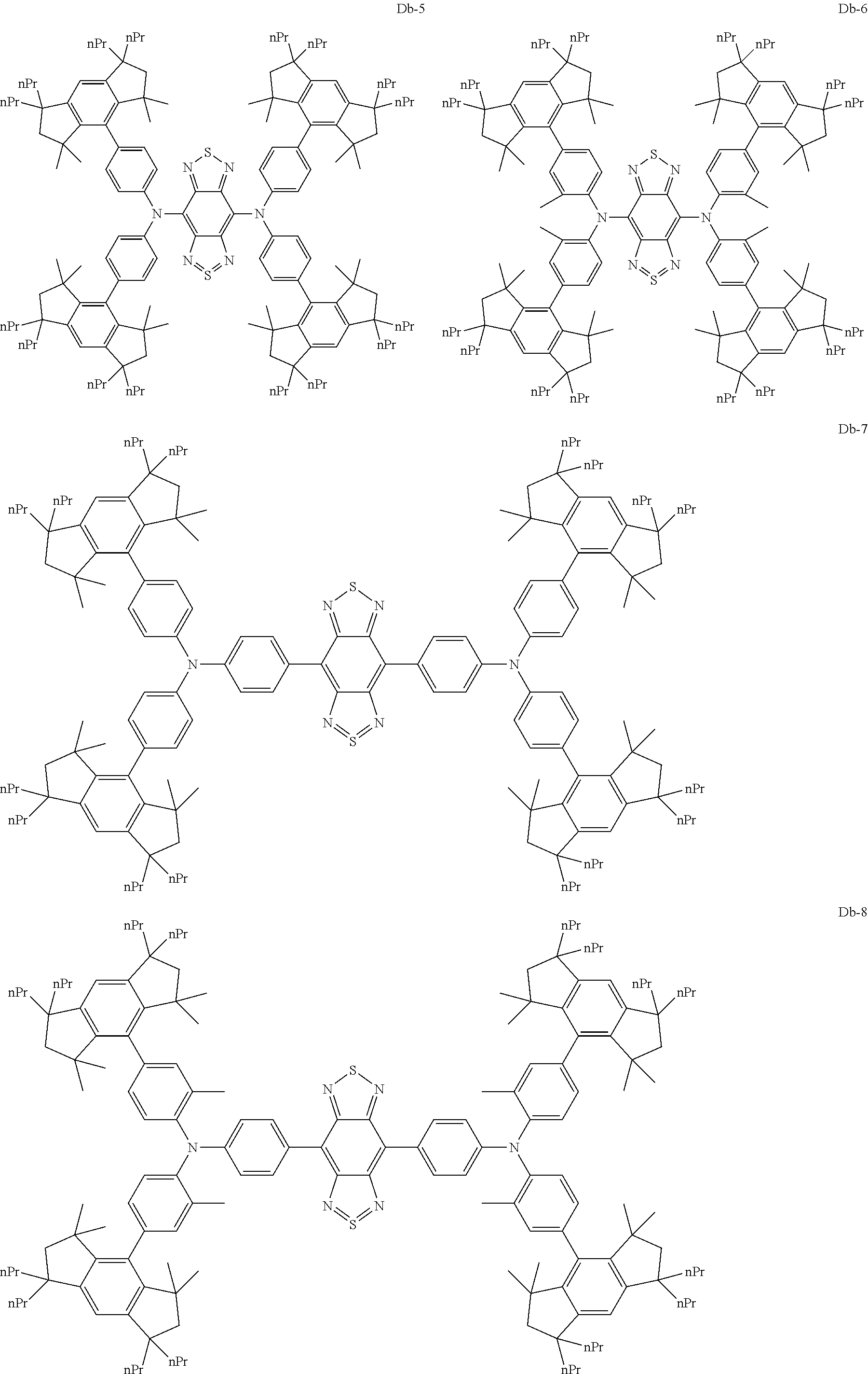

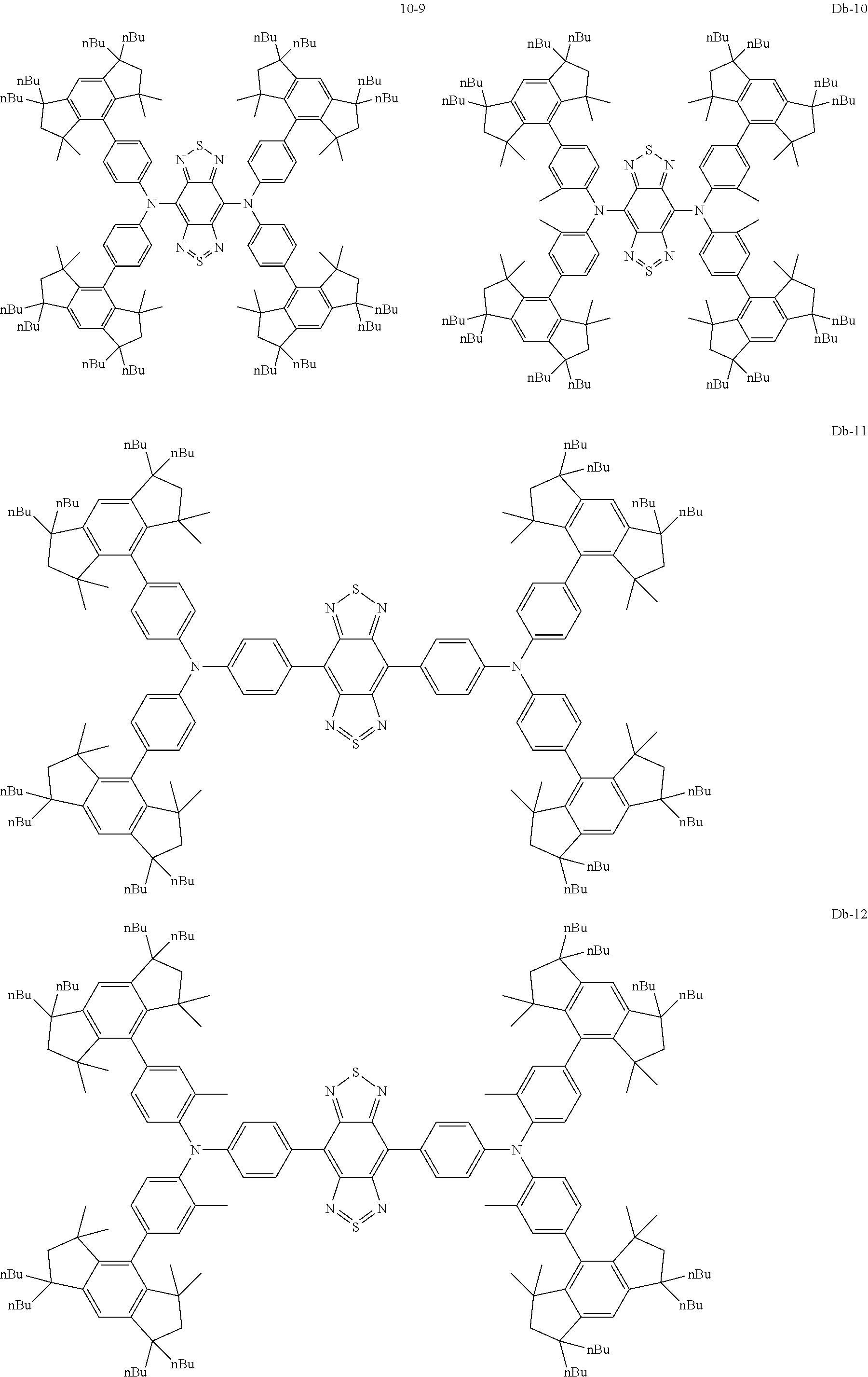

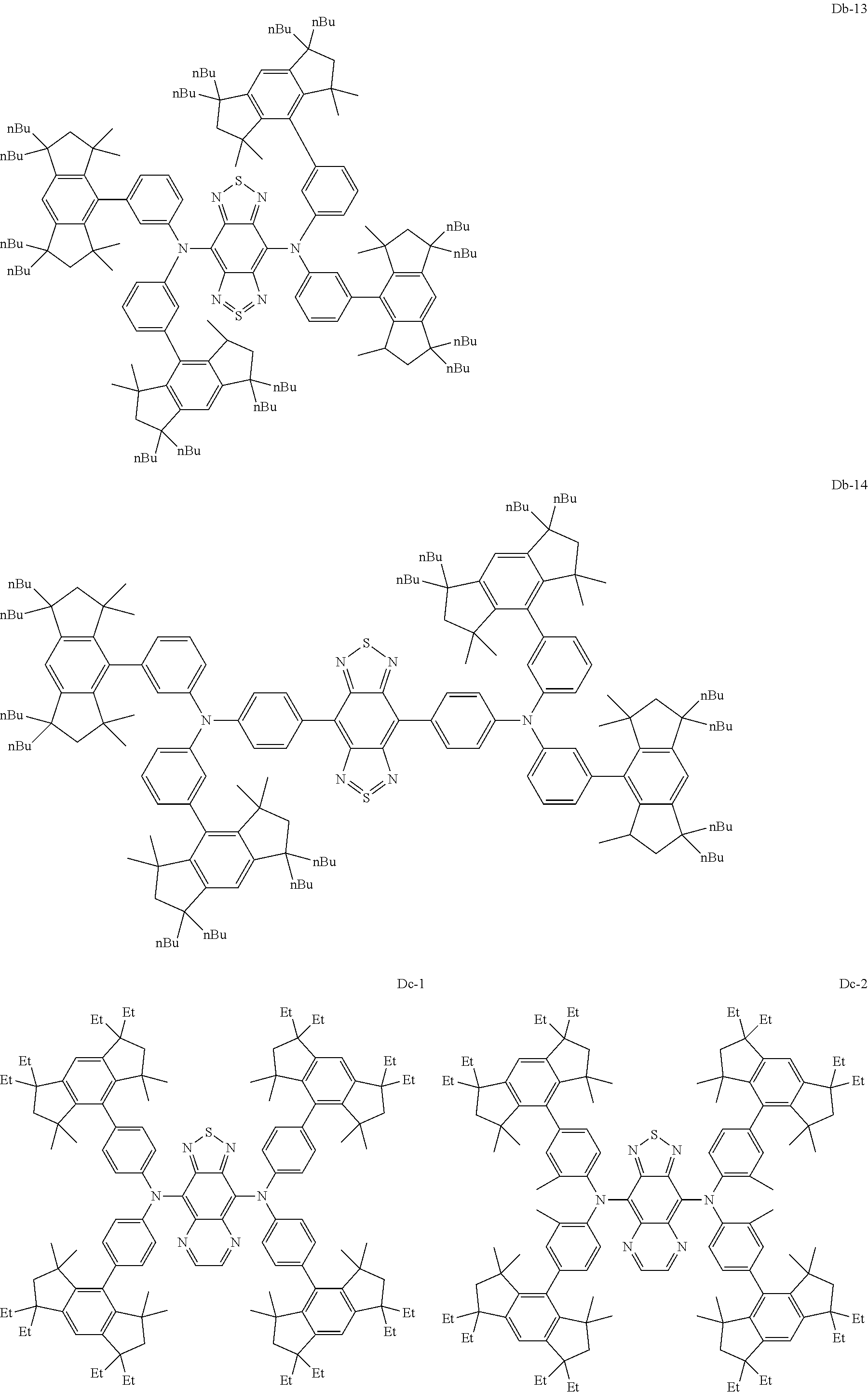

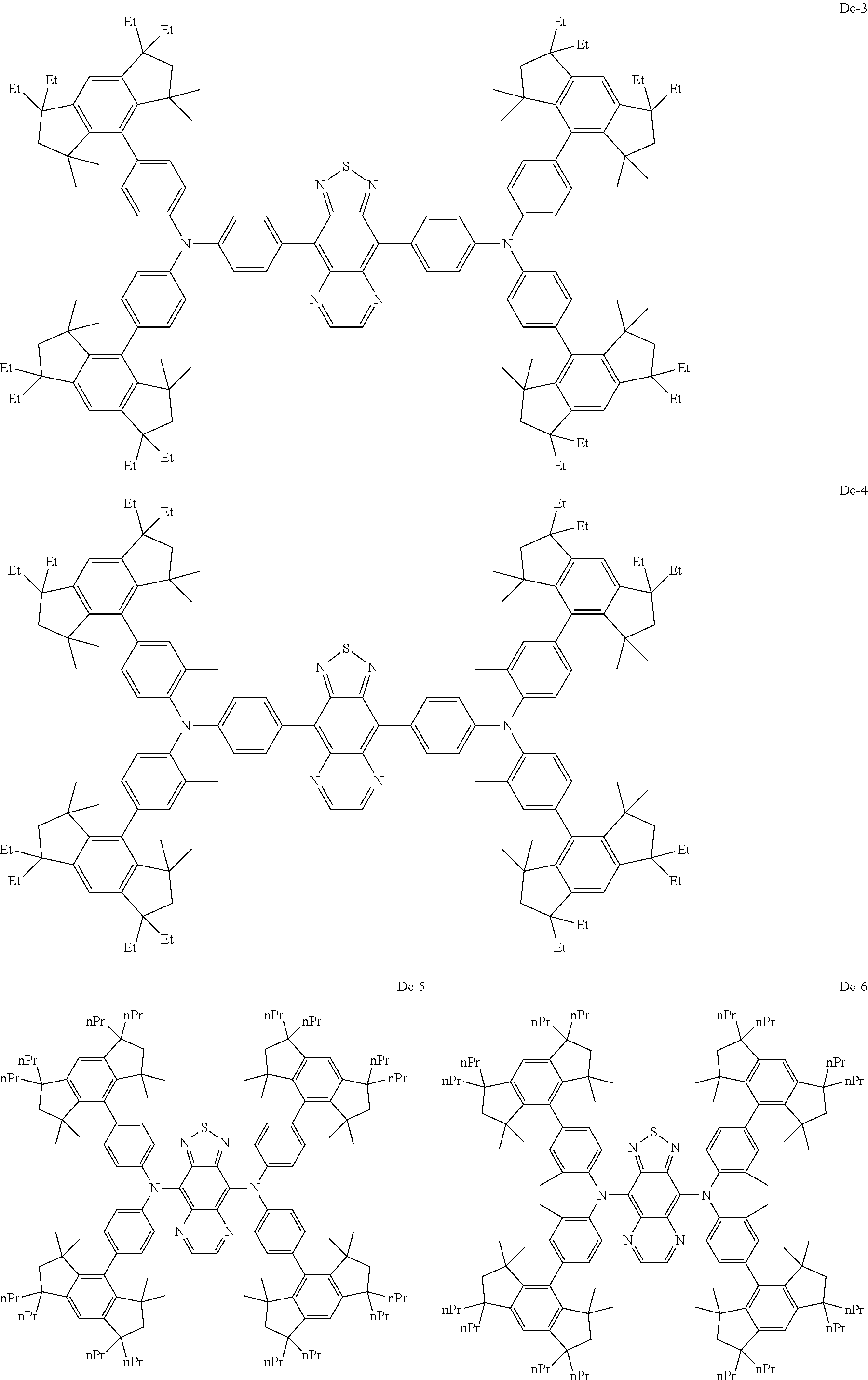

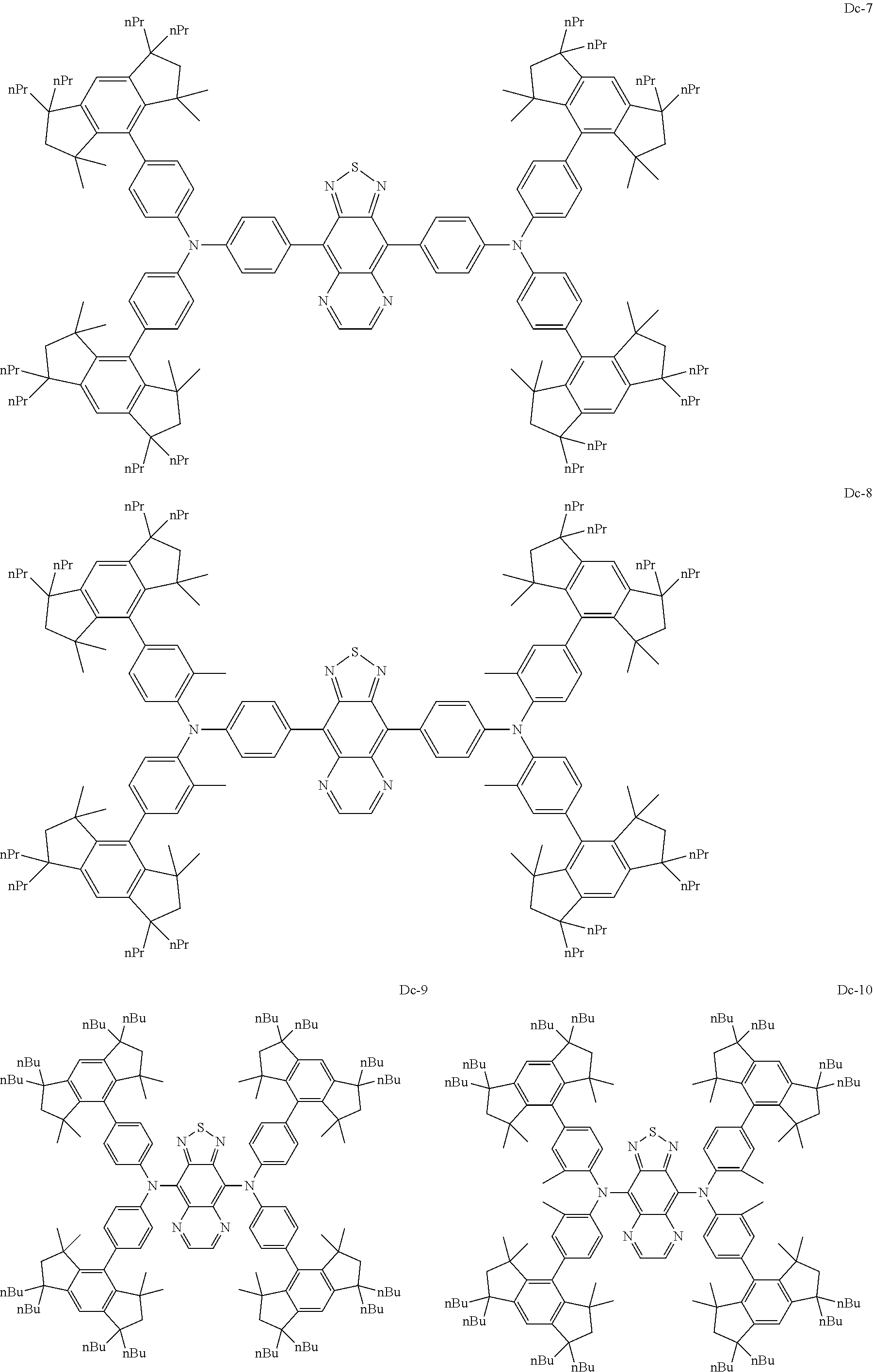

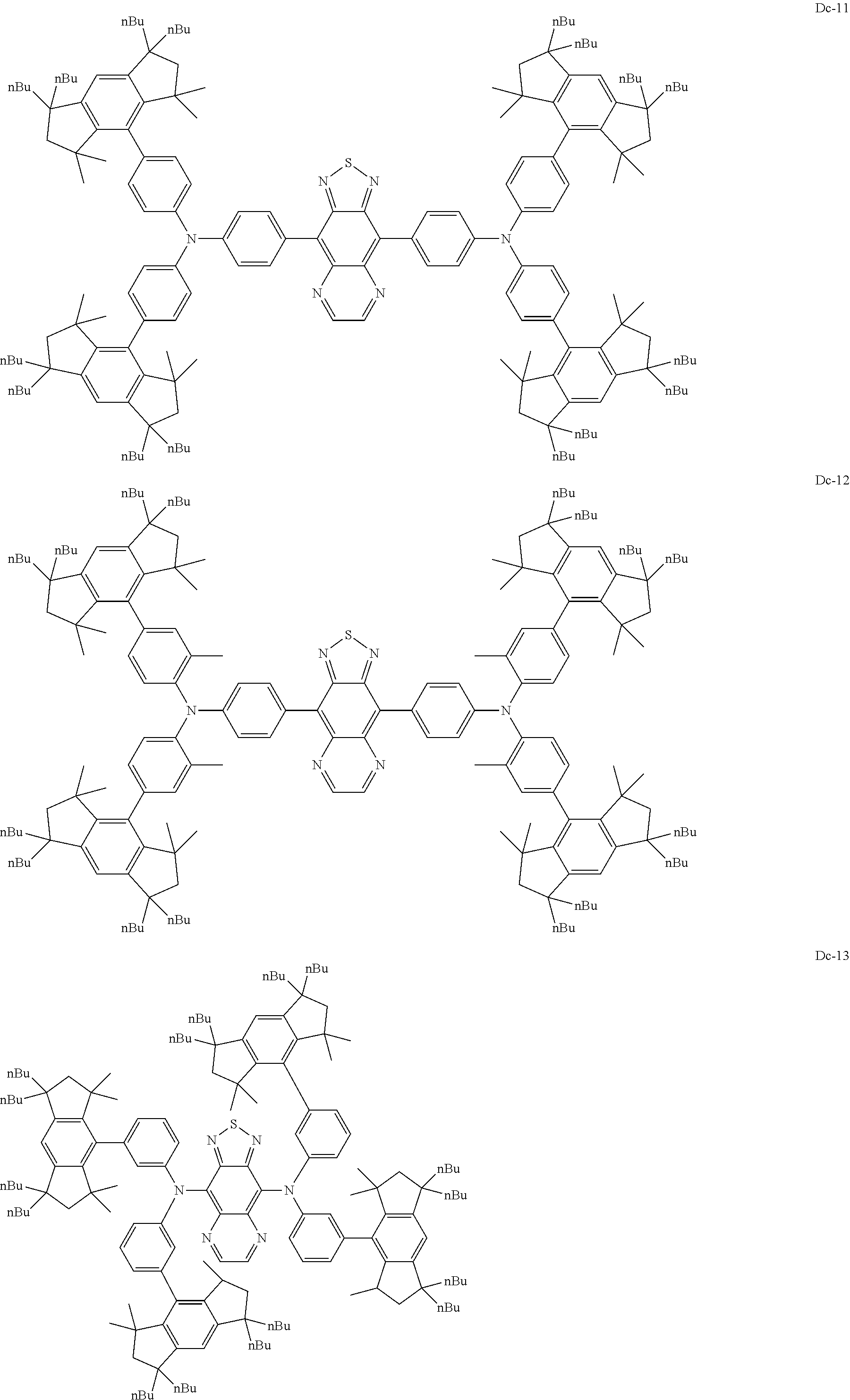

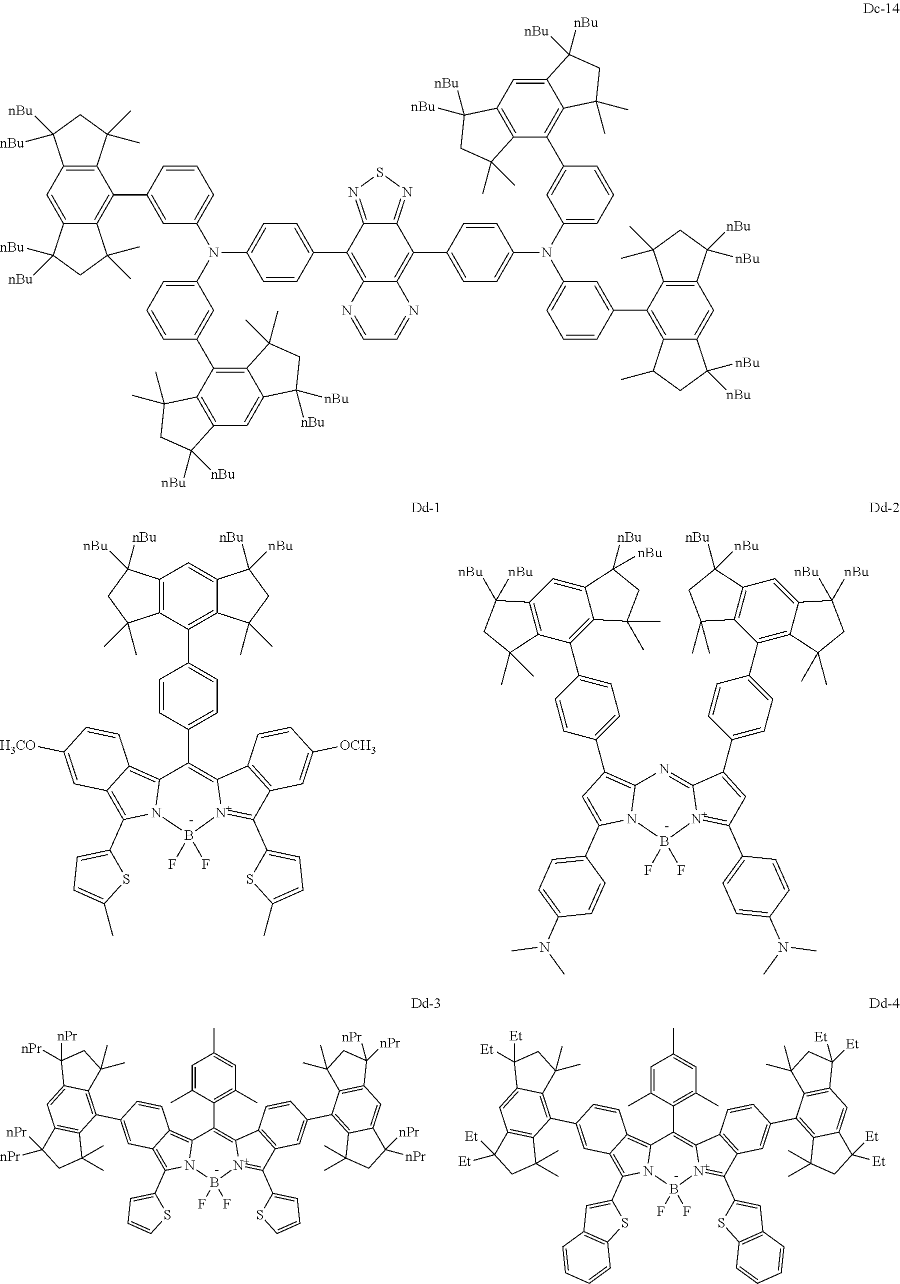

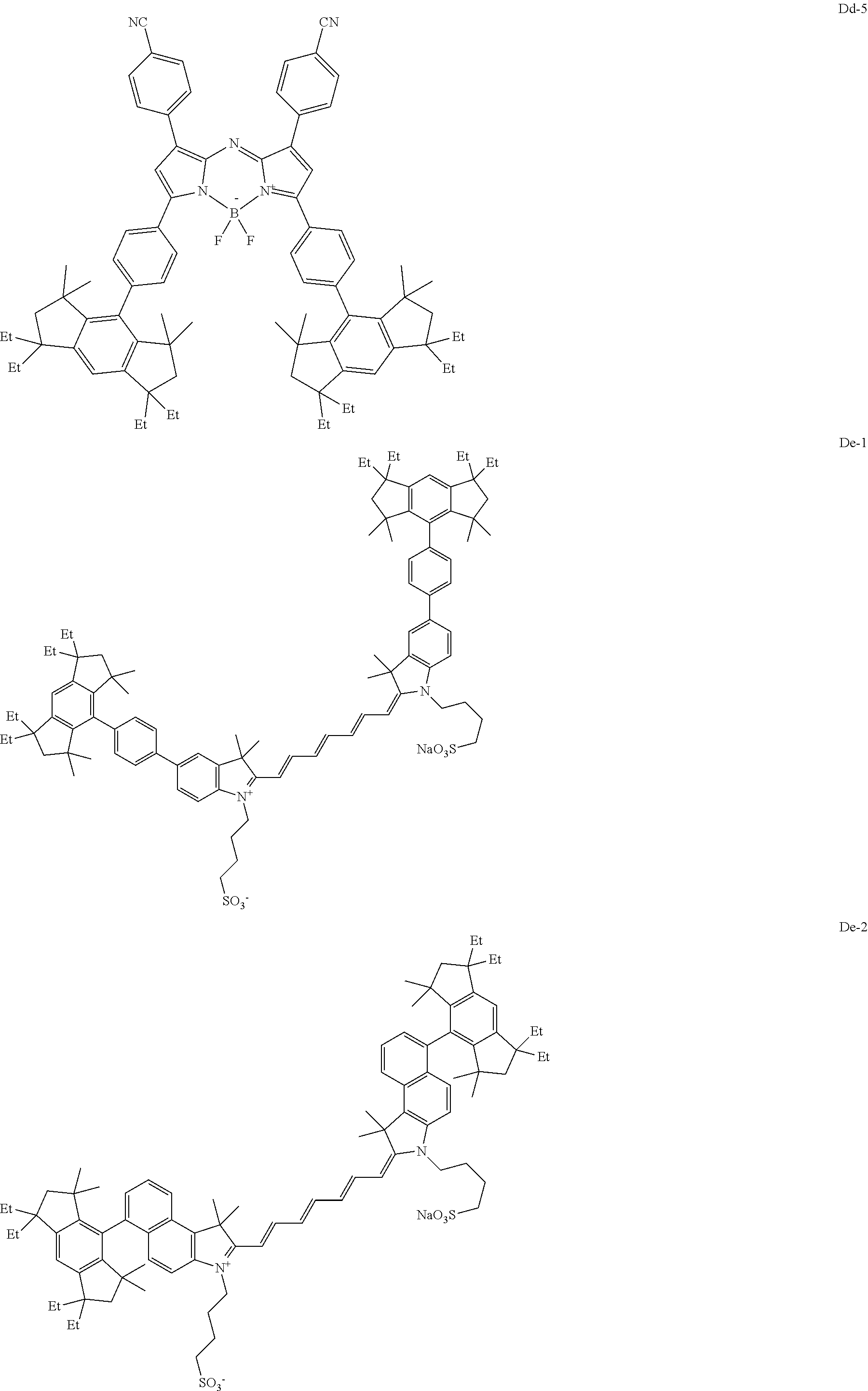

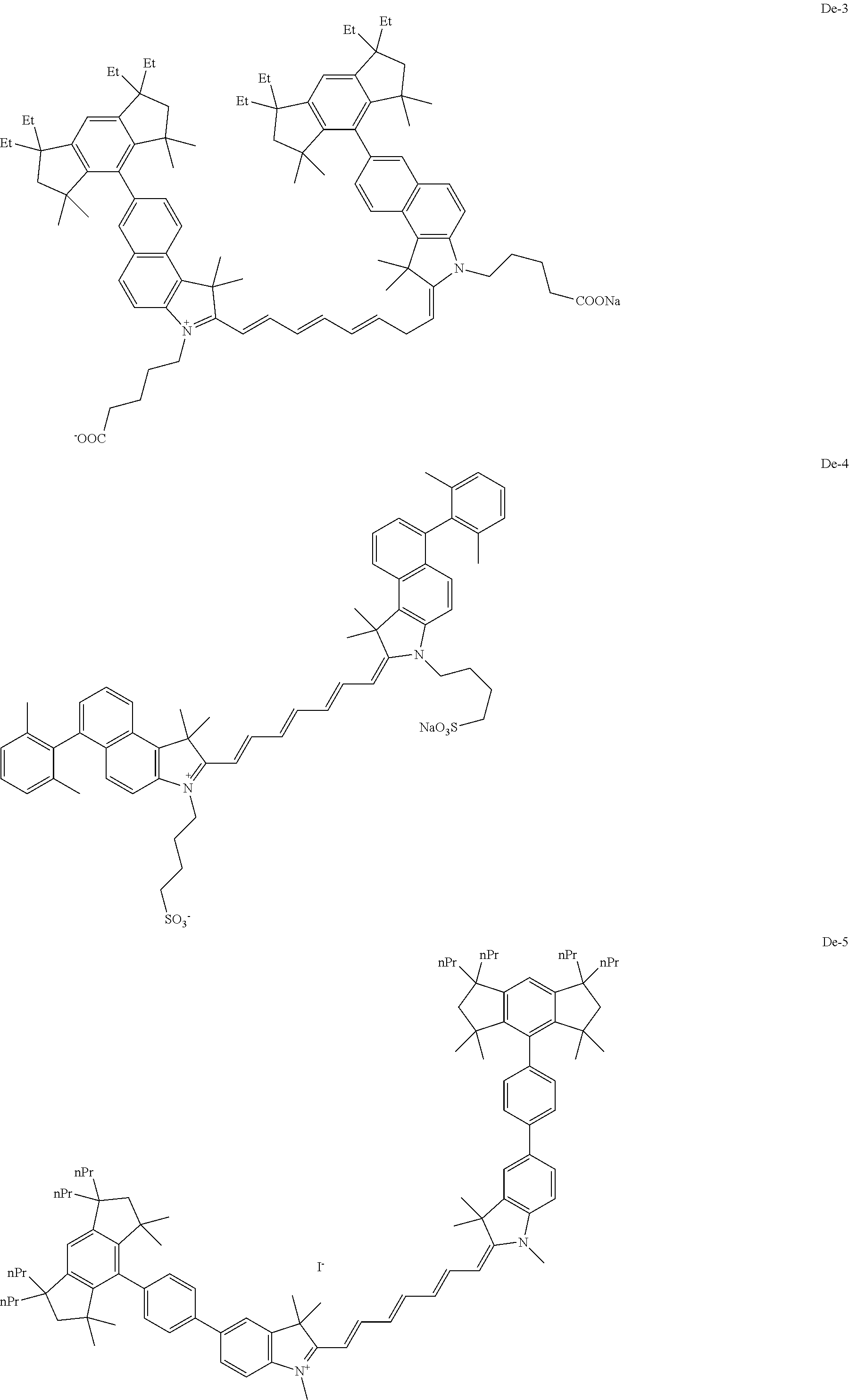

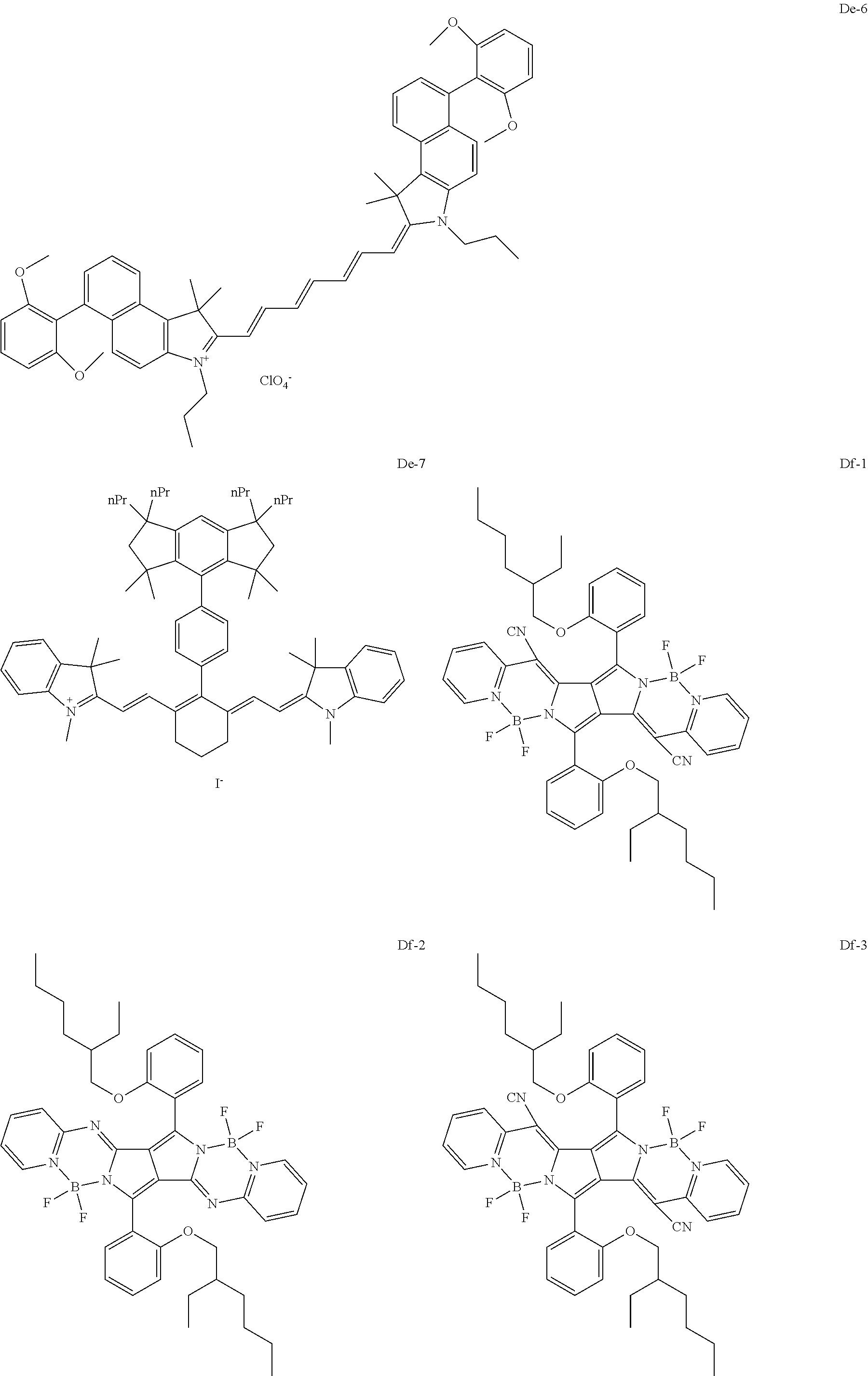

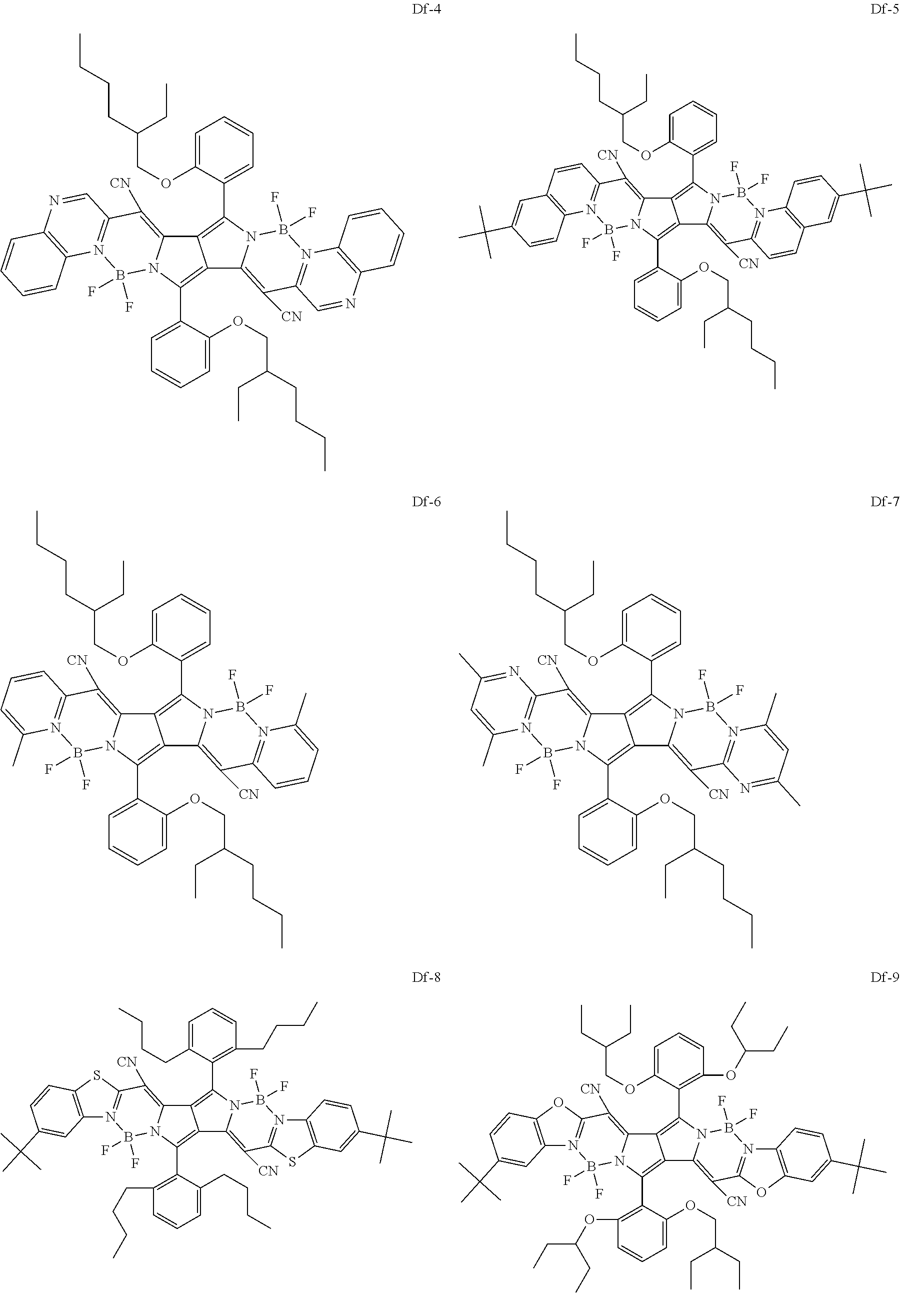

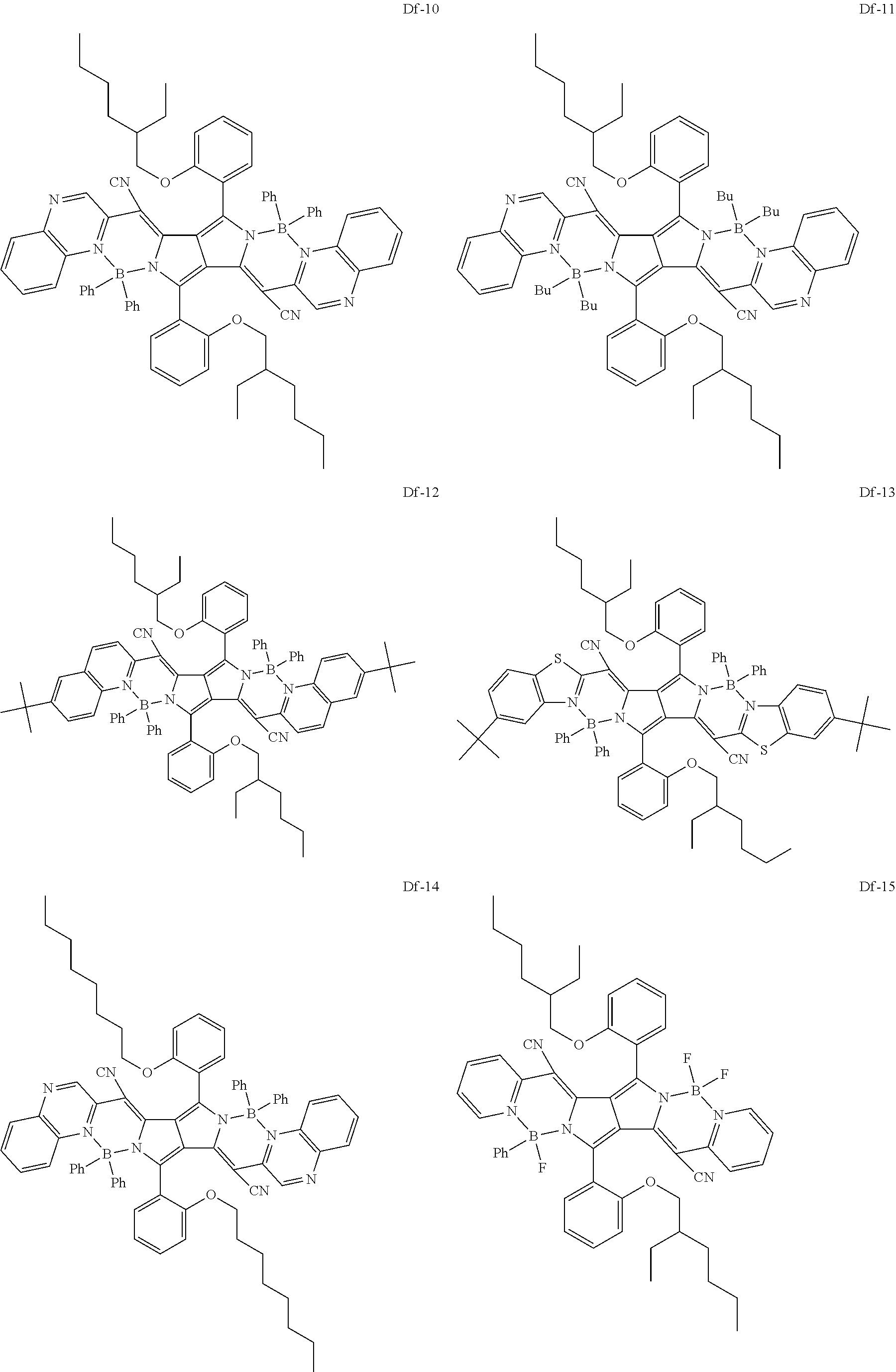

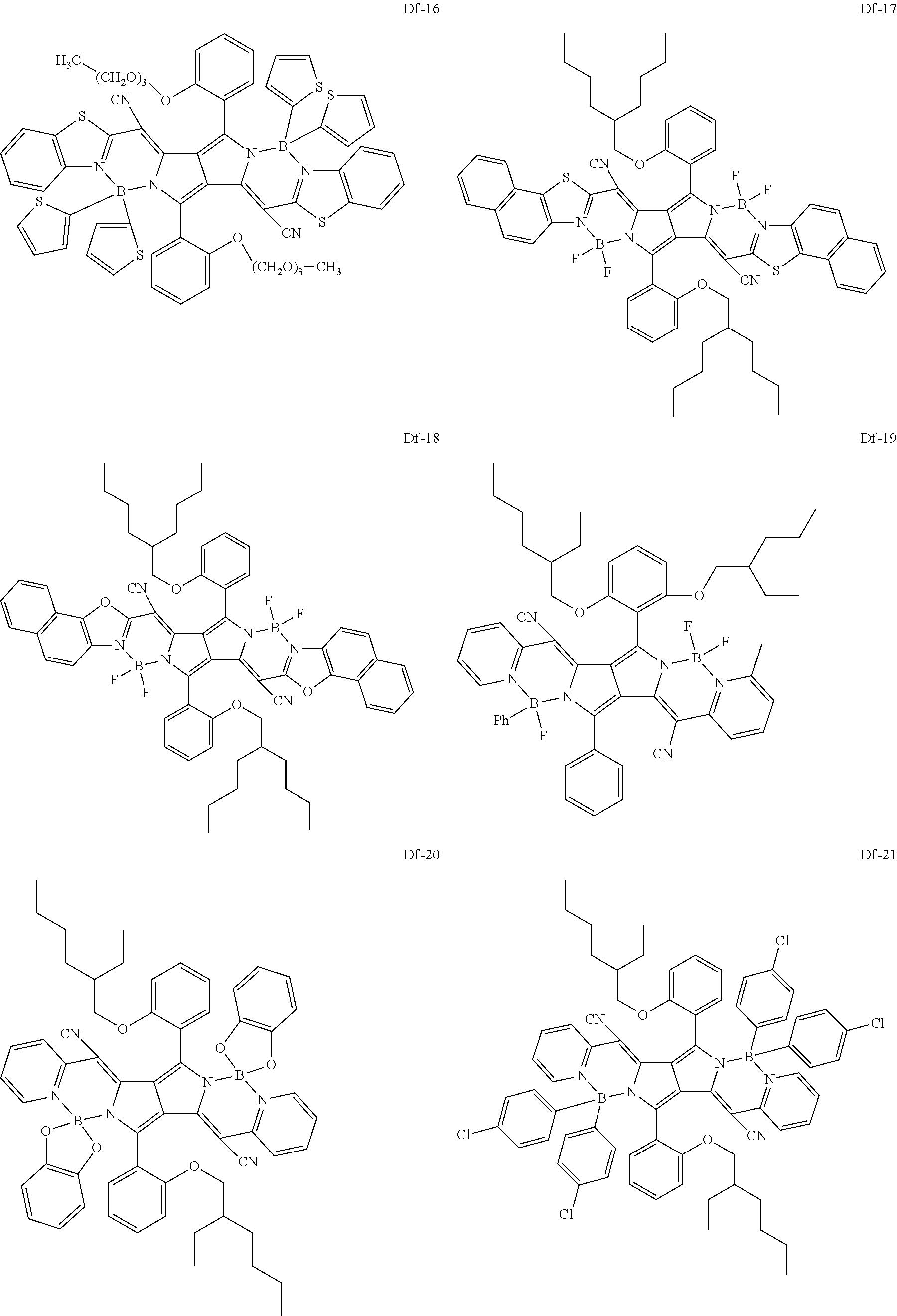

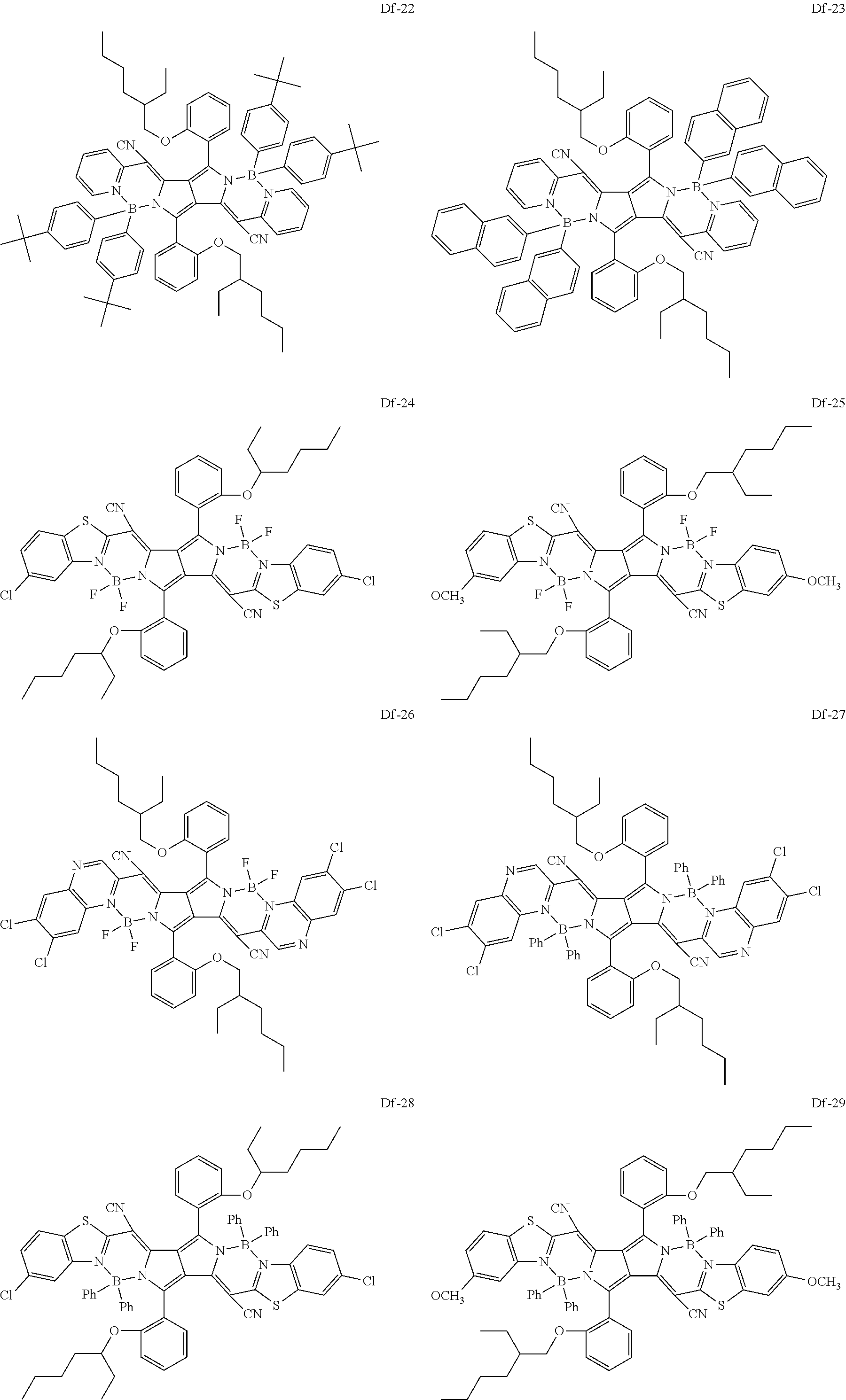

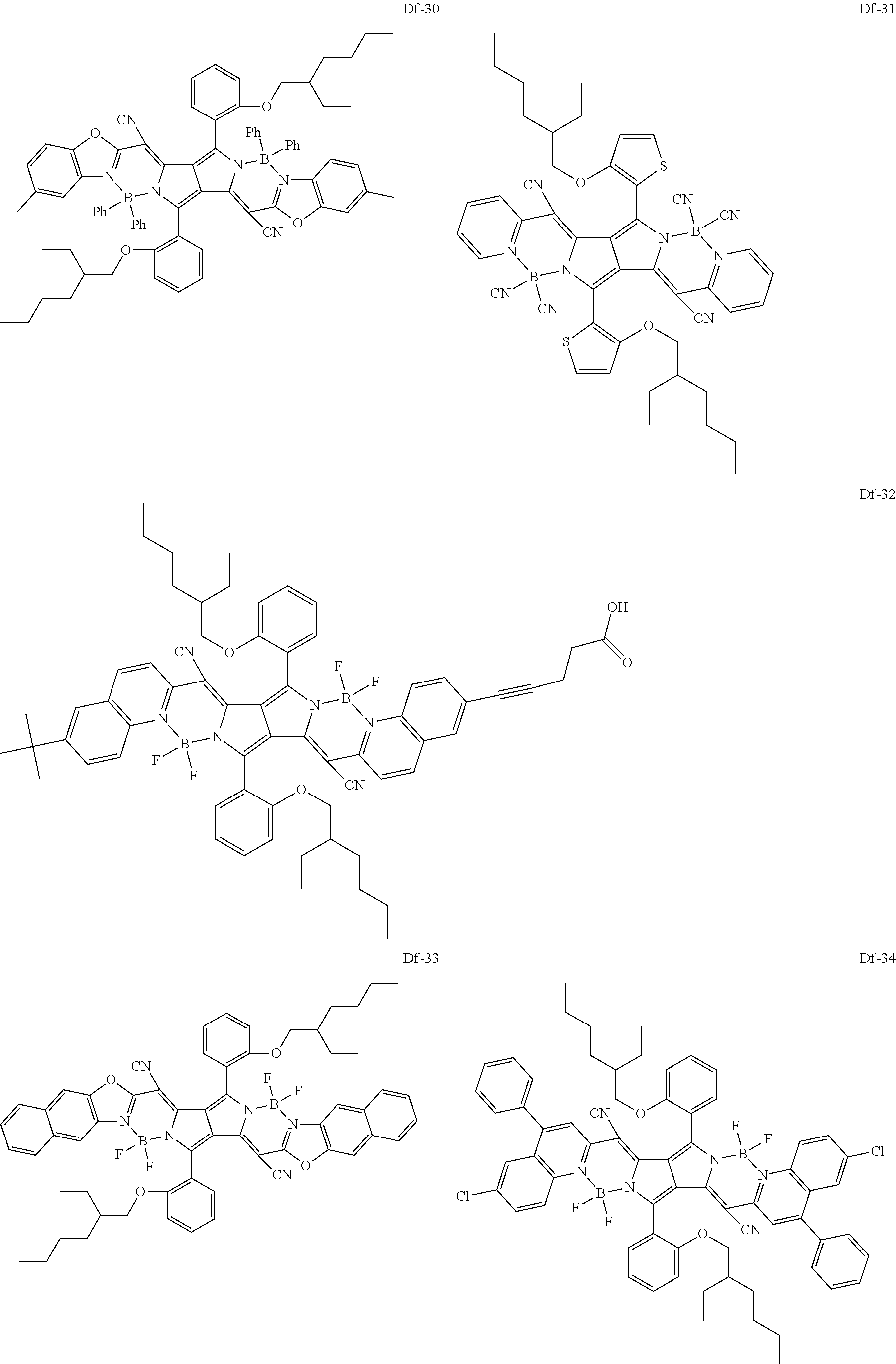

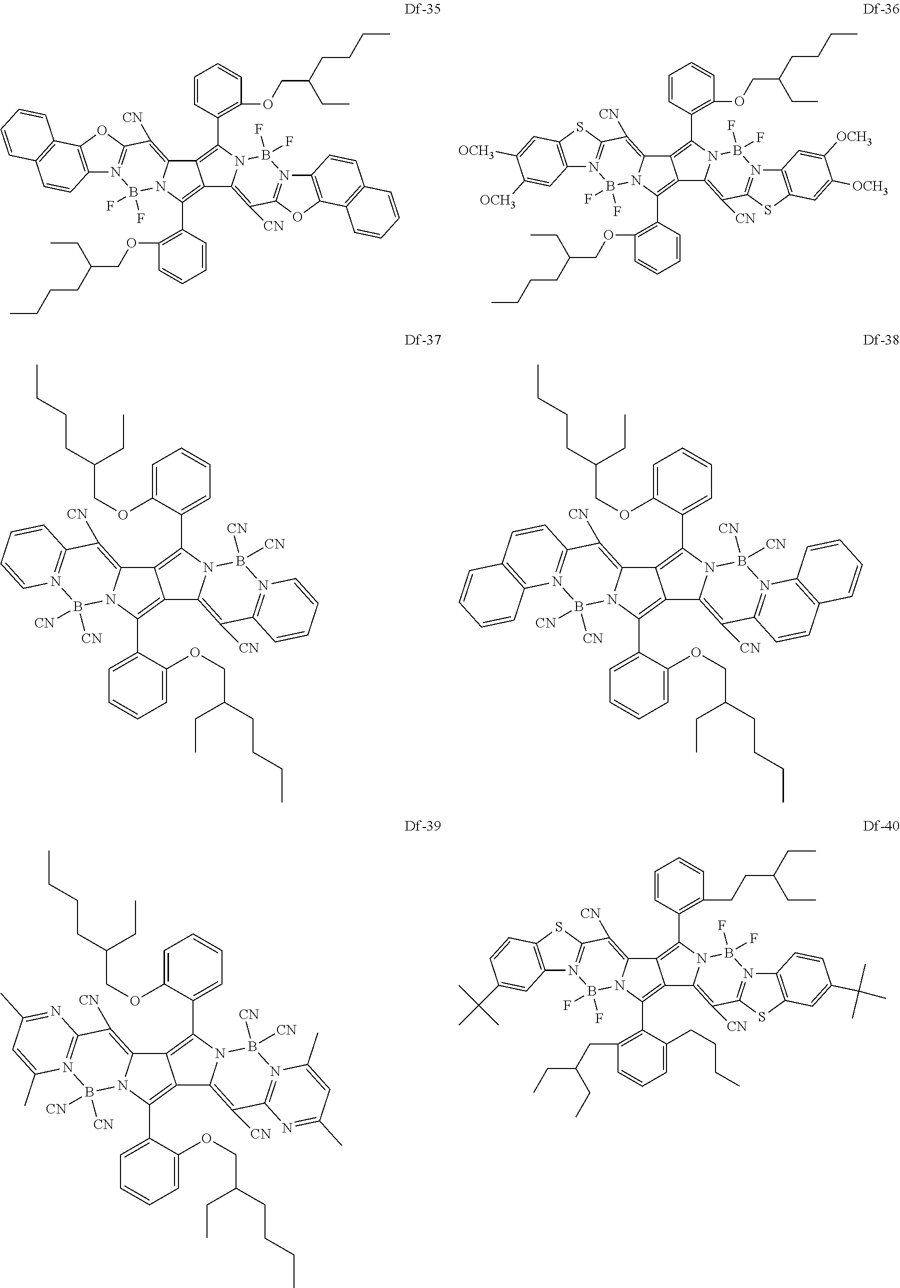

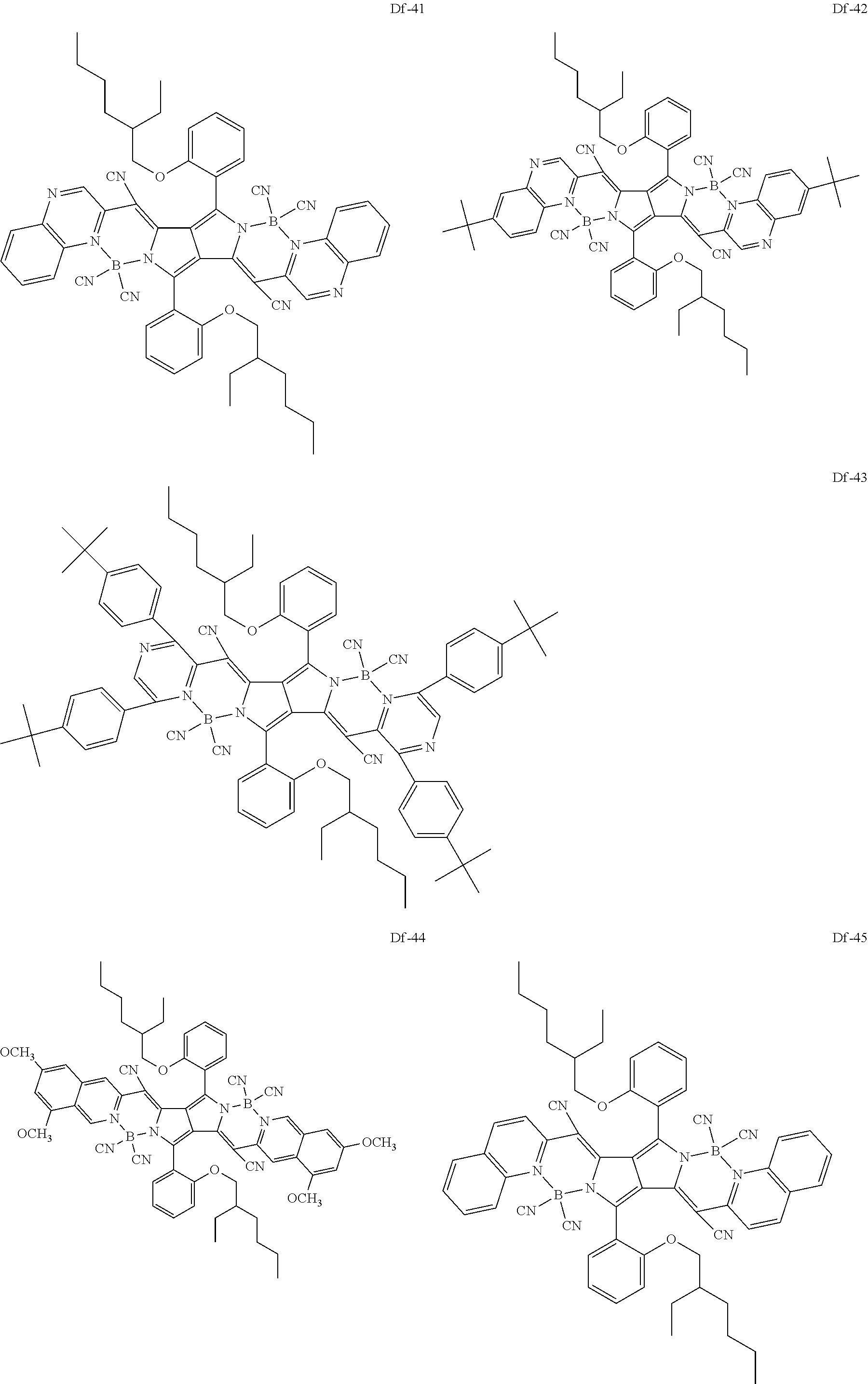

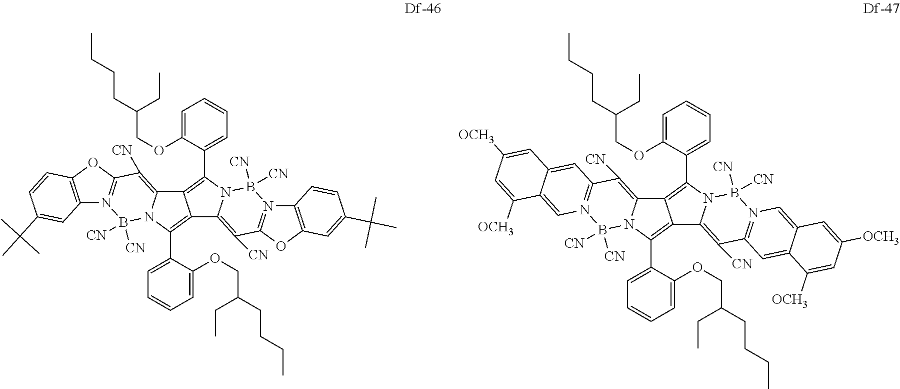

Other squarylium compounds applicable to the present invention include, for example, the exemplified compounds described in paragraphs (0067) to (0069) of JP-A 2018-2773.

The method for synthesizing the squarylium compound according to the present invention is not particularly limited, and may be obtained using, for example, well-known general reactions described in JP-A 5-155144, JP-A 5-239366, JP-A 5-339233, JP-A 2000-345059, JP-A 2002-363434. JP-A 2004.86133, and JP-A 2004-238606.

### [Perylene Compounds]

Next, a representative example of a perylene compound applicable to the present invention will be described.

As an example of the perylene compound applicable to the present invention, a perylene compound described in WO 2018/186462 may be mentioned.

Examples of the perylene compound include a compound having a structure represented by the Formula (15) described in paragraph (0025) of WO 2018/186462 indicated above, and a compound having a structure represented by Formula (39) to Formula (42) described in paragraph (0028).

Specific examples of the compounds include the exemplified compounds 1 to 30 described in paragraphs (0120) to (0124) of WO 2018/16462, the exemplified compounds 80 to 82 described in paragraph (0132), and exemplified compounds 102 to 118 in paragraph (0143) to (0145).

The perylene compound may be synthesized by the method described in paragraphs (135) to (137) of WO 2018/186462 described above.

Further, examples of the other perylene compound include a perylene compound having a structure represented by the following Formula (13).

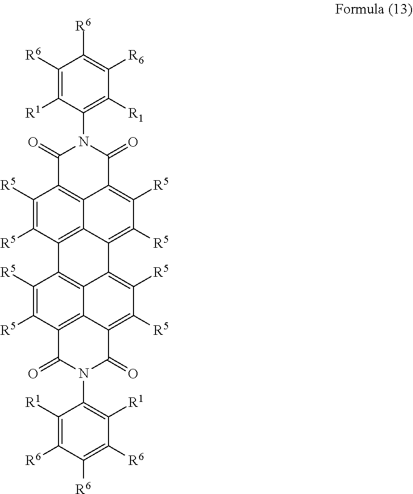

In the above Formula (13), each of a plurality of R¹s independently represents a hydrogen atom or a group having 4 to 30 carbon atoms, and may have an oxygen atom in a carbon chain. Each of a plurality of R⁵s and R⁶s independently represents a hydrogen atom, an alkyl group, an aryl group, a heteroaryl group, an alkenyl group, an alkynyl group, an alkoxy group or an aryloxy group.

However, the aryloxy group represented by R⁵ represents an aryloxy group other than a group having a structure represented by the following Formula (14).

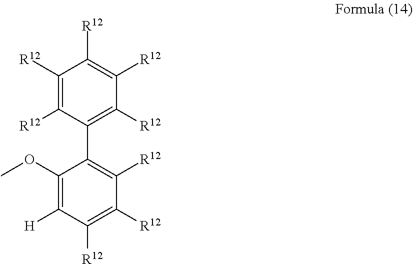

In the above Formula (14), R¹² represents a substituent.

Further, it is preferable that the perylene compound having a structure represented by Formula (13) is a compound having a structure represented by the following Formula (15).

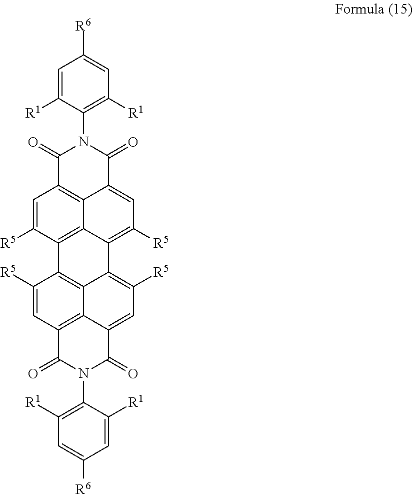

In the above Formula (15), each of a plurality of R¹s independently represents a hydrogen atom or a group having 4 to 30 carbon atoms, and may have an oxygen atom in a carbon chain. Each of a plurality of R⁵s independently represents a hydrogen atom, an alkyl group, an aryl group, a heteroaryl group, an alkenyl group, an alkynyl group, an alkoxy group or an aryloxy group. However, the aryloxy group represented by R⁵ represents an aryloxy group other than the group represented by the above Formula (14). R⁶ each independently represents a hydrogen atom, an alkyl group, an aryl group, a heteroaryl group, an alkenyl group, an alkynyl group, an alkoxy group or an aryloxy group.

Further, it is preferable that the perylene compound represented by the above Formula (15) is a compound having a structure represented by the following Formula (16).

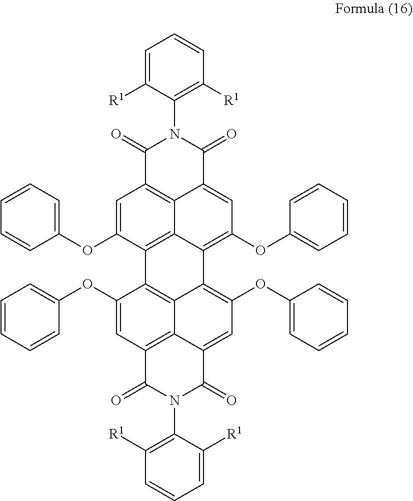

In the above Formula (16), each of a plurality of R¹s independently represents a hydrogen atom or a group having 4 to 30 carbon atoms, and may have an oxygen atom in a carbon chain.

### [Other Luminescent Dyes]

As the luminescent dye according to the present invention, in addition to the squarylium compound and the perylene compound described above, any luminescent dye that satisfies the conditions defined in the present invention may be used without limitation.

Examples of the compound other than the squarylium compound and the perylene compound include a compound containing benzobisthiadiazole having a structure represented by the following Formula (17), a compound containing thiadiazoloquinoxaline having a structure represented by the following Formula (18), a pyrromethene compound having a structure represented by the following Formula (19), and a cyanine compound having a structure represented by the following Formula (20).

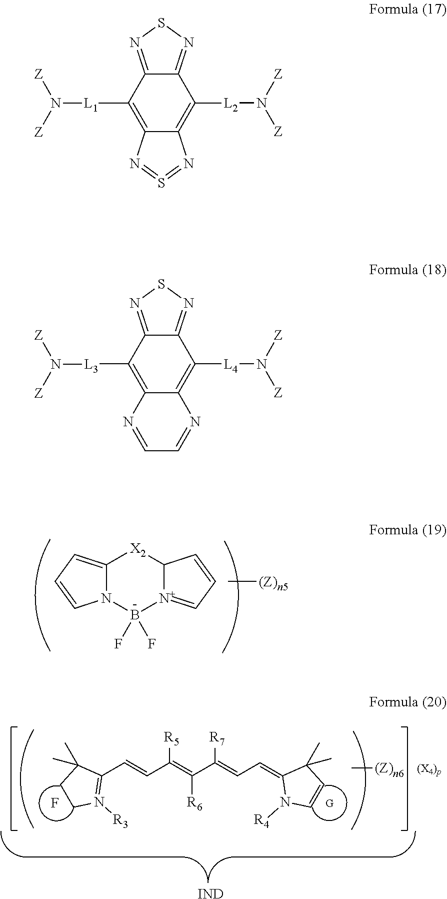

In the above Formula (17) to Formula (20), each of ring F and ring G independently represents an aryl ring.

Each of L₁ to L₄ independently represents an arylene group, a heteroarylene group or a single bond. X₃ represents a carbon atom or a nitrogen atom which may be substituted with an aryl group.

R₃ and R₄ each independently represents an alkyl group which may be substituted with a sulfo group. R₅ and R₇ each independently represents a hydrogen atom or an alkyl group. R₆ represents a hydrogen atom or an aryl group, and when R₅ or R₇ represents an alkyl group, it ay be linked with R₅ or R₇ to form a cycloalkene ring.

When the portion represented by IND is a cationic portion, X₄ represents a counter anion, p represents a number necessary to neutralize the charge, and when the portion represented by IND in the formula is an anionic portion, X₄ represents a counter cation, p represents a number necessary to neutralize the charge, and when the charge of the portion represented by IND in the formula is neutralized in the molecular, p represents 0.

Z represents a group of the following Formula (Z) or a substituent, and at least one of Zs represents the group of Formula (Z).

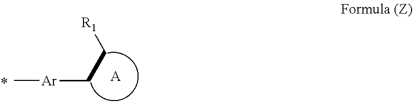

In Formula (Z), ring A represents an aryl ring, a heteroaryl ring or a fused ring thereof. An asterisk (*) represents a binding site with Ar in a luminescent residue. R₁ represents a group in which a dihedral angle between Ar and ring A is 45 degree or more. A thick lite represents a single bond or a double bond.

n5 represents an integer of 1 to 4. n6 represents an integer of 1 to 4.

In addition, in the present invention, as another luminescent dye, a luminescent dye having a structure represented by the following Formula (21) or Formula (22) may be mentioned.

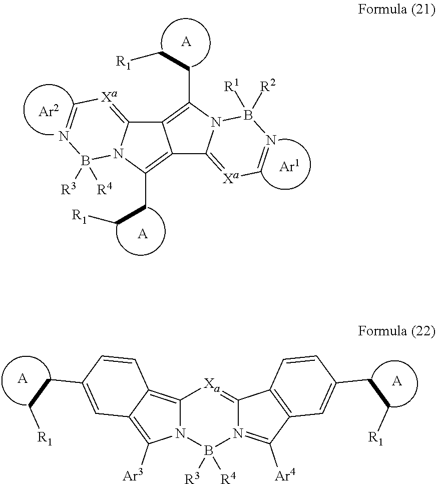

In the above Formula (21) and Formula (22), Ar¹ and Ar² represent an aryl group composed of a 6 membered ring or a heteroaryl group composed of a 5 membered ring or a 6 membered ring, and these groups may contain 2 or more rings.

X^{a} and X^{b} each independently represents a nitrogen atom or CR⁵, and R⁵ represents a hydrogen atom or an electron withdrawing group.

Each of R¹ to R⁴ independently represents a cyano group, a halogen atom, an alkyl group that may be substituted, an alkoxy group that may be substituted, an alkynyl group that may be substituted, an aryl group that may be substituted, or a heteroaryl group that may be substituted.

Ar³ and Ar⁴ represent an aryl group composed of a 6-membered ring or a heteroaryl group composed of a 5-membered ring or a 6-membered ring, and these groups may contain 2 or more rings.

Ring A each independently represents an aryl ring, a heteroaryl ring or a fused ring thereof.

R₁ represents a group having a dihedral angle of 45 degree or more with a pyrrole ring (Formula (21)) or a benzene ring (Formula (22)) to which ring A is bonded, respectively. A thick line represents a single bond or a double bond.

### [Preparation of Wavelength Conversion Film]

The form and manufacturing method of the wavelength conversion film of the present invention are not limited, and they are appropriately determined according to the intended use.

As a form of the wavelength conversion film of the present invention, it may be formed on a support separately from a light-emitting light source (for example, a surface light source) to be described later, and may be superimposed on the surface light source, or may be laminated on the surface light source. Further, it may also serve as an adhesive or a sealing film. As for the thickness, there is no particular limitation as long as the product value (ε×C) of the molar extinction coefficient ε defined in the present invention and the amount of the luminescent dye C (mol) satisfies a condition of 1.0×10⁻³ or more, but is generally preferably within a range of 1 to 1000 μm, and more preferably within a range of 10 to 500 μm.

As a method for manufacturing the wavelength conversion film of the present invention, there may be mentioned a method for temporarily or permanently applying a composition for forming a wavelength conversion film containing a luminescent dye onto a support by a conventionally known thin film forming method, for example, an evaporation method, a sputtering method, a spin coating method, a gravure coating method, or a dip coating method. In the case of manufacturing by a wet method such as a spin coating method, the solvent to be used is not particularly limited, and for example, water, an alcohol, a diol, a ketone, an ester, an ester, an aromatic hydrocarbon (which may contain a halogen atom), or an aliphatic hydrocarbon solvent may be listed.

### [Transparent Matrix Resin]

The wavelength conversion film of the present invention is preferably formed of a luminescent dye and a transparent matrix resin, and further, it is preferable that the transparent matrix resin is at least 1 kind of resin selected from a thermosetting resin, a thermoplastic resin or a photocurable resin.

When the formation of the wavelength conversion film is performed using the composition for forming a wavelength conversion film as described above, a transparent matrix resin is used in order to dissolve or disperse the luminescent dye according to the present invention, but a known transparent resin material may be used as the transparent matrix resin.

In the present invention, from the viewpoint of not affecting the emission wavelength of the luminescent dye according to the present invention, a non-polar resin material is suitably used, and examples thereof include polyolefins such as polystyrene (PS), polyethylene (PE), polypropylene (PP), and polymethylpentene, acrylic resins such as polymethyl methacrylate (PMMA), ethylen-vinyl acetate copolymers (EVA), polyvinyl butyrate (PVB), cellulose esters such as triacetyl cellulose (TAC), and nitrocellulose.

Further, in the composition for forming a wavelength conversion film, various well-known additives such as a colorant, a light stabilizer, an antioxidant, a surfactant, a flame retardant, an inorganic additive, a transparency agent, an ultraviolet absorber, a filler, and light-scattering particles may be appropriately selected and contained in addition to the luminescent dye and the transparent matrix resin if necessary.

### (Light-Scattering Particles)

Among the above, the light-scattering particles are particles having a function of multiple scattering the light that has entered the wavelength conversion film. By adding these, the optical path length of the light entering the wavelength conversion film is extended, and the chance of wavelength conversion inside the wavelength conversion film is increased, so that the wavelength conversion efficiency is improved. Further, the light returned to the inside of the wavelength conversion film is scattered again by the reflection at the wavelength conversion film interface, so that the light extraction efficiency is expected to be improved.

The average particle size of the light-scattering particles is preferably 0.01 μm or more and 10 μm or less, more preferably 0.1 μm or more and 5 μm or less, and still more preferably 0.2 μm or more and 1 μm or less. When the average particle size of the light-scattering particles is less than 0.01 μm, sufficient light-scattering property may not be obtained in the wavelength conversion film, and in order to obtain sufficient light-scattering property, it is necessary to increase the addition amount the light-scattering particles. On the other hand, when the average particle size of the light-scattering particles exceeds 10 μm, the number of light-scattering particles decreases even if the addition amount (mass %) is the same, so that the number of scattering points decreases and a sufficient light-scattering effect may not be obtained.

The shape of the light-scattering particles is not particularly limited, and for example, spherical (true spherical, substantially true spherical, elliptical spherical), polyhedral, rod-shaped (cylindrical, prismatic), flat plate, flaky, and indefinite shape may be cited. When the shape of the light-scattering particles is not spherical, the particle diameter of the light-scattering particles may be a true spherical value having the same volume.

The light-scattering particles are not particularly limited and may be appropriately selected depending on the intended purpose. They may be organic fine particles or inorganic fine particles, but the higher the refractive index, the better the scattering performance of the particles. Therefore, of these, the inorganic fine particles having a high refractive index are preferable.

Examples of the organic fine particles having a high refractive index include polymethylmethacrylate beads, acrylic-styrene copolymer beads, melamine beads, polycarbonate beads, styrene beads, crosslinked polystyrene beads, polyvinyl chloride beads and benzoguanamine-melamine-formaldehyde beads.

Examples of the inorganic fine particles having a high refractive index include inorganic oxide particles composed of at least one oxide of silicon, zirconium, titanium, indium, zinc, antimony, cerium, niobium, or tungsten. Specific examples of the inorganic oxide particles include SiO₂, ZrO₂, TiO₂, BaTIO₃, In₂O₃, ZnO, Sb₂O₃, ITO, CeO₂, Nb₂O₅ and WO₃. Among them, TiO₂, BaTiO₃, ZiO₂, CeO₂ and Nb₂O₅ are preferable, and TiO₂ is the most preferable. Further, among TiO₂, the rutile type is preferable to the anatase type because the catalytic activity is low, the weather resistance of the film is high, and the refractive index is also high.

Further, in order to contain these particles in the wavelength conversion film, those subjected to surface treatment or those not subjected to surface treatment may be selected and used from the viewpoint of improving dispersibility and stability when used as a dispersion liquid.

When surface treatment is performed, specific materials for surface treatment include different inorganic oxides such as silicon oxide and zirconium oxide, metal hydroxides such as aluminum hydroxide, and organic acids such as organosiloxane and stearic acid. These surface treatment materials may be used alone, or a plurality of types may be used in combination. Among them, from the viewpoint of the stability of the dispersion liquid, the surface treatment material is preferably a deferent inorganic oxide, or a metal hydroxide, and more preferably it is a metal hydroxide.

The content of the light-scattering particles with respect to the total solid content mass of the wavelength conversion filter is preferably 0.1% by mass or more and 20% by mass or less, and more preferably 0.2% by mass or more and 5% by mass or less. When the content of the light-scattering particles is less than 0.1% by mass, the light-scattering effect may not be sufficiently obtained. On the other hand, when the content of the light-scattering particles exceeds 20% by mass, the transmittance is lowered because there are too many light-scattering particles.

### (Cut Filter)

Examples of the material of the cut filter include glass and resin. By forming a dielectric multilayer film or a film containing an absorbing dye, a material that excludes light emitted without wavelength conversion may be used. As a cut filter, a commercially available product may be used, or the above film is formed on the wavelength conversion film, or after being produced independently, it may be used in combination with a wavelength conversion film.

### <<Light-Emitting Member>>

The light-emitting member of the present invention is characterized by comprising the wavelength conversion film of the present invention.

FIG. 5 shows an example of the configuration of the light-emitting member of the present invention.

The light-emitting member 11 shown in FIG. 5 shows an example of a light-emitting member that emits light in the near-infrared region. It is a configuration in which is arranged a wavelength conversion film 13 that converts the visible light of the surface light source 12 into near-infrared light IR on a surface light source 12 that emits red light, for example, an organic electroluminescent element (hereinafter, referred to as “organic EL element”) that emits red light.

In the light-emitting member of the present invention, the light source to be applied is, for example, a point light source represented by an LED or a surface light source in which LEDs are integrated, but it may be a surface light source represented by an organic EL element as illustrated in FIG. 5.

The surface light source referred to in the present invention is a light source that emits uniform light from the entire wide surface. On the other hand, as a light source that opposes this, there is a point light source typified by a light emitting diode (Light Emitting Diode: LED) as described above.

In the surface light source according to the present invention, in order to utilize the flexibility of the OLED, it is more preferable to use a flexible resin film as the base material. Examples thereof are: polyesters such as polyethylene terephthalate (PET) and polyethylene naphthalate (PEN), polyethylene, polypropylene, cellophane, cellulose esters and their derivatives such as cellulose diacetate, cellulose triacetate (TAC), cellulose acetate butyrate, cellulose acetate propionate (CAP), cellulose acetate phthalate, and cellulose nitrate, polyvinylidene chloride, polyvinyl alcohol, polyethylene vinyl alcohol, syndiotactic polystyrene, polycarbonate, norbornene resin, polymethyl pentene, polyether ketone, polyimide, polyether sulfone (PES), polyphenylene sulfide, polysulfone, polyether imide, polyether ketone imide, polyamide, fluororesin, Nylon, poly methyl methacrylate, acrylic resin, polyallylate and cycloolefin resins such as ARTON (trade name, made by JSR Co. Ltd.) and APEL (trade name, made by Mitsui Chemicals. Inc.).

### (Organic EL Element)

As the surface light source according to the present invention, it is preferable to apply an organic EL element as described above.

An organic EL element suitable for the present invention as a surface light source includes, for example, a configuration in which an anode and a cathode are provided on a base material, and organic functional layers including a light emitting layer are sandwiched between the anode and the cathode at opposite positions may be mentioned. Further, each functional layer such as a sealing member, a gas barrier layer, and a light extraction layer may be appropriately combined and configured according to the purpose.

Representative element constitutions used for an organic EL element of the present invention are as follows, however, the element constitutions applicable to an organic EL element of the present invention is not limited to these.

(1) Anode/light emitting layer/cathode
(2) Anode/light emitting layer/electron transport layer/cathode
(3) Anode/hole transport layer/light emitting layer/cathode
(4) Anode/hole transport layer/light emitting layer/electron transport layer/cathode
(5) Anode/hole transport layer/light emitting layer/electron transport layer/electron injection layer/cathode
(6) Anode/hole injection layer/hole transport layer/light emitting layer/electron transport layer/cathode
(7) Anode/hole injection layer/hole transport layer/(electron blocking layer/) light emitting layer/(hole blocking layer/) electron transport layer/electron injection layer cathode

The details of each specific constituent layer constituting the organic EL element applicable to the present invention and the manufacturing method thereof are not particularly limited, and known constituent materials and manufacturing methods may be applied. For example, the constituting elements described in JP-A 2013489608, JP-A 2014-120334, JP-A 2015-201508, and WO 2018/51617 may be referred to.

### <<Wavelength Conversion Device>>

The wavelength conversion device provided with the wavelength conversion film of the present invention is preferably a biometric authentication device from the viewpoint of being able to fully exhibit its characteristics. In addition, it can be applied to devices that use near-infrared light, and color filters that may change the color purity by adjusting the emission wavelength and changing the waveform.

The biometric authentication device to which the wavelength conversion film of the present invention may be applied is a device that recognizes an individual by collecting the biological characteristics (movements and a part of the living body) of each individual and measuring and determining the similarity with the characteristic data registered in advance.

Biological information includes veins of palms and fingers, fingerprints, palm shapes, irises, retinas, faces, handwritings, sounds, and odors. Among them, authentication using veins (vein authentication device) has attracted particular attention because of its small size, no risk of theft, and high security.

Vein authentication is an authentication method that utilizes the property of hemoglobin flowing in a vein that absorbs near-infrared light. The light source used in the authentication device is near-infrared light, and it is more preferable that the light source has a maximum emission in the vicinity of 800 nm as described in Opt-Electronics Review, 2017, 25, 263. Vein authentication is performed by irradiating the fingertip with near-infrared rays and collating the obtained image in which the vein portion is shaded with the data registered in advance. Since the amount of data to be extracted is small in the vein authentication method, high-speed processing is possible, and visual confirmation is possible only after irradiating with near-infrared light. Therefore, forgery and theft are less likely to occur as compared with fingerprint authentication, which is a technique for authenticating the same part of a living body. Further, since the vein pattern is information existing inside the living body, it is not easily affected by the outside and does not change semi-permanently, and there are very few maladaptated persons.

A specific configuration of the vein authentication device includes, for example, a light source that emits near-infrared light, a control unit that adjusts the light source, and an imaging unit that detects transmitted light or reflected light from a fingertip to obtain an image, an authentication unit that performs image processing, a storage unit that stores and registers extracted data, and a calculation unit that collates with registered data.

In the current vein authentication, irradiation is generally performed using a plurality of point light sources such as LEDs. However, since the LED is a point light source having a spherical light emitting portion, the brightness becomes uneven when a plurality of LEDs are arranged, and the recognition rate of ti obtained image pickup is lowered. Further, since the individual light amounts of LEDs are not strictly uniform in the first place, a complicated system is required for a control unit that adjusts the irradiation light amount. Therefore, in the biometric authentication device of the present invention, it is preferable to use a surface light source having luminance uniformity as a light source, more specifically, an OLED, and to arrange the wavelength conversion film of the present invention on the light emitting surface side thereof.

The biometric authentication device of the present invention may be mounted on various devices, and one of the features is that it is mounted on a wristband-type electronic device.

The wristband-type electronic device of the present invention is not particularly limited, and examples thereof include a bracelet, a wristwatch, a smart watch, and a wristwatch type smartphone. They are devices capable of transmitting biometric authentication information by wireless communication.

As a preferred embodiment of the wristband type-electronic device of the present invention, the light source is provided in any region on the wristband other than the same plane as the imaging unit when the wristband-type electronic device is worn. By providing the light source in any region on the wristband other than the same plane as the imaging unit at the time of wearing, it is possible to reduce the influence of biological information unnecessary for authentication by the reflected light on the biological surface. Since it is possible to receive biological information in the near-infrared light scattered in the living body, vein imaging is facilitated. The imaging portion of the wrist is not particularly limited, but vein imaging on the outside or inside of the wrist is suitable for authentication.

The position of the light source is not limited as long as it is in any region on the wristband other than on the same plane as the imaging unit when mounted. However, in order to further reduce the influence of biometric information unnecessary for authentication, the angle formed by the center point of the imaging unit, the center point of the light source, and the center point of the wrist cross section is preferably in the range of 30 to 180 degree, more preferably in the range of 45 to 180 degree, and still more preferably in the range of 90 to 180 degree.

The number of light sources is not particularly limited, but it is preferable to install them in the range of 1 to 3 from the viewpoint of power consumption.

The imaging unit is not particularly limited, but it is preferable to use a wide-angle camera because the vein pattern is photographed at a wide angle.

### <<Biometric Device>>

Biometric devices are now an indispensable device for daily health management and medical practice in society. On the other hand, since it is necessary to attach the sensor to the body, compatibility with QOL (quality of life, also referred to as “quality of living”) has become an issue.

Since the light-emitting member of the present invention emits light in the region of the “biological window”, it is suitable for mounting on a biometric measuring apparatus.

The biometric device provided with the light-emitting member of the present invention is not particularly limited, and examples thereof include a pulse oximeter and a pulse wave sensor. With a pulse oximeter, the oxygen concentration in blood may be measured using two wavelengths, near-infrared light and red light.

Since pulse oximeters and pulse wave sensors in medical settings are generally attached to a fingertip, they reduce the QOL of inpatients. Since the pulse oximeter and pulse wave sensor provided with the light-emitting member of the present invention use surface light sources, it enables sensing at thick bones (a wrist and base of a finger), which is difficult with conventional point light sources, and fingertips. It is possible to eliminate the troublesomeness of the fingertip. The reason is not clear, but it is speculated as follows.

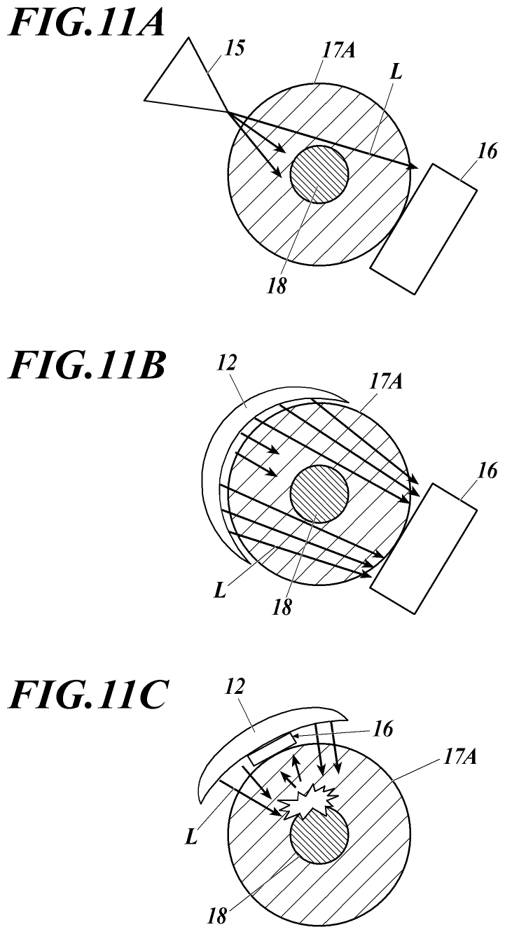

FIG. 11A to FIG. 11C each are a schematic view showing an example of sensing at a fingertip using a point light source and a surface light source as a light-emitting member.

FIG. 11A shows transmission type sensing of a fingertip by a conventional point light source 15, and since a bone 18 is thin at a tip portion 17A of the finger, even the point light source 15 passes through the living body, the light L is transmitted and achieves to a sensor 16.

FIG. 11B shows transmission type sensing of a fingertip using a surface light source 12 of the present invention. The surface light source 12 of the present invention has a large light emitting area and the directivity of the light L is not strong. Therefore, the restriction on the alignment between the surface light source 12 and a sensor 16 is remarkably reduced, and the light L stably passes through the living body and reaches the sensor 16.

FIG. 11C shows reflection type sensing of a fingertip using a surface light source 12 of the present invention. The surface light source 12 of the present invention has a large light emitting area and the directivity of the light L is not strong. Therefore, the scattering of the light L in the living body is promoted, and the light L is stably reflected in the living body and reaches the sensor 16.

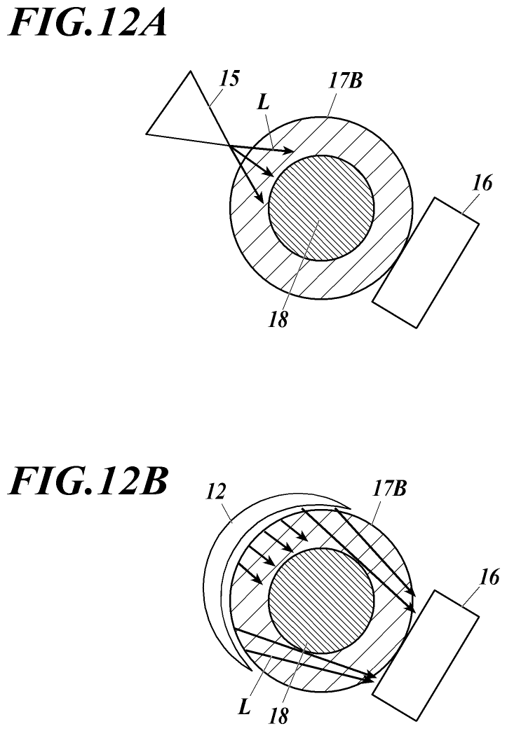

FIG. 12A and FIG. 12B each are a schematic view showing an example of sensing at a base of a finger using a point light source and a surface light source as a light-emitting member. FIG. 12A shows sensing using a conventional point light source 15. As shown in FIG. 12A, at a base of a finger 17B, a conventional point light source 15 has a small light emitting area and strong directivity of light L. Therefore, the light L is often blocked by a bore 18, and the alignment between the point light source 15 and a sensor 16 is limited to a very narrow range, which makes stable sensing difficult.

On the other hand, in the case of sensing at a base of a finger using a surface light source 12 of the present invention, as shown in FIG. 12B, the light emitting area is large and the directivity of the light L is not strong. Therefore, the restriction on the alignment between the surface light source 12 and a sensor 16 is remarkably reduced, and the light L stably passes through the living body and reaches the sensor 16.

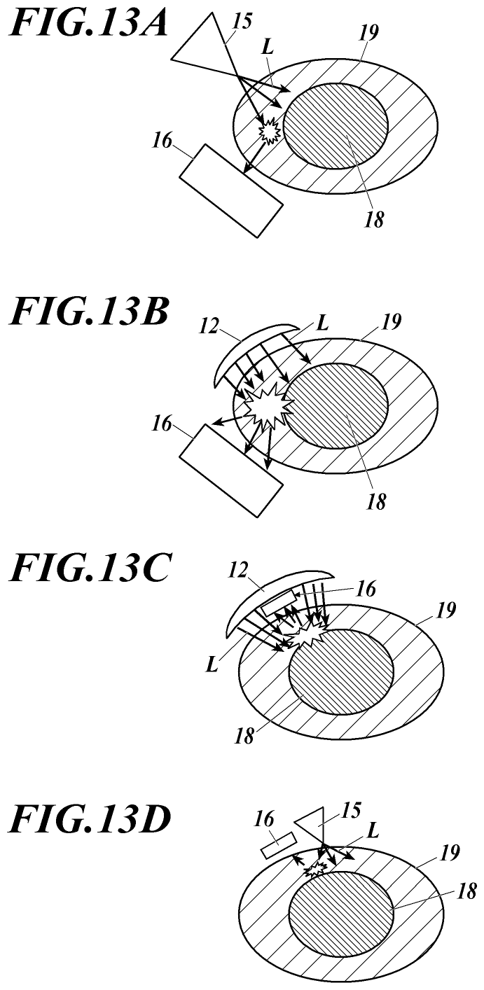

FIG. 13A to FIG. 13D each are a schematic view showing an example of sensing on a wrist using a point light source and a surface light source.

A conventional point light source 15 is used for V-shaped sensing in FIG. 13A and reflection type wrist sensing in FIG. 13D. Since the directivity of the light L is strong in these sensing, there is a problem that the light L is not sufficiently scattered in the living body and a sufficient amount of light for sensing may not be received. On the other hand, in the V-shaped sensing on a wrist shown in FIG. 13B, since the surface light source 12 which is the light-emitting member of the present invention is used, the light L enters the living body over a wide area, and light scattering in the living body becomes active. It is possible to obtain biological information with the amount of light required for sensing. Similarly, the same effect as that of FIG. 13B may be obtained in the reflection type wrist sensing shown in FIG. 13C.

### EXAMPLES

Hereinafter, the present invention will be specifically described with reference to Examples, but the present invention is not limited thereto. Unless otherwise specified, “%” and “part” mean “mass %” and “part by mass”, respectively.

### Example 1

### <<Preparation of Wavelength Conversion Film>>

### [Preparation of Wavelength Conversion Film 1]

A wavelength conversion film 1 was prepared according to the following method.

A mixture composed of polystyrene (manufactured by ACROS ORGANICS Co., Ltd., weight average molecular weight Mw=260000) as a matrix material and a luminescent dye 1 (structure is separately described) was sufficiently dissolved in toluene as a solvent by heating and stirring at 80° C., thereby preparing a luminescent dye solution 1.

Then, the obtained luminescent dye solution 1 was applied onto a polyethylene terephthalate film using an applicator, dried at room temperature for 30 minutes, and then further heated and dried at 80° C. for 30 minutes to prepare a wavelength conversion film 1.

### [Preparation of Wavelength Conversion Films 2 to 43]

In the preparation of the wavelength conversion film 1 described above, the wavelength conversion films 2 to 43 were prepared in the same manner except that the type of the luminescent dye and the amount of the luminescent dye (C) corresponding to a thickness of the film per 1 cm² was changed.

Details of the luminescent dyes used in the preparation of the wavelength conversion films 1 to 43 described above are as follows.

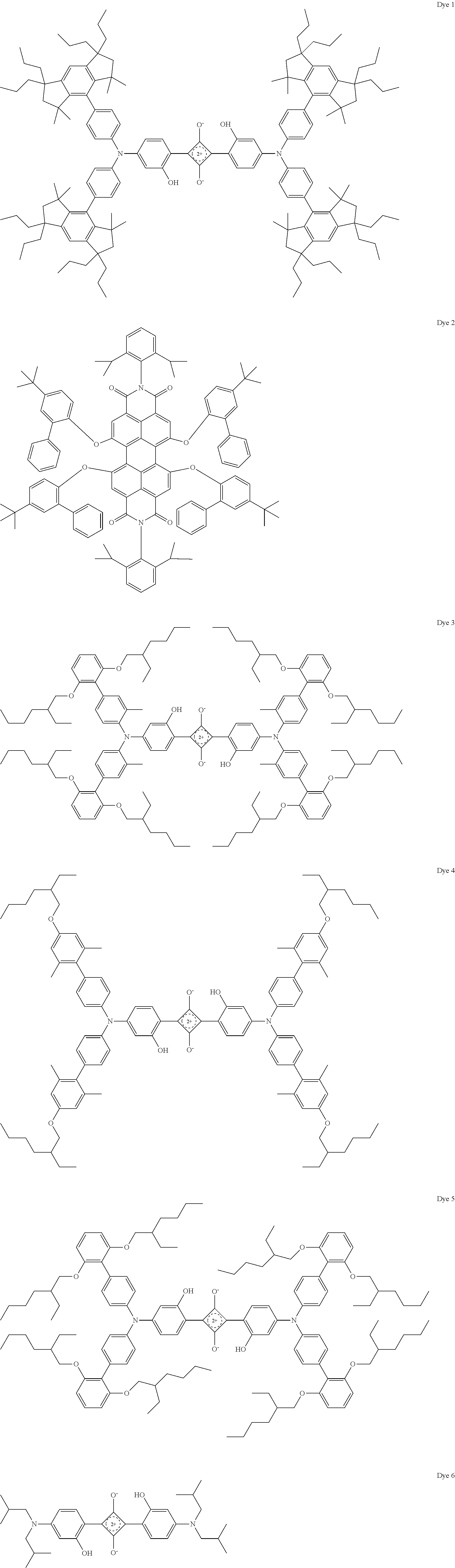

### <<Measurement of Characteristic Values of Luminescent Dye and Wavelength Conversion Film>>

### [Measurement of Molar Extinction Coefficient ε of Luminescent Dye]

First, the absorption spectrum and the emission spectrum of the luminescent dyes 1 to 6 were measured according to the following method.

The value of the molar extinction coefficient was measured using a spectrophotometer U-3300 (manufactured by Hitachi High-Tech Science Corporation). A solution in which a luminescent dye was dissolved in toluene at a concentration of 1×10⁻⁶ to 1×10⁻⁴ mol/L was prepared, and the value of the molar extinction coefficient was calculated from the dye concentration. The measured concentration of each dye was appropriately adjusted so that the maximum absorbance was 1.0 or less.

Subsequently, in order to obtain the intersection of the emission spectrum and the absorption spectrum, a wavelength conversion film was prepared in which the dye concentration and film thickness were adjusted so that the amount of dye C×the molar extinction coefficient ε was in the range of 1.0×10⁻⁴ or C×the molar extinctiless. Then, an emission spectrum and an absorption spectrum were measured using a fluorometer F-7000 (manufactured by Hitachi High-Tech Science Corporation) and a spectrophotometer U-3300 (manufactured by Hitachi High-Tech Science Corporation), respectively.

Next, the peak intensities of the maximum emission wavelength of the obtained absorption spectrum and the emission spectrum were normalized at the same intensity as shown in FIG. 1, and then the intersection point P of the two spectrums was obtained. The molar extinction coefficient at the same absorption intensity in the normalized absorption spectrum of the solution at the intersection point P was defined as the molar extinction coefficient e of the luminescent dye.

### [Calculation of ε×C Value]

The product value (ε×C) of the above measured molar extinction coefficient ε and the amount of luminescent dye C (mol) according to a film thickness per 1 cm² of each wavelength conversion film was calculated.

The amount of luminescent dye C according to a film thickness per 1 cm² of the wavelength conversion film was determined from the luminescent dye concentration and the film thickness.

The thickness of each wavelength conversion film was measured at 10 points while shifting the position of each wavelength conversion film using a film thickness meter Digimicro MH-15M (manufactured by Nikon Corporation), and the film thickness was averaged.

### [Measurement of Absolute Quantum Yield of Wavelength Conversion Film]

Absolute quantum yields of the wavelength conversion films 1 to 43 were measured using a Quantaurus-QY absolute PL-quantum yield measuring apparatus: C11347-01 (manufactured by Hamamatsu Photonics K.K.). Further, relative quantum yields of the other wavelength conversion films were obtained when the absolute quantum yield obtained from the wavelength conversion film 34 (Comparative Example) was set to 1.00.

### [Measurement of Wavelength Change Width Δλ of Wavelength Conversion Film]

Next, according to the methods described in FIG. 3A, FIG. 3B and FIG. 4, the wavelength difference value Δλ at an intensity of 20% on the respective short-wave sides of the emission spectrum A and the emission spectrum B were determined from the emission spectrum A detected by the configuration in which the excitation light source and the detection unit were arranged on the same plane side with respect to the wavelength conversion film and the emission spectrum B detected by the configuration in which the excitation light source and the detection unit were arranged at positions facing each other across the wavelength conversion film.

First, the reflection type emission spectrum A was measured using a fluorometer F-7000 (manufactured by Hitachi High-Tech Science Corporation) as a measuring apparatus, and using the excitation light source 2 mounted on the apparatus in the configuration described in FIG. 3A.

Next, the transmission type emission spectrum B was measured by the detection unit 3 using a fluorometer F-7000 (manufactured by Hitachi High-Tech Science) as a measuring apparatus, using LEDs as the excitation light source 2 in the configuration described in FIG. 3B, and irradiating from the back surface of the respective wavelength conversion films.

Then, the obtained reflection type emission spectrum A, and the transmission type the emission spectrum B were subjected to aligning the maximum emission peak value as shown in FIG. 2 to 100%, to determine the wavelength difference value Δλ of both spectrums at an intensity of 20% on the short-wave side.

The obtained results by the above are shown in Table XIII and Table XIV.

TABLE XIII

| | | | | Wavelength change | |
|---|---|---|---|---|---|
| Wavelength | | Dye amount C × | Relative | width Δ λ at | |
| conversion | Luminescent | Molar extinction | quantum | a luminescence | |
| film number | dye type | coefficient ε | yield | intensity of 20% (nm) | Remarks |
| 1 | Dye 1 | 1.50 × 10⁻³ | 1.30 | 21 | Present Invention |
| 2 | Dye 1 | 2.37 × 10⁻³ | 1.26 | 32 | Present Invention |
| 3 | Dye 1 | 1.02 × 10⁻³ | 1.22 | 10 | Present Invention |
| 4 | Dye 1 | 2.00 × 10⁻³ | 1.17 | 25 | Present Invention |
| 5 | Dye 1 | 2.99 × 10⁻³ | 1.09 | 29 | Present Invention |
| 6 | Dye 1 | 4.54 × 10⁻³ | 1.13 | 39 | Present Invention |
| 7 | Dye 1 | 5.74 × 10⁻³ | 1.13 | 43 | Present Invention |
| 8 | Dye 1 | 2.85 × 10⁻³ | 0.87 | 27 | Present Invention |
| 9 | Dye 1 | 4.38 × 10⁻³ | 0.91 | 34 | Present Invention |
| 10 | Dye 1 | 3.80 × 10⁻³ | 0.83 | 33 | Present Invention |
| 11 | Dye 1 | 6.26 × 10⁻³ | 0.83 | 42 | Present Invention |
| 12 | Dye 1 | 4.48 × 10⁻³ | 0.65 | 34 | Present Invention |
| 13 | Dye 1 | 7.47 × 10⁻³ | 0.70 | 43 | Present Invention |
| 14 | Dye 1 | 3.52 × 10⁻⁴ | 1.57 | 7 | Present Invention |
| 15 | Dye 1 | 6.26 × 10⁻⁴ | 1.83 | 6 | Present Invention |
| 16 | Dye 2 | 1.01 × 10⁻³ | 4.04 | 11 | Present Invention |
| 17 | Dye 2 | 1.14 × 10⁻³ | 4.00 | 10 | Present Invention |
| 18 | Dye 2 | 2.03 × 10⁻³ | 3.96 | 12 | Present Invention |
| 19 | Dye 2 | 3.12 × 10⁻³ | 3.74 | 12 | Present Invention |
| 20 | Dye 2 | 6.17 × 10⁻³ | 3.70 | 22 | Present Invention |
| 21 | Dye 2 | 5.44 × 10⁻³ | 3.43 | 17 | Present Invention |
| 22 | Dye 2 | 9.49 × 10⁻³ | 3.30 | 25 | Present Invention |
| 23 | Dye 2 | 1.19 × 10⁻⁴ | 4.00 | 0 | Comparative Example |
| 24 | Dye 2 | 2.05 × 10⁻⁴ | 4.09 | 1 | Comparative Example |
| 25 | Dye 2 | 5.85 × 10⁻⁴ | 4.04 | 8 | Comparative Example |

TABLE XIV

| | | | | Wavelength change | |
|---|---|---|---|---|---|
| Wavelength | | Dye amount C × | Relative | width Δ λ at | |
| conversion | Luminescent | Molar extinction | quantum | a luminescence | |
| film number | dye type | coefficient ε | yield | intensity of 20% (nm) | Remarks |
| 26 | Dye 3 | 1.08 × 10⁻³ | 1.91 | 10 | Present Invention |
| 27 | Dye 3 | 3.19 × 10⁻³ | 1.65 | 15 | Present Invention |
| 28 | Dye 3 | 6.38 × 10⁻³ | 1.70 | 26 | Present Invention |
| 29 | Dye 3 | 1.00 × 10⁻² | 1.70 | 34 | Present Invention |
| 30 | Dye 3 | 9.57 × 10⁻³ | 1.35 | 28 | Present Invention |
| 31 | Dye 3 | 1.58 × 10⁻² | 1.17 | 37 | Present Invention |
| 32 | Dye 3 | 5.88 × 10⁻⁴ | 2.04 | 6 | Comparative Invention |
| 33 | Dye 4 | 1.03 × 10⁻³ | 0.78 | 18 | Present Invention |
| 34 | Dye 4 | 5.53 × 10⁻⁴ | 1.00 | 7 | Comparative Invention |
| 35 | Dye 5 | 1.24 × 10⁻³ | 0.91 | 11 | Present Invention |
| 36 | Dye 5 | 2.11 × 10⁻³ | 0.96 | 19 | Present Invention |
| 37 | Dye 5 | 2.62 × 10⁻³ | 0.65 | 18 | Present Invention |
| 38 | Dye 5 | 4.23 × 10⁻³ | 0.65 | 22 | Present Invention |
| 39 | Dye 5 | 5.06 × 10⁻⁴ | 1.22 | 0 | Comparative Invention |
| 40 | Dye 5 | 9.00 × 10⁻⁴ | 1.09 | 5 | Comparative Invention |
| 41 | Dye 6 | 3.28 × 10⁻³ | 0.70 | 14 | Present Invention |
| 42 | Dye 6 | 1.58 × 10⁻² | 0.74 | 24 | Present Invention |
| 43 | Dye 6 | 7.16 × 10⁻⁴ | 1.74 | 4 | Comparative Example |

As is apparent from the results described in Table XIII and Table XIV, the wavelength conversion film composed of the constitution defined in the present invention was proved to exhibit the following properties. By controlling the molar extinction coefficient of the luminescent dye, the concentration of the luminescent dye, and the product value (ε×C) of the molar extinction coefficient ε determined by the film and the amount of the luminescent dye C (mol), it was found that the wavelength difference value Δλ at an intensity of 20% on the short-wave side of each of the emission spectrums A and B can be shifted to the long wavelength side with a desired wavelength change width, and all of the examples of the present invention have an absolute quantum yield of 10% or more, so that it is also possible to achieve high luminescence. Furthermore, wavelength conversion film numbers 1 to 13, 26 to 31, 33, and 35 to 38 have maximum emission wavelengths exceeding 750 nm, and wavelength conversion film numbers 2, 6 to 13, and 35 to 38 have maximum emission wavelengths exceeding 800 nm.

Further, FIG. 2 shows the results of the emission spectrum of the wavelength conversion film containing Dye 1 in the transmission type.

As is apparent from FIG. 2, the emission wavelength peak becomes longer as the value of the dye amount C×the molar extinction coefficient ε increases. Especially in the case of a large change width, the emission wavelength peak is longer than 50 nm.

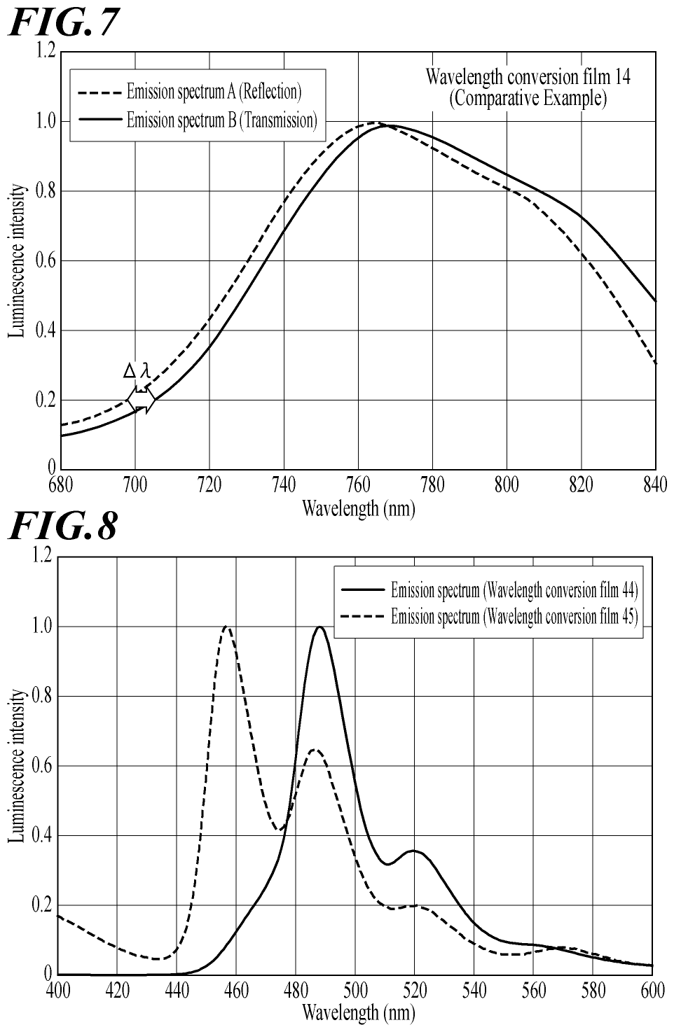

In addition, FIG. 4 shows the results of emission spectrums of the wavelength conversion film 13 (the present invention), and FIG. 7 shows the emission spectrums of the wavelength conversion film 14 (comparative example) measured by the reflection type and the transmission type, respectively.

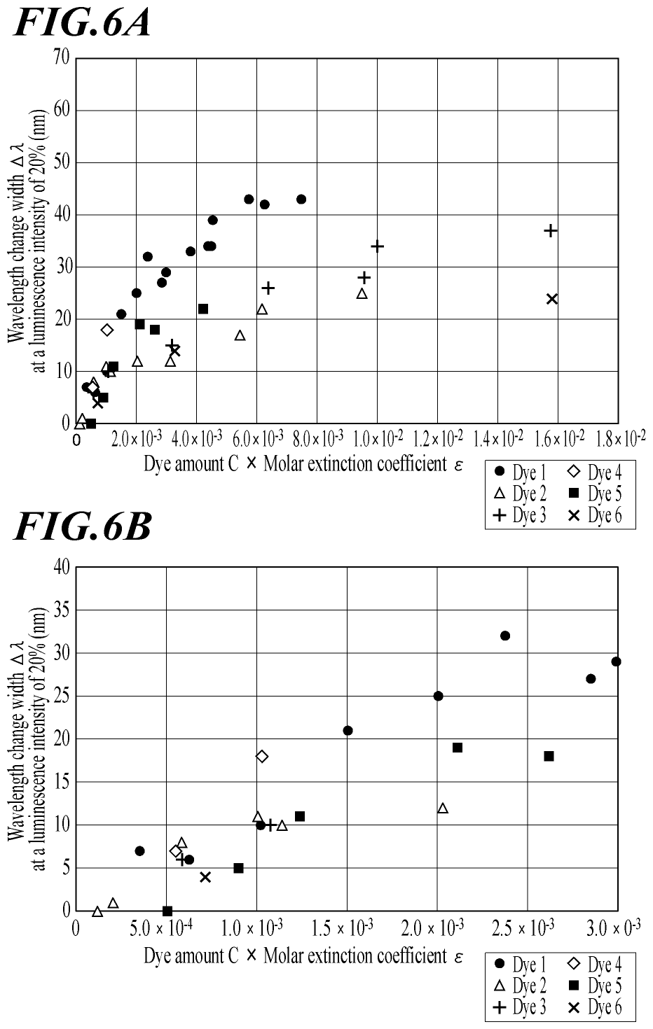

From the comparison of the emission spectrums shown in FIG. 4 and FIG. 7, it can be seen that in the wavelength conversion film 13 in which the value of the dye amount C×the molar extinction coefficient e is within the range of the present invention, the self-absorption phenomenon is apt to occur and the value of the wavelength change width Δλ is large. On the other hand, in the wavelength conversion film 14 in which the value of the dye amount C×the molar extinction coefficient ε is out of the range, as shown in FIG. 7, there is almost no difference between the light emission spectrums of the reflection type and the transmission type, and it is obvious that the self-absorption phenomenon hardly occurs. In addition, FIG. 6A and FIG. 6B are scatter diagrams showing the relation between the dye amounts C×molar extinction coefficients e of the luminescent dyes (Dye 1 to Dye 6) used in the above examples and the wavelength change width Δλ at 20% luminescence intensity.

FIG. 6A shows the total range of the dye amount C×the molar extinction coefficient ε applied in the examples, and FIG. 6B shows an enlarged view in the vicinity of thresholds.

As is clear from the scatter diagrams shown in FIG. 6A and FIG. 6B, since the self-absorption phenomena of the luminescent dyes according to the present invention strongly appear as the amount of dye C×the molar extinction coefficient ε increases, the wavelength change width Δλ shown on the vertical axis of the luminescent dyes according to the present invention proportionally increase.

### Example 2

### <<Preparation of Wavelength Conversion Film>>

### [Preparation of Wavelength Conversion Films 44 and 45]

In the preparation of the wavelength conversion film 1 described in Example 1, the luminescent dye to be used was changed from Dye 1 to Dye 7, and the dye amount C corresponding to the film thickness per 1 cm² was changed to prepare a wavelength conversion film 44 (the present invention) and a wavelength conversion film 45 (comparative example).

The structure of Dye 7 used is shown below.

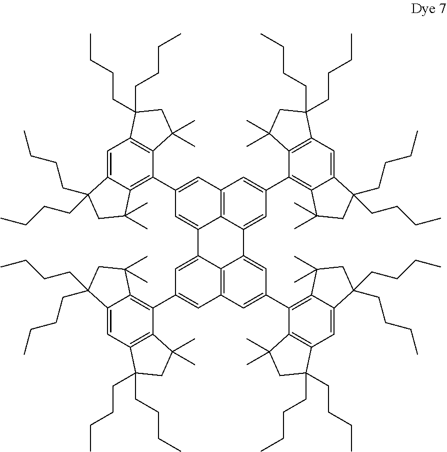 [SMILES: CCCCC1(CCCC)CC(C)(C)c2c1cc1c(c2-c2cc3cc(-c4c5c(cc6c4C(C)(C)CC6(CCCC)CCCC)C(CCCC)(CCCC)CC5(C)C)cc4c5cc(-c6c7c(cc8c6C(C)(C)CC8(CCCC)CCCC)C(CCCC)(CCCC)CC7(C)C)cc6cc(-c7c8c(cc9c7C(C)(C)CC9(CCCC)CCCC)C(CCCC)(CCCC)CC8(C)C)cc(c(c2)c34)c65)C(C)(C)CC1(CCCC)CCCC]

With respect to the wavelength conversion films 44 and 45 prepared above, the dye amount C×the molar extinction coefficient ε was calculated according to the “Measurement of molar extinction coefficient ε of luminescent dye” and the “Calculation of ε×C value” method described in Example 1.

The results obtained from the above am shown in Table XV.

TABLE XV

| Wavelength | | Dye amount C × | |
|---|---|---|---|
| conversion film | Luminescent | Molar extinction | |
| number | dye type | coefficient ε | Remarks |
| 44 | Dye 7 | 7.20 × 10⁻³ | Present Invention |
| 45 | Dye 7 | 1.30 × 10⁻⁵ | Comparative Example |

Next, the wavelength conversion films 44 and 45 thus produced were measured using a fluorometer F-7000 (manufactured by Hitachi High-Tech Science Corporation) as a measuring apparatus. LEDs were used as the excitation light source 2 in the configuration described in FIG. 3B, and the reflection type emission spectrums were measured.

The results of the emission spectrum and the absorption spectrum obtained by the above measurement are shown in FIG. 8. Wavelength conversion film 44 of the present invention has a narrow emission spectral width with respect to the wavelength conversion film 45 of a comparative example, and it was confirmed that it is excellent in color purity of the emission color.

### Example 3

### <<Preparation of Wavelength Conversion Film>>

### [Preparation of Wavelength Conversion Films 46 to 57]

In the preparation of the wavelength conversion film 1 described in Example 1, wavelength conversion films 46 to 51 and 55 to 57 were prepared in the same manner except that the type of luminescent dye and the amount of luminescent dye C according to a film thickness per 1 cm² of the film were changed. In addition, the wavelength conversion films 52 to 54 were produced by mixing Dye 9 and Dye 10, but the light emission of Dye 9 is absorbed by Dye 10 because of the relationship between the absorption and the emission wavelength of both the dyes, so that the wavelength conversion film is one in which the light emission of Dye 10 is observed.

The structures of dyes 8 to 11 used are shown below.

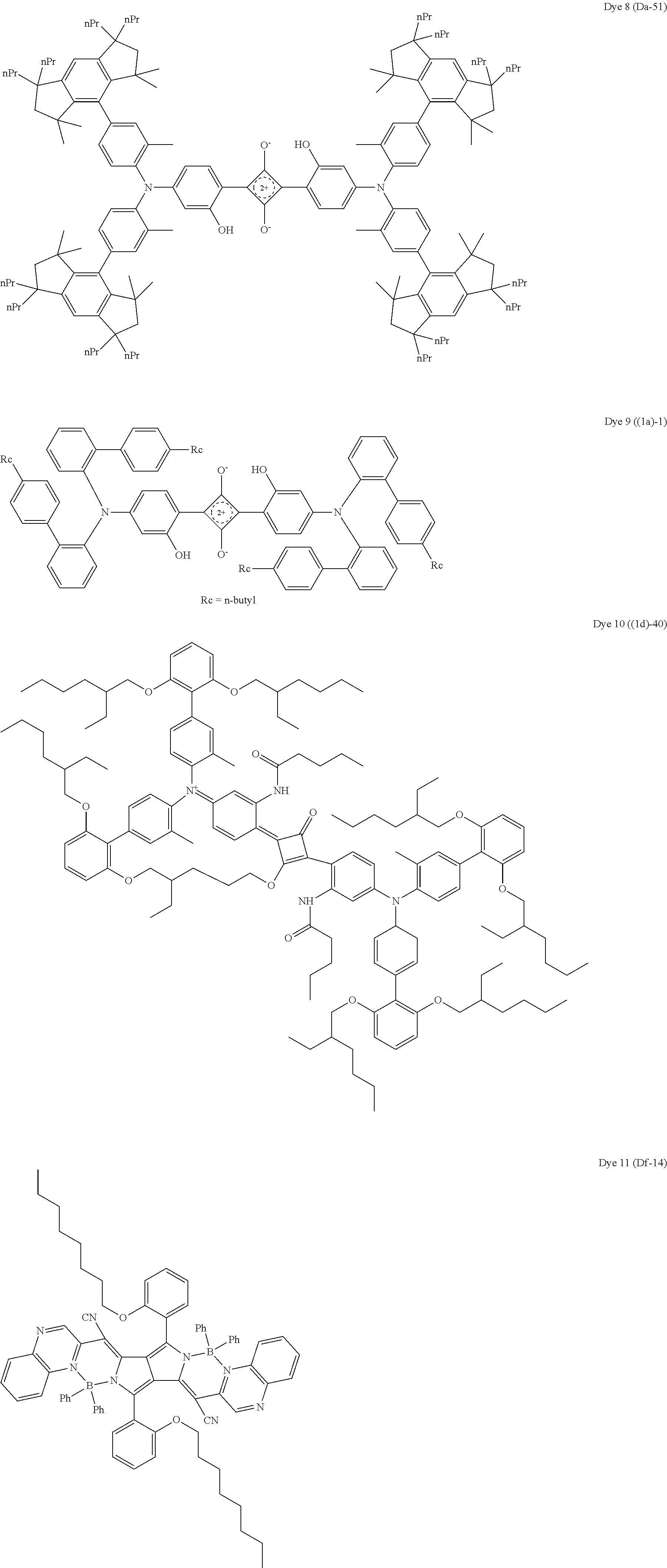

Similar to the method described in Example 1, the molar extinction coefficient ε, and ε×C value of the luminescent dye of the produced wavelength conversion film were calculated, further, the relative quantum yield, and the wavelength change width Δλ were measured. The results obtained are shown in Table XVI below.

TABLE XVI

| | | | | Wavelength change | |
|---|---|---|---|---|---|
| Wavelength | | Dye amount C × | Relative | width Δ λ at | |
| conversion | Luminescent | Molar extinction | quantum | a luminescence | |
| film number | dye type | coefficient ε | yield | intensity of 20% (nm) | Remarks |
| 46 | Dye 8 | 4.42 × 10⁻³ | 1.44 | 40 | Present Invention |
| 47 | Dye 8 | 1.58 × 10⁻³ | 1.79 | 23 | Present Invention |
| 48 | Dye 8 | 4.51 × 10⁻⁴ | 2.03 | 6 | Comparative Example |
| 49 | Dye 9 | 5.66 × 10⁻³ | 1.52 | 43 | Present Invention |
| 50 | Dye 9 | 1.78 × 10⁻³ | 1.65 | 25 | Present Invention |
| 51 | Dye 9 | 4.31 × 10⁻⁴ | 2.11 | 5 | Comparative Example |
| 52 | Dye 9 | 6.75 × 10⁻³ | 0.73 | 35 | Present Invention |
| | Dye 10 | | | | |
| 53 | Dye 9 | 1.84 × 10⁻³ | 1.21 | 17 | Present Invention |
| | Dye 10 | | | | |
| 54 | Dye 9 | 3.73 × 10⁻⁴ | 1.64 | 5 | Comparative Example |
| | Dye 10 | | | | |
| 55 | Dye 11 | 5.61 × 10⁻³ | 0.88 | 22 | Present Invention |
| 56 | Dye 11 | 2.01 × 10⁻³ | 1.03 | 14 | Present Invention |
| 57 | Dye 11 | 5.55 × 10⁻⁴ | 1.43 | 8 | Comparative Example |

As is apparent from the results described in Table XVI, the wavelength conversion film composed of the constitution defined in the present invention was proved to exhibit the following properties. By controlling the molar extinction coefficient of the luminescent dye, the concentration of the luminescent dye, and the product value (ε×C) of the molar extinction coefficient ε determined by the film and the amount of the luminescent dye C (mol), it was found that wavelength difference value Δλ at an intensity of 20% on the short-side of each of the emission spectrum A and the emission spectrum B can be shifted to the long wavelength side with a desired wavelength change width, and all of the examples of the present invention have an absolute quantum yield of 10% or more, so that it is also possible to achieve high luminescence. Furthermore, wavelength conversion film numbers 46 to 47, 49 to 50, 52 to 53, and 55 to 56 have maximum emission wavelengths exceeding 750 nm, and wavelength conversion film numbers 52 to 53 and 55 to 56 have maximum emission wavelengths exceeding 800 nm.

### Example 4

### <<Preparation of Organic EL Element A>>

According to the following procedure, anode/hole injection layer/hole transport layer/light emitting layer/electron transport layer/electron injection layer/cathode were laminated in this order on the substrate and sealed. Thus a bottom emission type organic EL element A was produced.

### (Formation of Anode)

First, on the entire surface of the side of the polyethylene naphthalate film (manufactured by Teijin DuPont Co., Ltd., hereinafter abbreviated as PEN) that forms an anode, an inorganic gas barrier layer made of SiOₓ was formed so as to have a thickness of 500 nm by using an atmospheric pressure plasma discharge processing apparatus having the configuration described in JP-A-2004-68143. As a result, a flexible base material (gas barrier film) having a gas barrier property with an oxygen permeability of 0.001 mL/m²·24 h·atm) or less and a water vapor permeability of 0.001 g/(m²·24 h) or less was prepared.

Next, ITO (In₂O₃:SnO₂=90:10 mass % ratio) was formed by a sputtering method so that the thickness was 150 nm, and then patterning was performed to form an anode. The pattern had an area of a light emitting region of 10 mm×40 mm. Subsequently, after ultrasonic cleaning with isopropyl alcohol, the anode was dried with dry nitrogen gas, and UV ozone cleaning was performed for 5 minutes.

### (Formation of Organic Functional Layer Group)

The anode produced by the above method was dried in a glove box having a dew point of −80° C. or less and an oxygen concentration of 1 ppm or less, and then transferred into a vacuum vapor deposition apparatus. The crucible (made of molybdenum or tungsten, which is a material for resistance heating) in the vacuum vapor deposition apparatus was filled with the constituent materials of each of the organic functional layers described below in an amount required for producing an organic EL element.

### <Formation of Hole Injection Layer>

The vacuum vapor deposition apparatus was depressurized to 1×10⁻⁴ Pa, and the compound HIL-1 (MTDATA) was vapor-deposited at a vapor deposition rate of 0.1 nm/sec, and a hole injection layer having a thickness of 15 nm (hereinafter referred to as HIL) was formed.

### <Formation of Hole Transport Layer>

Next, the following compound HTL-1 (α-NPD) was deposited on the HIL to form a hole transport layer (HTL) having a thickness of 30 nm.

### <Formation of Red Phosphorescent Light Emitting Layer>

Subsequently, the heating boats each containing the light emitting host compound H-1 and the dopant compound DP-1 were independently energized, and the vapor deposition rates of H-1 and DP-1 were adjusted to be 100:6. Then, a red phosphorescent light emitting layer (EML) having a thickness of 20 nm was formed.

### <Formation of Electron Transport Layer>

Next, the heating boat containing tris(8-hydroxyquinolinate)aluminum (Alq3) was energized and heated to be vapor-deposited at a vapor deposition rate of 0.1 nm/sec, and an electron transport layer (ETL) having a thickness of 25 nm was formed on the EML.

### <Formation of Electron Injection Layer>

Next, LiF was deposited at a vapor deposition rate of 0.1 nm/sec to form an electron injection layer (EIL) having a thickness of 1 nm.

### (Formation of Cathode)

Subsequently, aluminum was deposited to a thickness of 70 nm to form a cathode.

### (Formation of Sealing Structure)

Next, a sealing base material was adhered onto the cathode of the above-mentioned laminate formed from the anode to the cathode using a commercially available roll laminating device. The sealing base material was prepared as follows. A flexible aluminum foil (manufactured by Toyo Aluminum K.K. Co., Ltd.) with a thickness of 30 μm was provided with an adhesive layer having a thickness of 1.5 μm using a two-component reaction type urethane adhesive for dry lamination. Then, a 12 μm polyethylene terephthalate (PET) film was laminated thereon to obtain the sealing base material.

A thermosetting adhesive was uniformly applied along the adhesive surface (glossy surface) of the aluminum foil of the sealing base material using a dispenser to form an adhesive layer having a thickness of 20 μm. This was dried under a reduced pressure of 100 Pa or less for 12 hours. As the thermosetting adhesive, a mixture of the following components (A) to (C) was used.

(A) Bisphenol A diglycidyl ether (DGEBA)

(B) Dicyandiamide (DICY)

(C) Epoxy Adduct-Based Curing Accelerator

Next, the sealing base material was transferred to a nitrogen atmosphere having a dew point temperature of −80° C. or less and an oxygen concentration of 0.8 ppm, dried for 12 hours or longer, and adjusted so that the water content of the sealing adhesive was 100 ppm or less.

Finally, the sealing base material was closely adhered and arranged with respect to the laminate, and was tightly sealed using a pressure-bonding roll under the conditions of a temperature of 100° C. with a pressure of 0.5 MPa, and an apparatus speed of 0.3 m/min. Then, the adhesive was cured by subjecting it to heat treatment at 110° C. for 30 minutes to obtain an organic EL element A.

The details of each constituent material used for producing the organic EL element A are as follows.

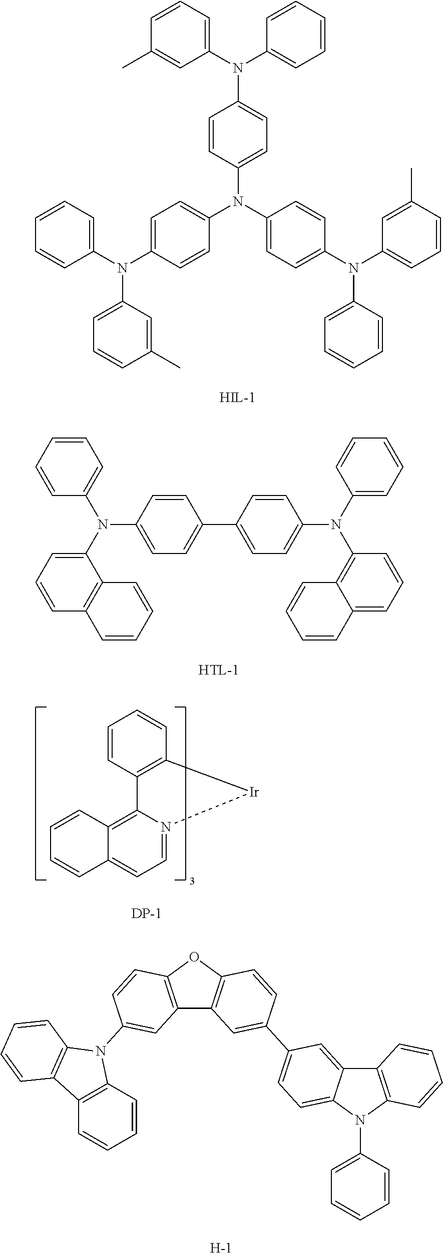

### <<Preparation of Light-Emitting Member>>

### (Preparation of Light-Emitting Member 1-1)

The member having only the surface light source (organic EL element A) produced above was designated as a light-emitting member 1-1.

### (Preparation of Light-Mitting Member 1-2)

The light emitting surface of the organic EL element A, which is the surface light source produced above, and the produced wavelength conversion film 53 were brought into close contact with each other to produce a light-emitting member 1-2 having the configuration shown in FIG. 5.

### Example 5

A wristband-type electronic device equipped with a biometric authentication device was produced and evaluated according to the following method.

(Preparation of Wristband-Type Electronic Device Equipped with Biometric Authentication Device)

A wristband-type electronic device 20 equipped with a biometric authentication function by imaging the wrist vein was produced. As shown in FIG. 10, this electronic device 20 contains an imaging unit 21 composed of a camera and a wide-angle lens placed on the outside of the wrist, and two light-emitting members 1-2 prepared in Example 4 provided on a wristband other than on the same plane as the imaging unit when attached.

(Imaging with Wristband-Type Electronic Device Equipped with Biometric Authentication Device)

When the wristband-type electronic device 20 shown in FIG. 10 was attached on a wrist, it was possible to take an image of a clear vein pattern peculiar to the individual.

### Example 6

A biometric authentication device using vein imaging of the fingertip was prepared and evaluated according to the following method.

### (Preparation of Biometric Authentication Device Using Vein Imaging of Fingertip)

As shown in FIG. 11B, the surface light source 12 and the sensor 16 were arranged so as to face each other with the finger 17A sandwiched between them, and a device capable of biometric authentication by vein imaging of the fingertip was produced.

(Imaging with Biometric Authentication Device Using Vein Imaging of Fingertip)

By performing vein imaging of a fingertip using the biometric authentication device shown in FIG. 11B, it was possible to take an image of the vein pattern peculiar to the individual.

### Example 7

A pulse oximeter was prepared and evaluated according to the following method.

### (Preparation of Light-Emitting Member 1-3)

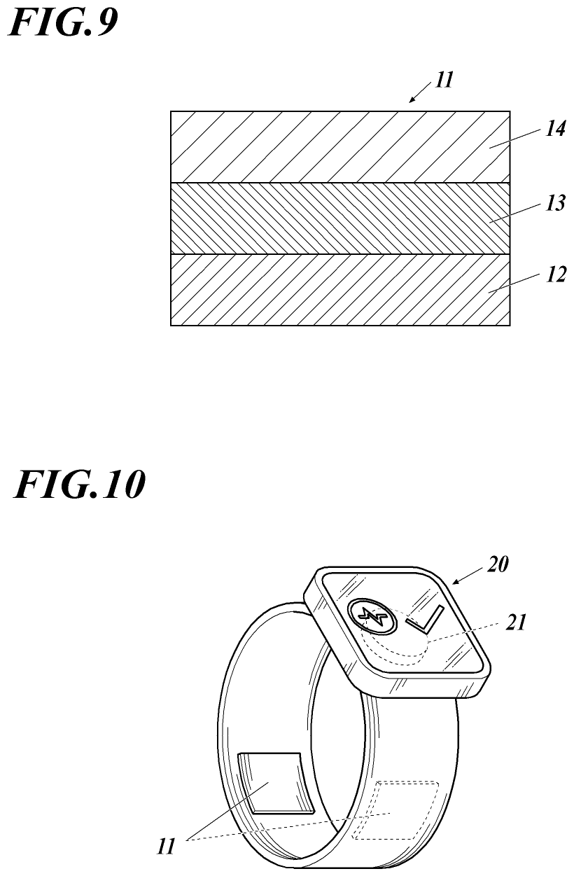

In the production of the light-emitting member 1-2, as shown in FIG. 9, a light-emitting member 1-3 was produced in the same manner except that the cut filter 14 was further provided on the upper part of the wavelength conversion film 13. Further, it was confirmed that the produced light-emitting member 1-3 is a light-emitting member that does not emit the red light of the organic EL element A and emits light in the near-infrared region.

### (Preparation of Pulse Oximeter)

A pulse oximeter was produced by arranging the light-emitting members 1-1 and 1-3 (arranged at the position shown by a symbol 12) produced in Example 4 in such a manner that the light from the light source entering and scattered inside of the wrist was received at a sensor 16 as shown in FIG. 13B.

(Measurement of Oxygen Saturation with Pulse Oximeter)

It was possible to measure an oxygen saturation with the prepared pulse oximeter attached to the wrist.

### Example 8

A transmissive pulse wave sensor at a fingertip using a surface light source was produced and evaluated according to the following method.

A surface light source 12 using the light-emitting member 1-3 produced in Example 7 and a sensor 16 were arranged as shown in FIG. 11B to produce a transmissive pulse wave sensor that enables to perform measurement at a fingertip.

The transmissive pulse wave sensor produced above was attached to an index fingertip 17A, and the pulse wave was measured at a fingertip. As a result, it was possible to obtain a strong pulse wave signal.

### Example 9

A reflective pulse wave sensor at a fingertip using a surface light source was produced and evaluated according to the following method.

A surface light source 12 using the light-emitting member 1-3 produced in Example 7 and a sensor 16 were arranged as shown in FIG. 11C to produce a reflective pulse wave sensor that enables to perform measurement at a fingertip.

FIG. 11C is a schematic view showing reflection type sensing at a fingertip using the surface light source produced above.

The reflective pulse wave sensor produced above was attached to a thumb tip 17A, and the pulse wave was measured at a fingertip. As a result, it was possible to obtain a strong pulse wave signal.

### Example 10

A transmissive pulse wave sensor at a base of a finger using a surface light source was produced and evaluated according to the following method.

A surface light source 12 using the light-emitting member 1-3 produced in Example 7 and a sensor 16 were arranged as shown in FIG. 12B to produce a transmissive pulse wave sensor that enables to perform measurement at a base of a finger.

FIG. 12B is a schematic view showing transmission type sensing at a base of a finger using the surface light source produced above.

The transmissive pulse wave sensor produced above was attached to a base of a thumb 17B, and the pulse wave was measured at a base of a finger. As a result, it was possible to obtain a strong pulse wave signal.

### Comparative Example 1

According to the following method, a transmissive pulse wave sensor at a base of a finger using a point light source, which is a comparative example, was produced and evaluated.

A transmissive pulse wave sensor was prepared by arranging an LED (maximum emission wavelength 850 nm) that emits near-infrared light as a point light source 15 and a sensor 16 as shown in FIG. 12A.

FIG. 12A is a schematic view showing transmission type sensing at a base of a finger using the point light source produced above. It is the same as Example 10 except that a point light source is used.

The transmissive pulse wave sensor produced above was attached to a base of a thumb 17B, and the pulse wave was measured at a base of a finger. As a result, it was not possible to obtain a pulse wave signal.

In comparing the results of Example 10 using the surface light source and Comparative Example 1 using the point light source, a strong pulse wave signal was obtained only in Example 10. It is clear that the cause of this is the difference in the shape of the light source, that is, the use of the surface light source in Example 10 was effective. By newly applying the surface light source according to the present invention to pulse wave measurement, it has become possible to measure pulse waves at a base of a finger, which was difficult to measure with a point light source of the prior art.

### Example 11

A reflective pulse wave sensor on the wrist using a surface light source was produced and evaluated according to the following method.

As shown in FIG. 13C, a surface light source 12 using the light-emitting member 1-3 produced in Example 7 and a sensor 16 were arranged to produce a reflective pulse wave sensor that enables to perform measurement on a wrist.

FIG. 13C is a schematic view showing reflection type sensing on a wrist using the surface light source produced above.

The reflective pulse wave sensor produced above was attached to a wrist 19 and the pulse wave was measured on the wrist. As a result, it was possible to obtain a strong pulse wave signal.

### Comparative Example 2

According to the method described below, a reflective pulse wave sensor on the wrist using a point light source, which is a comparative example, was produced and evaluated.

As shown in FIG. 13D, a reflective pulse wave sensor was produced by arranging an LED (maximum emission wavelength 850 nm) that emits near-infrared light as a point light source 15 and a sensor 12.

FIG. 13D is a schematic view showing transmission type sensing on a wrist using the point light source produced above. It is the same as Example 11 except that a point light source is used.

The reflective pulse wave sensor produced above was attached on a wrist 19 and the pulse wave was measured on the wrist. As a result, almost no pulse wave signal was obtained.

In comparing the results of Example 11 using the surface light source and Comparative Example 2 using the point light source, a strong pulse wave signal was obtained only in Example 11. It is clear that the cause of this is the difference in the shape of the light source, that is, the use of the surface light source in Example 11 was effective. By newly applying the surface light source according to the present invention to pulse wave measurement, it has become possible to measure pulse waves on the wrist, which was difficult to measure with a point light source of the prior art.

### INDUSTRIAL APPLICABILITY

The wavelength conversion film of the present invention can achieve both good quantum yield and emission waveform control. By configuring the wavelength conversion device, it is possible to effectively apply the wavelength conversion device to a biometric authentication device (e.g., venous authentication device), a biometric measurement device (e.g., a pulse wave sensor, a pulse oximeter), and phototherapy.

### DESCRIPTION OF SYMBOLS

1, 13: Wavelength conversion film
2: Excitation light source
3: Detection unit
11: Light-emitting member
12: Surface light source
14: Cut filter
15: Point light source
16: Sensor
17A: Fingertip
17B: Base of finger
18: Bone
19: Wrist
20: Wristband-type electronic device
21: Imaging unit
IR: Near-infrared light
L: Light
P: Intersection
R: Red light emission

1. A wavelength conversion film containing a luminescent dye, wherein, in an absorption spectrum and an emission spectrum, when the spectrums are normalized so that two maximum intensities of an absorption band having a maximum absorption wavelength of a maximum intensity and an emission band having a maximum emission wavelength of a maximum intensity become the same intensity, and when a molar extinction coefficient at an intersection where the normalized absorption band and the normalized emission band overlap in the spectrum is ε, and a content of the luminescent dye included according to a film thickness per 1 cm² of the wavelength conversion film is C (mol), the wavelength conversion film satisfying the following Expression (1),
1.0×10⁻³≤ε×C. Expression (1)

2. The wavelength conversion film described in claim 1, wherein ε×C is 1.5×10⁻³ or more.

3. The wavelength conversion film described in claim 1, wherein when a reflection type emission spectrum detected by a configuration in which an excitation light source and a detection unit are arranged on the same surface side with respect to the wavelength conversion film is defined as an emission spectrum A, and a transmission type emission spectrum detected by a configuration in which the excitation light source and the detection unit are arranged at positions facing each other across the wavelength conversion film is defined as an emission spectrum B, and when peak values of the maximum emission wavelengths of the respective emission spectrums are aligned, a wavelength difference value Δλ at an intensity of 20% on a short-wave side of each of the emission spectrum A and the emission spectrum B is 10 nm or more.

4. The wavelength conversion film described in claim 1, wherein the wavelength difference value Δλ at an intensity of 20% of the short-wave side of the spectrums is 15 nm or more.

5. The wavelength conversion film described in claim 1, wherein an absolute quantum yield is 10% or more.

6. The wavelength conversion film described in claim 1, wherein an absolute quantum yield is 20% or more.

7. The wavelength conversion film described in claim 1, wherein the maximum emission wavelength is 750 nm or more.

8. The wavelength conversion film described in claim 1, wherein the maximum emission wavelength is 800 nm or more.

9. The wavelength conversion film described in claim 1, wherein the wavelength conversion film is formed of the luminescent dye and a transparent matrix resin.

10. The wavelength conversion film described in claim 9, wherein the transparent matrix resin is at least one resin selected from a thermosetting resin, a thermoplastic resin, or a photocurable resin.

11. A wavelength conversion device comprising the wavelength conversion film described in claim 1.

12. A light-emitting member comprising the wavelength conversion film described in claim 1.

13. The light-emitting member described in claim 12, wherein the light source in the light-emitting member is an organic electroluminescent element.

14. An authentication device comprising the light-emitting member described in claim 12.

15. A wristband-type electronic device comprising the authentication device described in claim 14, wherein biometric authentication is performed by imaging a wrist vein.

16. The wristband-type electronic device described in claim 15, wherein the light source is provided on any place of the wristband other than on the same plane as an imaging unit when mounting the device.

17. The biometric device comprising the light-emitting member described in claim 12.

18. The biometric device described in claim 17 being a pulse oximeter that performs measurement at a base of a wrist or a finger.

19. The biometric device described in claim 17 being a pulse wave sensor that performs measurement at the base of a wrist or a finger.
